(19) United States
(12) Patent Application Publication
(10) Pub. No.: US 2025/0235447 A1
(43) Pub. Date: Jul. 24, 2025

(54) METHODS FOR TREATING CARDIOVASCULAR CONDITIONS AND METHODS OF INCREASING THE EFFICIENCY OF CARDIAC METABOLISM

(71) Applicant: IMBRIA PHARMACEUTICALS, INC., Boston, MA (US)

(72) Inventors: Jaikrishna Patel, Cary, NC (US); Paul Chamberlin, Brookline, MA (US)

(21) Appl. No.: 18/703,512

(22) PCT Filed: Oct. 31, 2022
(86) PCT No.: PCT/US2022/048401

§ 371 Date: Apr. 22, 2024

Related U.S. Application Data
(60) Provisional application No. 63/274,389, filed on Nov. 1, 2021.

(51) Int. Cl. A61K 31/496; A61P 9/04

(52) U.S. Cl. CPC A61K 31/496; A61P 9/04

(57) ABSTRACT

The invention provides methods of treating cardiovascular conditions and methods of increasing the efficiency of cardiac metabolism.

### FIELD OF THE INVENTION

The invention relates to methods of treating cardiovascular conditions and methods of increasing the efficiency of cardiac metabolism.

### BACKGROUND

Heart disease is the leading cause of death worldwide, accounting for 15 million deaths across the globe in 2015. In coronary artery disease (CAD), the most common cardiovascular disease, blood flow to the heart muscle is reduced due to accumulation of plaque in the arteries of the heart. Over time, CAD can weaken the heart muscle causing heart failure. Heart failure is a chronic, progressive condition in which the heart is unable to pump enough blood to meet the body's needs. Conditions that can lead to heart failure include diseases of the heart muscle such as hypertrophic cardiomyopathy in which the muscular wall between the two bottom chambers of the heart becomes abnormally thick, thus obstructing blood flow out of the heart. Conditions such as diabetes or pre-diabetes increase the risk of coronary artery disease (CAD), heart failure, and cardiomyopathy.

In heart failure, ischemic heart disease, and diabetic heart disease, decreased cardiac efficiency stems from changes in mitochondrial energy metabolism. Mitochondria are sub-cellular compartments in which metabolites derived from glucose and fatty acids are oxidized to produce high-energy molecules. Increasing fatty acid oxidation in the heart decreases glucose oxidation, and vice versa. Glucose oxidation is a more efficient source of energy, but in certain types of heart disease, such as heart failure, ischemic heart disease, and diabetic cardiomyopathies, fatty acid oxidation predominates in cardiac mitochondria. As a result, the pumping capacity of the heart is reduced.

Existing therapies for treating cardiovascular disease are problematic. Several approaches that focus on restoring blood flow require risky surgical interventions. For example, coronary artery bypass graft is a major surgery associated with various complications. Treatment of obstructive hypertrophic cardiomyopathy includes septal myectomy, ethanol ablation, or an implantable cardioverter defibrillator, all with associated risks of complications.

Many classes of drugs, such as cholesterol-lowering medicine, beta blockers, and calcium channel blockers fail to rectify changes in cardiac energy metabolism. Those existing drugs that redress the balance between glucose oxidation and fatty acid oxidation in cardiac mitochondria have serious shortcomings. Foremost among them is that such drugs address only part of the problem: the reliance on fatty acid oxidation in lieu of glucose oxidation causes a 10% reduction in efficiency in energy production, but patients with heart disease often show a decrease in cardiac efficiency of up to 30%. Consequently, existing approaches to improve cardiac function by altering mitochondrial metabolism are unsatisfactory. Therefore, drugs that do not restore glucose oxidation in the heart have limited efficacy, leaving no safe, effective therapy for millions of people who continue to die from heart disease each year.

### SUMMARY

The invention relates to methods of treating cardiovascular conditions and methods of increasing the efficiency of cardiac metabolism. Particularly, the invention leverages unexpected findings for the use of compositions containing a compound that improves cardiac mitochondrial function to treat cardiac conditions. The compositions contain a compound that is metabolized in the body into multiple products that improve cardiac mitochondrial metabolism by independent but cooperative mechanisms. One set of metabolic products, which may include trimetazidine and its derivatives, shifts cardiac metabolism from fatty acid oxidation to glucose oxidation. Metabolic products in another set serve as precursors for synthesis of nicotinamide adenine dinucleotide (NAD⁺) and thus facilitate mitochondrial respiration. The compositions may be delivered orally, obviating the need for specialized equipment or personnel. The methods are useful for treating a wide variety of cardiovascular conditions as described herein.

The methods of the invention involve use of a compound represented by formula (VII) or (VIII):

A-C(VII) or A-L-C(VIII),

in which A is a compound that shifts cardiac metabolism from fatty acid oxidation to glucose oxidation, L is a linker, and C is a NAD⁺ precursor molecule. A may be covalently linked to C or to L, and L may be covalently linked to C.

The compound that shifts cardiac metabolism from fatty acid oxidation to glucose oxidation may be trimetazidine, etomoxir, oxfenicine, perhexiline, a PPAR agonist, a malonyl CoA decarboxylase inhibitor, or dichloroacetate.

The NAD⁺ precursor molecule may be nicotinic acid, nicotinamide, nicotinamide mononucleotide (NMN), or nicotinamide riboside.

The compound of formula (VII) or (VIII) may be PEGylated with an ethylene glycol moiety. The ethylene glycol moiety may be attached to one or more of A, L, and C. L may be or include an ethylene glycol moiety. The compound may have multiple ethylene glycol moieties, such as one, two three, four, five, or more ethylene glycol moieties. The ethylene glycol moiety may be represented by (CH₂CH₂O)ₓ, in which x=1-15. The ethylene glycol moiety may form a covalent linkage between the compound that shifts cardiac metabolism from fatty acid oxidation to glucose oxidation and the NAD⁺ precursor molecule. The ethylene glycol moiety may be separate from a covalent linkage between the compound that shifts cardiac metabolism from fatty acid oxidation to glucose oxidation and the NAD⁺ precursor molecule. The compound that shifts cardiac metabolism from fatty acid oxidation to glucose oxidation may be a PEGylated form of trimetazidine.

The compound of formula (VII) or the compound of formula (VIII) may include nicotinic acid that is covalently linked to a PEGylated form of trimetazidine. The nicotinic acid may be covalently linked via the PEGylated moiety, i.e., via an ethylene glycol linkage. The nicotinic acid may be covalently linked via the trimetazidine moiety.

The compound of formula (VII) or the compound of formula (VIII) may have a structure represented by formula (X):

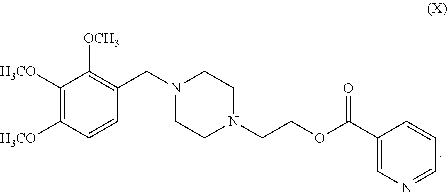 [SMILES: COc1ccc(CN2CCN(CCOC(=O)c3cccnc3)CC2)c(OC)c1OC]

The compounds and compositions may be provided in a dosage form and the dose may be provided by any suitable route or mode of administration. The dose may be provided orally, intravenously, enterally, parenterally, dermally, buccally, topically, transdermally, by injection, subcutaneously, nasally, pulmonarily, or with or on an implantable medical device (e.g., stent or drug-eluting stent or balloon equivalents).

The composition may be provided in one dose per day. The composition may be provided in multiple doses per day. The composition may be provided in two, three, four, five, six, eight, or more doses per day.

The dose may contain from about 10 mg to about 2000 mg, from about 10 mg to about 1000 mg, from about 10 mg to about 800 mg, from about 10 mg to about 600 mg, from about 10 mg to about 400 mg, from about 10 mg to about 300 mg, from about 10 mg to about 200 mg, from about 25 mg to about 2000 mg, from about 25 mg to about 1000 mg, from about 25 mg to about 800 mg, from about 25 mg to about 600 mg, from about 25 mg to about 400 mg, from about 25 mg to about 300 mg, about 25 mg to about 200 mg, from about 50 mg to about 2000 mg, from about 50 mg to about 1000 mg, from about 50 mg to about 800 mg, from about 50 mg to about 600 mg, from about 50 mg to about 400 mg, from about 50 mg to about 300 mg, about 50 mg to about 200 mg, from about 100 mg to about 2000 mg, from about 100 mg to about 1000 mg, from about 100 mg to about 800 mg, from about 100 mg to about 600 mg, from about 100 mg to about 400 mg, from about 100 mg to about 300 mg, about 100 mg to about 200 mg, from about 200 mg to about 2000 mg, from about 200 mg to about 1000 mg, from about 200 mg to about 800 mg, from about 200 mg to about 600 mg, from about 200 mg to about 400 mg, from about 200 mg to about 300 mg, from about 300 mg to about 2000 mg, from about 300 mg to about 1000 mg, from about 300 mg to about 800 mg, from about 300 mg to about 600 mg, or from about 300 mg to about 400 mg of the compound. The dose may contain about 10 mg, about 25 mg, about 50 mg, about 100 mg, about 200 mg, about 300 mg, or about 400 mg of the compound.

The dose or doses may be provided for a defined period. One or more doses may be provided daily for at least one week, at least two weeks, at least three weeks, at least four weeks, at least six weeks, at least eight weeks, at least ten weeks, at least twelve weeks or more.

In certain aspects, the invention provides a method of treating cardiac steatosis or a disorder associated with cardiac steatosis in a subject. The method includes providing to a subject having, or at risk of developing, cardiac steatosis or a disorder associated with cardiac steatosis a composition of a compound having a structure represented by formula (X). In the method, the composition may be provided orally. The composition may be provided in at least one dose per day or may be provided in multiple doses per day at a suitable interval. As an example, the composition may be provided in at least one dose daily for at least two weeks. The dose of the compound of formula (X) may be from about 25 mg to about 1000 mg, from about 50 mg to about 600 mg, and from about 100 mg to about 400 mg. Preferably, the dose may be about 200 mg. The composition may be a modified-release formulation.

In another aspect, the invention provides a method of reducing myocardial triglycerides in a subject by providing to a subject having, or at risk of developing, myocardial disease a composition of a compound having a structure represented by formula (X). In the method, the composition may be provided orally. The composition may be provided in at least one dose per day or may be provided in multiple doses per day at a suitable interval. As an example, the composition may be provided in at least one dose daily for at least two weeks. The dose of the compound of formula (X) may be from about 25 mg to about 1000 mg, from about 50 mg to about 600 mg, and from about 100 mg to about 400 mg. Preferably, the dose may be about 200 mg. The composition may be a modified-release formulation.

The cardiovascular condition may include acute coronary syndrome; aneurysm; angina; atherosclerosis; cardiac adiposity or steatosis including conditions such as aortic stenosis, HIV/ART-associated myocardial steatosis, hypertensive heart disease, pulmonary arterial hypertension, coronary microvascular dysfunction and generalized lipodystrophy; cardiac ischemia-reperfusion injury; cardiomyopathy (inherited or acquired, including obstructive hypertrophic, non-obstructive hypertrophic, dilated, and restrictive forms); cardioprotection (including during cardiac surgery with cardiopulmonary bypass); cerebral vascular disease; chronic coronary syndromes; congenital heart disease; coronary artery disease; coronary heart disease; coronary microvascular dysfunction; diabetic cardiomyopathy (including asymptomatic pre-overt heart failure); heart attack; heart disease; heart failure (all stages and with reduced, mildly reduced or preserved ejection fraction); heart failure after cardiac transplantation in diabetics; hypertension; hypertensive heart disease; ischemic heart disease; ischemia with no obstructive coronary artery disease; lipotoxic cardiomyopathy; metabolic syndrome; microvascular angina; mitochondrial cardiomyopathies; myocardial infarction, obesity cardiomyopathy; pericardial disease; pericardial (or epicardial) fat accumulation; peripheral arterial disease; pulmonary arterial hypertension, right ventricular failure; rheumatic heart disease; stroke; transient ischemic attacks; valvular heart disease (including as medical therapy pre- and/or post-valve repair or replacement); and vasospastic angina.

Aortic stenosis is discussed in Mahmod M, Bull S, Suttie J J, Pal N, Holloway C, Dass S, Myerson S G, Schneider J E, De Silva R, Petrou M, Sayeed R, Westaby S, Clelland C, Francis J M, Ashrafian H, Karamitsos T D, Neubauer S. Myocardial steatosis and left ventricular contractile dysfunction in patients with severe aortic stenosis. Circ Cardiovasc Imaging. 2013 September; 6 (5): 808-16. doi: 10.1161/circimaging.113.000559. Epub 2013 Jul. 5. PMID: 23833283, the entirety of the contents of which are incorporated by reference herein.

HIV/ART-associated myocardial steatosis is discussed in Neilan T G, Nguyen K L, Zaha V G, Chew K W, Morrison L, Ntusi NAB, Toribio M, Awadalla M, Drobni Z D, Nelson M D, Burdo T H, Van Schalkwyk M, Sax P E, Skiest D J, Tashima K, Landovitz R J, Daar E, Wurcel A G, Robbins G K, Bolan R K, Fitch K V, Currier J S, Bloomfield G S, Desvigne-Nickens P, Douglas P S, Hoffmann U, Grinspoon S K, Ribaudo H, Dawson R, Goetz M B, Jain M K, Warner A, Szczepaniak L S, Zanni M V. Myocardial Steatosis Among Antiretroviral Therapy-Treated People With Human Immunodeficiency Virus Participating in the REPRIEVE Trial. J Infect Dis. 2020 Jul. 9; 222 (Suppl 1): S63-S69. doi: 10.1093/infdis/jiaa245. PMID: 32645158; PMCID: PMC7347082, and Holloway C J, Ntusi N, Suttie J, Mahmod M, Wainwright E, Clutton G, Hancock G, Beak P, Tajar A, Piechnik S K, Schneider J E, Angus B, Clarke K, Dorrell L, Neubauer S. Comprehensive cardiac magnetic resonance imaging and spectroscopy reveal a high burden of myocardial disease in HIV patients. Circulation. 2013 Aug. 20; 128 (8): 814-22. doi: 10.1161/circulationaha.113.001719. Epub 2013 Jul. 1. PMID: 23817574 the entirety of the contents of which are incorporated by reference herein.

Hypertensive heart disease is discussed in Sai E, Shimada K, Yokoyama T, Hiki M, Sato S, Hamasaki N, Maruyama M, Morimoto R, Miyazaki T, Fujimoto S, Tamura Y, Aoki S, Watada H, Kawamori R, Daida H. Myocardial triglyceride content in patients with left ventricular hypertrophy: comparison between hypertensive heart disease and hypertrophic cardiomyopathy. Heart Vessels. 2017 February; 32 (2): 166-174. doi: 10.1007/s00380-016-0844-8. Epub 2016 May 3. PMID: 27142065, the entirety of the contents of which are incorporated by reference herein.

Pulmonary arterial hypertension is discussed in Brittain E L, Talati M, Fessel J P, Zhu H, Penner N, Calcutt M W, West J D, Funke M, Lewis G D, Gerszten R E, Hamid R, Pugh M E, Austin E D, Newman J H, Hemnes A R. Fatty Acid Metabolic Defects and Right Ventricular Lipotoxicity in Human Pulmonary Arterial Hypertension. Circulation. 2016 May 17; 133 (20): 1936-44. doi: 10.1161/circulationaha.115.019351. Epub 2016 Mar. 22. PMID: 27006481; PMCID: PMC4870107, the entirety of the contents of which are incorporated by reference herein.

Coronary microvascular dysfunction is discussed in Wei J, Nelson M D, Szczepaniak E W, Smith L, Mehta P K, Thomson L E, Berman D S, Li D, Bairey Merz C N, Szczepaniak L S. Myocardial steatosis as a possible mechanistic link between diastolic dysfunction and coronary microvascular dysfunction in women. Am J Physiol Heart Circ Physiol. 2016 Jan. 1; 310 (1):H14-9. doi: 10.1152/ajpheart.00612.2015. Epub 2015 Oct. 30. PMID: 26519031; PMCID: PMC4865076, the entirety of the contents of which are incorporated by reference herein.

Generalized lipodystrophy is discussed in Nelson M D, Victor R G, Szczepaniak E W, Simha V, Garg A, Szczepaniak L S. Cardiac steatosis and left ventricular hypertrophy in patients with generalized lipodystrophy as determined by magnetic resonance spectroscopy and imaging. Am J Cardiol. 2013 Oct. 1; 112 (7): 1019-24. doi: 10.1016/j.amjcard.2013.05.036. Epub 2013 Jun. 22. PMID: 23800548; PMCID: PMC3779507, the entirety of the contents of which are incorporated by reference herein.

Heart failure after cardiac transplantation in diabetics is discussed in Marfella R, Amarelli C, Cacciatore F, Balestrieri M L, Mansueto G, D'Onofrio N, Esposito S, Mattucci I, Salerno G, De Feo M, D'Amico M, Golino P, Maiello C, Paolisso G, Napoli C. Lipid Accumulation in Hearts Transplanted From Nondiabetic Donors to Diabetic Recipients. J Am Coll Cardiol. 2020 Mar. 24; 75 (11): 1249-1262. doi: 10.1016/j.jacc.2020.01.018. PMID: 32192650, the entirety of the contents of which are incorporated by reference herein.

In another aspect, the invention provides a method of reducing lipotoxicity in a subject, including cardiac lipotoxicity, by providing to a subject having, or at risk of developing, lipotoxicity a composition of a compound having a structure represented by formula (X). In the method, the composition may be provided orally. The composition may be provided in at least one dose per day or may be provided in multiple doses per day at a suitable interval. As an example, the composition may be provided in at least one dose daily for at least two weeks. The dose of the compound of formula (X) may be from about 25 mg to about 1000 mg, from about 50 mg to about 600 mg, and from about 100 mg to about 400 mg. Preferably, the dose may be about 200 mg. The composition may be a modified-release formulation.

In another aspect, the invention provides a method of treating diabetic cardiomyopathy in a subject by providing to a subject having, or at risk of developing, diabetic cardiomyopathy a composition of a compound having a structure represented by formula (X). In the method, the composition may be provided orally. The composition may be provided in at least one dose per day or may be provided in multiple doses per day at a suitable interval. As an example, the composition may be provided in at least one dose daily for at least two weeks. The dose of the compound of formula (X) may be from about 25 mg to about 1000 mg, from about 50 mg to about 600 mg, and from about 100 mg to about 400 mg. Preferably, the dose may be about 200 mg. The composition may be a modified-release formulation.

In still another aspect, the invention provides a method of inducing weight loss in a subject by providing to a subject a composition of a compound having a structure represented by formula (X). In the method, the composition may be provided orally. The composition may be provided in at least one dose per day or may be provided in multiple doses per day at a suitable interval. As an example, the composition may be provided in at least one dose daily for at least two weeks. The dose of the compound of formula (X) may be from about 25 mg to about 1000 mg, from about 50 mg to about 600 mg, and from about 100 mg to about 400 mg. Preferably, the dose may be about 200 mg. The composition may be a modified-release formulation.

In another aspect, the invention provides a method of preventing or treating cardiac dysfunction (subclinical or symptomatic) and/or improving cardiac energetics in a subject by providing to a subject having an elevated level of HbA1c (i.e. diabetic or pre-diabetic) at least one dose per day of a composition of a compound having a structure represented by formula (X). The elevated level of HbA1c may be greater than 6.0%, 6.5% or 7%. The dose of the compound of formula (X) may be at least one dose provided orally. As an example, the compound of formula (X) may be provided in at least one dose per day or may be provided in multiple doses per day at a suitable interval. The composition may be provided in at least one dose daily for at least two weeks. The dose of the compound of formula (X) may be from about 25 mg to about 1000 mg, from about 50 mg to about 600 mg, and from about 100 mg to about 400 mg. Preferably, the dose may be about 200 mg. The composition may be a modified-release formulation.

### BRIEF DESCRIPTION OF THE DRAWINGS

FIG. 1 shows a schematic of the study design for testing the safety and efficacy of IMB-1018972.

FIG. 2 is a table of the disposition of subjects of an FIH study of IMB-1018972.

FIG. 3 is a Schedule of Assessments for SAD part Group A5 of an FIH study of IMB-1018972.

FIG. 4 is a table of assessments given for the SAD part (and integrated FE arm) Groups A1 to A4 of an FIH study of IMB-1018972.

FIG. 5 is a table of assessments given for the MAD part of an FIH study of IMB-1018972.

FIG. 6 is a table of analysis data sets for the SAD Part (and integrated FE Arm) per dose level and total for IMB-1018972 of an FIH study of IMB-1018972.

FIG. 7 is a table of analysis data sets for the MAD Part per dose level and total for IMB-1018972 of an FIH study of IMB-1018972.

FIG. 8 is a table of a summary of demographic characteristics—SAD Part (and Integrated F E Arm) (Safety Set of an FIH study of IMB-1018972.

FIG. 9 is a table of a summary of demographic characteristics—MAD Part (Safety Set) of an FIH study of IMB-1018972.

FIG. 10 is a table of the Extent of Exposure—SAD Part (and Integrated FE Arm) (Safety Set) of an FIH study of IMB-1018972.

FIG. 11 is a table of the Extent of Exposure—MAD Part of an FIH study of IMB-1018972.

FIG. 12 is a graph of Geometric Mean IMB-1028814 Plasma Concentration-Time Profiles (Linear)—SAD Part (PK Set) of an FIH study of IMB-1018972.

FIG. 13 is a graph of Geometric Mean IMB-1028814 Plasma Concentration-Time Profiles (Semi-Logarithmic)—SAD Part (PK Set) of an FIH study of IMB-1018972.

FIG. 14 is a graph of Geometric Mean Trimetazidine Plasma Concentration-Time Profiles (Linear)—SAD Part (PK Set) of an FIH study of IMB-1018972.

FIG. 15 is a graph of Geometric Mean Trimetazidine Plasma Concentration-Time Profiles (Semi-Logarithmic)—SAD Part (PK Set) of an FIH study of IMB-1018972.

FIG. 16 is a graph of Geometric Mean IMB-1028814+Trimetazidine Plasma Concentration-Time Profiles (Semi-Logarithmic)—SAD Part (PK Set) of an FIH study of IMB-1018972.

FIG. 17 is a graph of Geometric Mean IMB-1028814+Trimetazidine Plasma Concentration-Time Profiles (Semi-Logarithmic)—SAD Part (PK Set) of an FIH study of IMB-1018972.

FIG. 18 is a table of Summary Statistics (Geometric Mean [Range]) of IMB-1028814, Trimetazidine, and IMB-1028814+Trimetazidine Plasma Pharmacokinetic Parameters—SAD Part (PK Set) of an FIH study of IMB-1018972.

FIG. 19 is a table of Exploratory Analysis of Dose Proportionality for IMB-1028814 and Trimetazidine over the Dose Range of 50 mg to 400 mg IMB-1018972 under Faster Conditions—SAD Part (PK Set) of an FIH study of IMB-1018972.

FIG. 20 is a graph of Plot of Combined Individual and Geometric Mean Dose-Normalized IMB-1028814 Cₘₐₓ over the Dose Range of 50 mg to 400 mg IMB-1018972 under Fasted Conditions—SAD Part (PK Set) of an FIH study of IMB-1018972.

FIG. 21 is a graph of Plot of Combined Individual and Geometric Mean Dose-Normalized IMB-1028814 AUC₀₋ₜ over the Dose Range of 50 mg to 400 mg IMB-1018972 under Fasted Conditions—SAD Part (PK Set) of an FIH study of IMB-1018972.

FIG. 22 is a graph of Plot of Combined Individual and Geometric Mean Dose-Normalized IMB-1028814 AUC_{0-inf} over the Dose Range of 50 mg to 400 mg IMB-1018972 under Fasted Conditions—SAD Part (PK Set) of an FIH study of IMB-1018972.

FIG. 23 is a graph of Plot of Combined Individual and Geometric Mean Dose-Normalized Trimetazidine Cₘₐₓ over the Dose Range of 50 mg to 400 mg IMB-1018972 under Fasted Conditions—SAD Part (PK Set) of an FIH study of IMB-1018972.

FIG. 24 is a graph of Plot of Combined Individual and Geometric Mean Dose-Normalized Trimetazidine AUC₀₋ₜ over the Dose Range of 50 mg to 400 mg IMB-1018972 under Fasted Conditions—SAD Part (PK Set) of an FIH study of IMB-1018972.

FIG. 25 is a graph of Plot of Combined Individual and Geometric Mean Dose-Normalized Trimetazidine AUC_{0-inf} over the Dose Range of 50 mg to 400 mg IMB-1018972 under Fasted Conditions—SAD Part (PK Set) of an FIH study of IMB-1018972.

FIG. 26 is a graph of Geometric Mean IMB-1028814 Plasma Concentration-Time Profiles (Linear)—FE Arm of SAD Part (PK Set) of an FIH study of IMB-1018972.

FIG. 27 is a graph of Geometric Mean IMB-1028814 Plasma Concentration-Time Profiles (Semi-Logarithmic Scale)—FE Arm of SAD Part (PK Set) of an FIH study of IMB-1018972.

FIG. 28 is a graph of Geometric Mean Trimetazidine Plasma Concentration-Time Profiles (Linear)—FE Arm of SAD Part (PK Set) of an FIH study of IMB-1018972.

FIG. 29 is a graph of Geometric Mean Trimetazidine Plasma Concentration-Time Profiles (Semi-Logarithmic Scale)—FE Arm of SAD Part (PK Set) of an FIH study of IMB-1018972.

FIG. 30 is a graph of Geometric Mean IMB-1028814+Trimetazidine Plasma Concentration-Time Profiles (Linear)—FE Arm of SAD Part (PK Set) of an FIH study of IMB-1018972.

FIG. 31 is a graph of Geometric Mean IMB-1028814+Trimetazidine Plasma Concentration-Time Profiles (Semi-Logarithmic Scale)—FE Arm of SAD Part (PK Set) of an FIH study of IMB-1018972.

FIG. 32 is a table of Summary Statistics (Geometric Mean [Range]) of IMB-1028814, Trimetazidine, and IMB-128814+Trimetazidine, and IMB-1028814+Trimetazidine Plasma Pharmacokinetic Parameters-FE Arm of SAD Part (PK Set) of an FIH study of IMB-1018972.

FIG. 33 is a table of Exploratory Analysis of Food Effect for IMB-1028814 and Trimetazidine following Administration of 150 mg IMB-1018972—FE Arm of SAD Part (PK Set) of an FIH study of IMB-1018972.

FIG. 34 is a table of Summary Statistics (Arithmetic Mean [SD]) of Urine Pharmacokinetic Parameters for IMB-1028814, Trimetazidine, and IMB-1028814+Trimetazidine—SAD Part (PK Set) of an FIH study of IMB-1018972.

FIG. 35 is a graph of Geometric Mean IMB-1028814 Plasma Concentration-Time Profiles from Day 1 through Day 14 (Linear)—MAD Part (PK Set) of an FIH study of IMB-1018972.

FIG. 36 is a graph of Geometric Mean IMB-1028814 Plasma Concentration-Time Profiles from Day 1 through Day 14 (Semi-Logarithmic Scale)—MAD Part (PK Set) of an FIH study of IMB-1018972.

FIG. 37 is a graph of Geometric Mean Trimetazidine Plasma Concentration-Time Profiles from Day 1 through Day 14 (Linear)—MAD Part (PK Set) of an FIH study of IMB-1018972.

FIG. 38 is a graph of Geometric Mean Trimetazidine Plasma Concentration-Time Profiles from Day 1 through Day 14 (Semi-Logarithmic Scale)—MAD Part (PK Set) of an FIH study of IMB-1018972.

FIG. 39 is a graph of Geometric Mean IMB-1028814+Trimetazidine Plasma Concentration-Time Profiles from Day 1 through Day 14 (Linear)—MAD Part (PK Set) of an FIH study of IMB-1018972.

FIG. 40 is a graph of Geometric Mean IMB-1028814+Trimetazidine Plasma Concentration-Time Profiles from Day 1 through Day 14 (Semi-Logarithmic Scale)—MAD Part (PK Set) of an FIH study of IMB-1018972.

FIG. 41 is a graph of Geometric Mean IMB-1028814 Plasma Concentration-Time Profiles from Day 1 through Day 14 (Linear)—MAD Part (PK Set) of an FIH study of IMB-1018972.

FIG. 42 is a graph of Geometric Mean IMB-1028814 Plasma Concentration-Time Profiles from Day 1 through Day 14 (Semi-Logarithmic Scale)—MAD Part (PK Set) of an FIH study of IMB-1018972.

FIG. 43 is a graph of Geometric Mean Trimetazidine Plasma Concentration-Time Profiles after Dosing on Day 1 through Day 14 (Linear)—MAD Part (PK Set) of an FIH study of IMB-1018972.

FIG. 44 is a graph of Geometric Mean Trimetazidine Plasma Concentration-Time Profiles after Dosing on Day 1 through Day 14 (Semi-Logarithmic Scale)—MAD Part (PK Set) of an FIH study of IMB-1018972.

FIG. 45 is a graph of Geometric Mean IMB-1028814+Trimetazidine Plasma Concentration-Time Profiles after Dosing on Day 1 through Day 14 (Linear)—MAD Part (PK Set) of an FIH study of IMB-1018972.

FIG. 46 is a graph of Geometric Mean IMB-1028814+Trimetazidine Plasma Concentration-Time Profiles after Dosing on Day 1 through Day 14 (Semi-Logarithmic Scale)—MAD Part (PK Set) of an FIH study of IMB-1018972.

FIG. 47 is a table of Summary Statistics (Geometric Mean [Range]) of IMB-1028814, Trimetazidine, and IMB-1028814+Trimetazidine Plasma Pharmacokinetic Parameters—MAD Part (PK Set) of an FIH study of IMB-1018972.

FIG. 48A and FIG. 48B is a table Summary of All TEAEs by System Organ Class, Preferred Term and Treatment—SAD Part (and integrated FE Arm) (Safety Set) with the following notifications: of an FIH study of IMB-1018972.

FIG. 49A and FIG. 49B is a table Summary of All TEAEs by System Organ Class, Preferred Term and Treatment—MAD Part (Safety Set) of an FIH study of IMB-1018972.

FIG. 50 is a table Summary of All TEAEs by Treatment, Relationship, and Severity—SAD Part (and Integrated FE Arm) (Safety Set) of an FIH study of IMB-1018972.

FIG. 51 is a table Summary of All TEAEs by Treatment, Relationship, and Severity—MAD Part (Safety Set) of an FIH study of IMB-1018972.

FIG. 52 is baseline characteristics of randomized participants for Example 3 pharmacodynamic study to evaluate the impact of formula (X) on myocardial energetics and metabolism in Type 2 diabetes.

FIG. 53 is baseline characteristics of completers for Example 3 pharmacodynamic study to evaluate the impact of formula (X) on myocardial energetics and metabolism in Type 2 diabetes, including baseline cardiac imaging findings in those treated for 4 weeks.

FIG. 54 is adverse effects as of the data cut-off date of Sep. 20, 2021 for Example 3 pharmacodynamic study to evaluate the impact of formula (X) on myocardial energetics and metabolism in Type 2 diabetes.

FIG. 55 are graphs of resting myocardial PCr/ATP combined, 4- and 8-week cohorts for Example 3 pharmacodynamic study to evaluate the impact of formula (X) on myocardial energetics and metabolism in Type 2 diabetes.

FIG. 56 are graphs of myocardial triglyceride (MTG) combined 4- and 8-week cohorts for Example 3 pharmacodynamic study to evaluate the impact of formula (X) on myocardial energetics and metabolism in Type 2 diabetes.

FIG. 57 are graphs of body weight combined 4- and 8-week cohorts for Example 3 pharmacodynamic study to evaluate the impact of formula (X) on myocardial energetics and metabolism in Type 2 diabetes.

FIG. 58 is correlation analysis data with plots of change in PCr/ATP and baseline HbA1c and baseline fasting glucose for Example 3 pharmacodynamic study to evaluate the impact of formula (X) on myocardial energetics and metabolism in Type 2 diabetes.

FIG. 59 is correlation analysis data with plots of change in PCr/ATP and change in myocardial triglycerides and baseline myocardial triglycerides for Example 3 pharmacodynamic study to evaluate the impact of formula (X) on myocardial energetics and metabolism in Type 2 diabetes.

FIG. 60 is correlation analysis data with plots of change in body weight and baseline HbA1c and change in PCr/ATP for Example 3 pharmacodynamic study to evaluate the impact of formula (X) on myocardial energetics and metabolism in Type 2 diabetes.

FIG. 61 is correlation analysis data with plots of change in myocardial triglycerides and baseline myocardial triglycerides and baseline HbA1c for Example 3 pharmacodynamic study to evaluate the impact of formula (X) on myocardial energetics and metabolism in Type 2 diabetes.

FIG. 62 is correlation analysis data with plots of change in body weight and change in myocardial triglycerides for Example 3 pharmacodynamic study to evaluate the impact of formula (X) on myocardial energetics and metabolism in Type 2 diabetes.

FIG. 63 is correlation analysis data and plots for change in PCr/ATP with change in myocardial triglycerides, absolute and % change for Example 3 pharmacodynamic study to evaluate the impact of formula (X) on myocardial energetics and metabolism in Type 2 diabetes.

FIG. 64 is correlation analysis with plots of change in PCr/ATP with change in myocardial triglycerides, absolute and percent change following removal of an outlier for Example 3 pharmacodynamic study to evaluate the impact of formula (X) on myocardial energetics and metabolism in Type 2 diabetes.

FIG. 65 is correlation analysis data with plots of baseline myocardial triglycerides with HbA1c (%) and other pertinent plots for comparison for Example 3 pharmacodynamic study to evaluate the impact of formula (X) on myocardial energetics and metabolism in Type 2 diabetes.

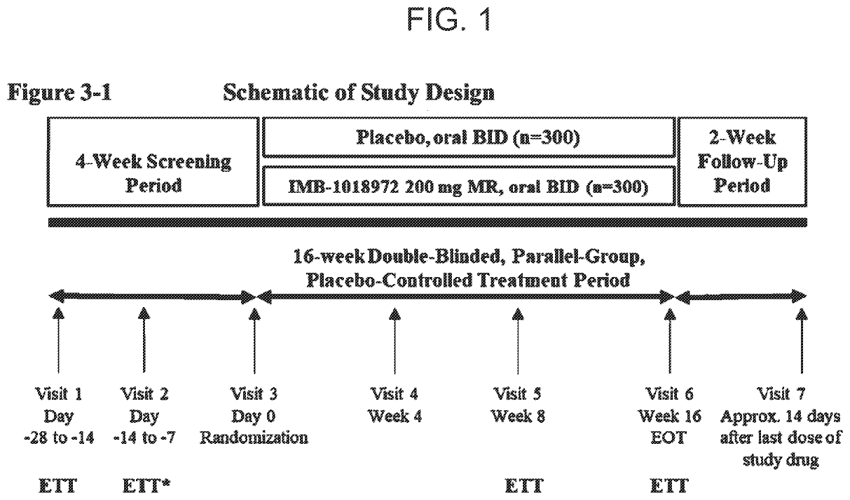

### DETAILED DESCRIPTION

The invention provides methods for administering compositions containing a compound that improves cardiac mitochondrial function to treat cardiac conditions. In some embodiments, the methods include providing such a composition to subject one or more times per day. Because the compositions may be formulated for oral administration, the methods are simple and may be performed by a patient without direct medical supervision. The methods may be used to treat cardiovascular conditions as described herein.

In many types of heart disease, the overall efficiency of energy production by cardiac mitochondria is diminished. In part, this is due to an increased reliance on fatty acid oxidation over glucose oxidation in many types of heart disease. Archetypal examples of this include diabetic cardiomyopathy and obesity cardiomyopathy. Glucose oxidation is a more efficient pathway for energy production, as measured by the number of ATP molecules produced per 02 molecule consumed (P/O˜2.58), than is fatty acid oxidation (P/O˜2.3) and that of the ketone body β-hydroxybutyrate (P/O˜2.5). The importance of this is highlighted by the observation that the heart utilizes more oxygen/gram of tissue than any other organ. Glucose oxidation also consumes less NAD⁺ than oxidation of a long-chain fatty acid, palmitate (10 and 31, respectively), hence a shift towards glucose oxidation is expected to increase the cardiomyocyte NAD⁺ pool and NAD⁺/NADH ratio, and this can be further potentiated by concomitant use of an NAD⁺ precursor such as nicotinic acid. Cardiac NAD⁺ and the NAD⁺/NADH ratio are reduced in pathologies such as heart failure, cardiac pressure overload and in diabetic cardiomyopathy. However, other metabolic changes contribute to decreased cardiac efficiency in patients with heart disease. For example, overall mitochondrial oxidative metabolism can be impaired in heart failure, and energy production is decreased in ischemic heart disease due to a limited supply of oxygen at rest or under conditions of increased myocardial oxygen demand, such as exercise inducing ischemia. As a corollary, stimulation of myocardial glucose oxidation will improve post-ischemic recovery and cardiac efficiency following a period of ischemia and reperfusion. In addition to the reduction in mitochondrial oxidative capacity, the failing heart is characterized by increased glycolysis uncoupled from glucose oxidation, reducing energy production (2 compared with 31 ATP molecules per glucose molecule if the pyruvate from glycolysis is oxidized) and generating lactate leading to intracellular H⁺ accumulation impairing cellular function and intracellular Ca²⁺ homeostasis.

Increased reliance of the myocardium on fatty acids decreases cardiac efficiency via other mechanisms including activation of mitochondrial uncoupling proteins (uncoupling ATP generation from oxidative metabolism) and through futile cycling of fatty acid intermediates resulting in ATP consumption for non-contractile purposes.

Given the hearts unrelenting high energy requirements to meet the demands of contractility (the heart would exhaust its ATP content in ˜2-10 seconds if not replaced), reduced efficiency of energy generation by the heart and the ensuing energy deficit has profound adverse consequences. As a corollary, energetic impairment (which can be measured non-invasively by the phosphocreatine/ATP ratio) is a major feature and contributor to most forms of heart disease.

Glucose oxidation and fatty acid oxidation are energy-producing metabolic pathways that compete with each other for substrates. In glucose oxidation, glucose is broken down to pyruvate via glycolysis in the cytosol of the cell. Pyruvate then enters the mitochondria, where it is converted to acetyl coenzyme A (acetyl-CoA). In beta-oxidation of fatty acids, which occurs in the mitochondria, two-carbon units from long-chain fatty acids are sequentially converted to acetyl-CoA.

The remaining steps in energy production from glucose oxidation of glucose and fatty acid oxidation are common to the two pathways. Acetyl-CoA is oxidized to carbon dioxide (CO₂) via the citric acid cycle, which results in the conversion of nicotinamide adenine dinucleotide (NAD⁺) to its reduced form, NADH. NADH, in turn, drives the mitochondrial electron transport chain. The electron transport chain comprises a series of four mitochondrial membrane-bound complexes that transfer electrons via redox reactions. In doing so, the complexes pump protons across the membrane to create a proton gradient. The redox reactions of the electron transport chain require molecular oxygen (O₂). In the final step of mitochondrial energy production, the proton gradient enables another membrane-bound enzymatic complex to form high-energy ATP molecules, which are the source of energy for most cellular reactions.

The methods of the invention improve cardiac efficiency by using multiple mechanisms to alter mitochondrial metabolism. In certain embodiments, the methods entail providing compounds that are metabolized in the body into multiple products that have different effects. A first metabolic product or set of metabolic products shifts cardiac metabolism from fatty acid oxidation to glucose oxidation, and a second product or set of products modulates the NAD⁺/NADH redox couple and promotes mitochondrial respiration. Thus, administering such compounds triggers a change in the pathway used to produce energy and concomitantly improves overall mitochondrial oxidative function. Consequently, the methods of the invention are more effective at restoring cardiac capacity in patients with heart disease than are other methods that target a single metabolic deficiency. Moreover, such methods avoid the use of risky surgical procedures that can lead to serious complications.

### Dosing Methods

In certain embodiments, methods of the invention include providing a composition containing a compound that improves cardiac mitochondrial function to a subject.

The dose may be provided by any suitable route or mode of administration. The dose may be provided orally, intravenously, enterally, parenterally, dermally, buccally, topically, transdermally, by injection, subcutaneously, nasally, pulmonarily, or with or on an implantable medical device (e.g., stent or drug-eluting stent or balloon equivalents).

Doses may be provided at any suitable interval. For example and without limitation, doses may be provided once per day, twice per day, three times per day, four times per day, five times per day, six times per day, eight times per day, once every 48 hours, once every 36 hours, once every 24 hours, once every 12 hours, once every 8 hours, once every 6 hours, once every 4 hours, once every 3 hours, once every two days, once every three days, once every four days, once every five days, once every week, twice per week, three times per week, four times per week, or five times per week.

The dose may contain a defined amount of the compound that improves cardiac mitochondrial function. For example and without limitation, the dose may contain from about 10 mg to about 2000 mg, from about 10 mg to about 1000 mg, from about 10 mg to about 800 mg, from about 10 mg to about 600 mg, from about 10 mg to about 400 mg, from about 10 mg to about 300 mg, from about 10 mg to about 200 mg, from about 25 mg to about 2000 mg, from about 25 mg to about 1000 mg, from about 25 mg to about 800 mg, from about 25 mg to about 600 mg, from about 25 mg to about 400 mg, from about 25 mg to about 300 mg, about 25 mg to about 200 mg, from about 50 mg to about 2000 mg, from about 50 mg to about 1000 mg, from about 50 mg to about 800 mg, from about 50 mg to about 600 mg, from about 50 mg to about 400 mg, from about 50 mg to about 300 mg, about 50 mg to about 200 mg, from about 100 mg to about 2000 mg, from about 100 mg to about 1000 mg, from about 100 mg to about 800 mg, from about 100 mg to about 600 mg, from about 100 mg to about 400 mg, from about 100 mg to about 300 mg, about 100 mg to about 200 mg, from about 200 mg to about 2000 mg, from about 200 mg to about 1000 mg, from about 200 mg to about 800 mg, from about 200 mg to about 600 mg, from about 200 mg to about 400 mg, from about 200 mg to about 300 mg, from about 300 mg to about 2000 mg, from about 300 mg to about 1000 mg, from about 300 mg to about 800 mg, from about 300 mg to about 600 mg, or from about 300 mg to about 400 mg of the compound. The dose may contain about 10 mg, about 25 mg, about 50 mg, about 100 mg, about 200 mg, about 300 mg, or about 400 mg of the compound.

The dose may be provided in a single dosage, i.e., the dose may be provided as a single tablet, capsule, pill, etc. Alternatively, the dose may be provided in a divided dosage, i.e., the dose may be provided as multiple tablets, capsules, pills, etc.

The dosing may continue for a defined period. For example and without limitation, doses may be provided for at least one week, at least two weeks, at least three weeks, at least four weeks, at least six weeks, at least eight weeks, at least ten weeks, at least twelve weeks or more.

The subject may be a human. The subject may be a human that has a cardiovascular condition, such as one of those described below. The subject may be a human that is at risk of developing a cardiovascular condition, such as one of those described above. A subject may be at risk of developing a condition if the subject does not meet established criteria for diagnosis of the condition but has one or more symptoms, markers, or other factors that indicate the subject is likely to meet the diagnostic criteria for the condition in the future. The subject may be a pediatric, a newborn, a neonate, an infant, a child, an adolescent, a pre-teen, a teenager, an adult, or an elderly subject. The subject may be in critical care, intensive care, neonatal intensive care, pediatric intensive care, coronary care, cardiothoracic care, surgical intensive care, medical intensive care, long-term intensive care, an operating room, an ambulance, a field hospital, or an out-of-hospital field setting such as an outpatient or community setting.

### Compounds

Certain embodiments of the invention include providing to a subject a composition containing a compound represented by formula (VII) or (VIII):

A-C(VII) or A-L-C(VIII),

in which A is a compound that shifts cardiac metabolism from fatty acid oxidation to glucose oxidation, L is a linker, and C is a NAD⁺ precursor. Examples of each component are described in detail below. A may be covalently linked to C or to L, and L may be covalently linked to C.

The compound of formula (VII) may include nicotinic acid that is covalently linked to a PEGylated form of trimetazidine. The nicotinic acid may be covalently linked via a PEGylated moiety, i.e., via an ethylene glycol linkage. The nicotinic acid may be covalently linked via the trimetazidine moiety.

The compound of formula (VII) or the compound of formula (VIII) may have a structure represented by formula (X):

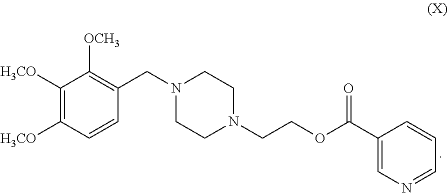 [SMILES: COc1ccc(CN2CCN(CCOC(=O)c3cccnc3)CC2)c(OC)c1OC]

Compounds of formulas (VII), (VIII), and (X) are described in, for example, International Patent Publication No. WO 2018/236745, the contents of which are incorporated herein by reference.

Compounds that Shift Cardiac Metabolism from Fatty Acid Oxidation to Glucose Oxidation

Component A may be any suitable compound that shifts cardiac metabolism from fatty acid oxidation to glucose oxidation. Such compounds can be classified based on their mechanism of action. See Fillmore, N., et al., Mitochondrial fatty acid oxidation alterations in heart failure, ischemic heart disease and diabetic cardiomyopathy, Brit. J. Pharmacol. 171:2080-2090 (2014), the contents of which are incorporated herein by reference.

One class of glucose-shifting compounds includes compounds that inhibit fatty acid oxidation directly. Compounds in this class include inhibitors of malonyl CoA decarboxylase (MCD), carnitine palmitoyl transferase 1 (CPT-1), or mitochondrial fatty acid oxidation. Mitochondrial fatty acid oxidation inhibitors include trimetazidine and other compounds described in International Patent Publication No. WO 2002/064576, the contents of which are incorporated herein by reference. Trimetazidine binds to distinct sites on the inner and outer mitochondrial membranes and affects both ion permeability and metabolic function of mitochondria. Morin, D., et al., Evidence for the existence of [³H]-trimetazidine binding sites involved in the regulation of the mitochondrial permeability transition pore, Brit. J. Pharmacol. 123:1385-1394 (1998), the contents of which are incorporated herein by reference. MCD inhibitors include CBM-301106, CBM-300864, CBM-301940, 5-(1,1,1,3,3,3-hexafluoro-2-hydroxypropan-2-yl)-4,5-dihydroisoxazole-3-carboxamides, methyl 5-(N-(4-(1,1,1,3,3,3-hexafluoro-2-hydroxypropan-2-yl)phenyl) morpholine-4-carboxamido) pentanoate, and other compounds described in Chung, J. F., et al., Discovery of Potent and Orally Available Malonyl-CoA Decarboxylase Inhibitors as Cardioprotective Agents, J. Med. Chem. 49:4055-4058 (2006); Cheng J. F. et al., Synthesis and structure-activity relationship of small-molecule malonyl coenzyme A decarboxylase inhibitors, J. Med. Chem. 49:1517-1525 (2006); U.S. Patent Publication No. 2004/0082564; and International Patent Publication No. WO 2002/058698, the contents of each of which are incorporated herein by reference. CPT-1 inhibitors include oxfenicine, perhexiline, etomoxir, and other compounds described in International Patent Publication Nos. WO 2015/018660; WO 2008/109991; WO 2009/015485; and WO 2009/156479; and U.S. Patent Publication No. 2011/0212072, the contents of each of which are incorporated herein by reference.

Another class of glucose-shifting compounds includes compounds that stimulate glucose oxidation directly. Examples of such compounds are described in U.S. Patent Publication No. 2003/0191182; International Patent Publication No. WO 2006/117686; U.S. Pat. No. 8,202,901, the contents of each of which are incorporated herein by reference.

Another class of glucose-shifting compounds includes compounds that decrease the level of circulating fatty acids that supply the heart. Examples of such compounds include agonists of PPARα and PPARγ, including fibrate drugs, such as clofibrate, gemfibrozil, ciprofibrate, bezafibrate, and fenofibrate, and thiazolidinediones, GW-9662, and other compounds described in U.S. Pat. No. 9,096,538, the contents of which are incorporated herein by reference.

### Linkers

Component L may be any suitable linker. Preferably, the linker can be cleaved in vivo to release components A and B. The linker may be an alkoxy group. The linker may be polyethylene glycol of any length. The linker may be represented by (CH₂CH₂O)ₓ, in which x=1-15 or (CH₂CH₂O)ₓ, in which x=1-3. Other suitable linkers include 1,3-propanediol, diazo linkers, phosphoramidite linkers, disulfide linkers, cleavable peptides, iminodiacetic acid linkers, thioether linkers, and other linkers described in Leriche, G., et al., Cleavable linkers in chemical biology, Bioorg. Med. Chem. 20:571-582 (2012); International Patent Publication No. WO 1995000165; and U.S. Pat. No. 8,461,117, the contents of each of which are incorporated herein by reference.

### NAD⁺ Precursor Molecules

Component C may be any molecule that can serve as a precursor to NAD⁺ in vivo. NAD⁺ is an important oxidizing agent that acts as a coenzyme in multiple reactions of the citric acid cycle as well as glycolysis and in the conversion of pyruvate to acetyl-CoA by pyruvate dehydrogenase (PDH). In these reactions, NAD⁺ is reduced to NADH. Conversely, NADH is oxidized back to NAD⁺ when it donates electrons to mitochondrial electron transport chain. In humans, NAD⁺ can be synthesized de novo from tryptophan, but not in quantities sufficient to meet the continual cellular demands for NAD⁺. Consequently, NAD⁺ is also synthesized via a salvage pathway, which uses precursors that must be supplied from the diet. Among the precursors used by the salvage pathway for NAD⁺ synthesis are nicotinic acid (via the Preiss-Handler pathway), nicotinamide, and nicotinamide riboside, the latter two generating nicotinamide mononucleotide (NMN). By providing a NAD⁺ precursor, such as nicotinic acid, nicotinamide, NMN, or nicotinamide riboside, the compound facilitates NAD⁺ synthesis, stabilization and/or expansion of the intracellular NAD⁺ pool and, reflecting the role of NAD⁺ as the main hydride acceptor in intermediary metabolism, support of cellular energy producing metabolic pathways in both cytosol and mitochondria. This approach will also support signaling pathways which utilize NAD+ as a co-substrate, e.g. ADP-ribose transferases and the sirtuins (NAD⁺-dependent protein deacetylases), regulating DNA repair and post-translational protein modifications. NAD⁺ redox imbalance has been implicated in the pathogenesis of a range of cardiovascular, metabolic, senescent and degenerative conditions, including diabetic cardiomyopathy and heart failure.

The inclusion of a NAD⁺ precursor in compounds of the invention allows the compounds to stimulate energy production in cardiac mitochondria in multiple ways. Component A shifts cardiac metabolism from fatty acid oxidation to glucose oxidation, which is inherently more efficient. The NAD⁺ precursor provides an essential coenzyme that cycles between oxidized and reduced forms to promote respiration. In the oxidized form, NAD⁺ drives reactions of the citric acid cycle. In the reduced form, NADH promotes electron transport to create a proton gradient that enables ATP synthesis. Consequently, the chemical potential resulting from oxidation of acetyl CoA is efficiently converted to ATP that can be used for various cellular functions.

The NAD⁺ precursor molecule may be covalently attached to the compound in any suitable manner. For example, it may linked to A or L, and it may be attached directly or via another linker. Preferably, it is attached via a linker that can be cleaved in vivo. The NAD⁺ precursor molecule may be attached via a 1,3-propanediol linkage.

### PEGylation

The compound may be covalently attached to one or more molecules of polyethylene glycol (PEG), i.e., the compound may be PEGylated. In many instances, PEGylation of molecules reduces their immunogenicity, which prevents the molecules from being cleared from the body and allows them to remain in circulation longer. The ethylene glycol moiety may serve as a linker, as described above in relation to Component L, or it may be attached to only one component, e.g., Component A, L, or C, of the compound. The ethylene glycol moiety may be separate from a covalent linkage between the compound that shifts cardiac metabolism from fatty acid oxidation to glucose oxidation and the NAD⁺ precursor molecule.

The compound may contain a PEG polymer of any size. For example, the PEG polymer may have from 1-500 (CH₂CH₂O) units. The ethylene glycol moiety may be represented by (CH₂CH₂O)ₓ, in which x=1-15. The PEG polymer may have any suitable geometry, such as a straight chain, branched chain, star configuration, or comb configuration. The compound may be PEGylated at any site. For example, the compound may be PEGylated on component A, component L (if present), or the NAD⁺ precursor. The compound may be PEGylated at multiple sites. For a compound PEGylated at multiple sites, the various PEG polymers may be of the same or different size and of the same or different configuration.

The compound that shifts cardiac metabolism from fatty acid oxidation to glucose oxidation may be PEGylated with an ethylene glycol moiety. The compound that shifts cardiac metabolism from fatty acid oxidation to glucose oxidation may have multiple ethylene glycol moieties, such as one, two three, four, five, or more ethylene glycol moieties. The ethylene glycol moiety may be represented by (CH₂CH₂O)ₓ, in which x=1-15. The ethylene glycol moiety may form a covalent linkage between the compound that shifts cardiac metabolism from fatty acid oxidation to glucose oxidation and the NAD⁺ precursor molecule. The ethylene glycol moiety may be separate from a covalent linkage between the compound that shifts cardiac metabolism from fatty acid oxidation to glucose oxidation and the NAD⁺ precursor molecule.

The compound of formula (VII) may include nicotinic acid that is covalently linked to a PEGylated form of trimetazidine. The nicotinic acid may be covalently linked via a PEGylated moiety, i.e., via an ethylene glycol linkage. The nicotinic acid may be covalently linked via the trimetazidine moiety.

### Isotopically-Enriched Compounds

The compounds may include one or more atoms that are enriched for an isotope. For example, the compounds may have one or more hydrogen atoms replaced with deuterium or tritium. Isotopic substitution or enrichment may occur at carbon, sulfur, or phosphorus, or other atoms. The compounds may be isotopically substituted or enriched for a given atom at one or more positions within the compound, or the compounds may be isotopically substituted or enriched at all instances of a given atom within the compound.

### Compositions

In certain embodiments, methods of the invention include providing pharmaceutical compositions containing one or more of the compounds described above. A pharmaceutical composition containing a compound may be in a form suitable for oral use, for example, as tablets, troches, lozenges, fast-melts, aqueous or oily suspensions, dispersible powders or granules, emulsions, hard or soft capsules, syrups or elixirs. Compositions intended for oral use may be prepared according to any method known in the art for the manufacture of pharmaceutical compositions and such compositions may contain one or more agents selected from sweetening agents, flavoring agents, coloring agents and preserving agents, in order to provide pharmaceutically elegant and palatable preparations. Tablets contain the compounds in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch, or alginic acid; binding agents, for example starch, gelatin or acacia, and lubricating agents, for example magnesium stearate, stearic acid or talc. Preparation and administration of compounds is discussed in U.S. Pat. No. 6,214,841 and U.S. Patent Publication No. 2003/0232877, the contents of each of which are incorporated by reference herein.

Formulations for oral use may also be presented as hard gelatin capsules in which the compounds are mixed with an inert solid diluent, for example calcium carbonate, calcium phosphate or kaolin, or as soft gelatin capsules in which the compounds are mixed with water or an oil medium, for example peanut oil, liquid paraffin or olive oil.

Aqueous suspensions may contain the compounds in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents such as a naturally occurring phosphatide, for example lecithin, or condensation products of an alkylene oxide with fatty acids, for example, polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such a polyoxyethylene with partial esters derived from fatty acids and hexitol anhydrides, for example polyoxyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the compounds in a vegetable oil, for example, arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the compounds in admixture with a dispersing or wetting agent, suspending agent and one or more preservatives. Suitable dispersing or wetting agents and suspending agents are exemplified, for example sweetening, flavoring and coloring agents, may also be present.

The pharmaceutical compositions use in methods of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally occurring phosphatides, for example soya bean, lecithin, and esters or partial esters derived from fatty acids and hexitol anhydrides, for example sorbitan monooleate and condensation products of the said partial esters with ethylene oxide, for example polyoxyethylene sorbitan monooleate. The emulsions may also contain sweetening and flavoring agents.

Syrups and elixirs may be formulated with sweetening agents, such as glycerol, propylene glycol, sorbitol or sucrose. Such formulations may also contain a demulcent, a preservative, and agents for flavoring and/or coloring. The pharmaceutical compositions may be in the form of a sterile injectable aqueous or oleaginous suspension. This suspension may be formulated according to the known art using those suitable dispersing or wetting agents and suspending agents which have been mentioned above. The sterile injectable preparation may also be in a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid find use in the preparation of injectables.

### Increasing Cardiac Efficiency

The compounds of the invention are useful for improving cardiac efficiency. A variety of definitions of cardiac efficiency exist in the medical literature. See, e.g., Schipke, J. D. Cardiac efficiency, Basic Res. Cardiol. 89:207-40 (1994); and Gibbs, C. L. and Barclay, C. J. Cardiac efficiency, Cardiovasc. Res. 30:627-634 (1995), incorporated herein by reference. One definition of cardiac mechanical efficiency is the ratio of external cardiac power to cardiac energy expenditure by the left ventricle. See Lopaschuk G. D., et al., Myocardial Fatty Acid Metabolism in Health and Disease, Phys. Rev. 90:207-258 (2010), incorporated herein by reference. Another definition is the ratio between stroke work and oxygen consumption, which ranges from 20-25% in the normal human heart. Visser, F., Measuring cardiac efficiency: is it useful? Hear Metab. 39:3-4 (2008), incorporated herein by reference. Another definition is the ratio of the stroke volume to mean arterial blood pressure. Any suitable definition of cardiac efficiency may be used to measure the effects of compounds of the invention.

### Treating Cardiac Steatosis and Reducing Myocardial Triglycerides

The method of the invention is useful for treating cardiac steatosis or a disorder associated with cardiac steatosis. Cardiac steatosis is ectopic deposition and abnormal retention of lipids within the heart. It is visualized histologically as fatty acid droplets within the sarcolemma and can be quantified using non-invasive imaging of myocardial triglyceride (MTG) using ¹H-magnetic resonance spectroscopy (MRS), as discussed in Szczepaniak L S, Victor R G, Orci L, Unger R H. Forgotten but not gone: the rediscovery of fatty heart, the most common unrecognized disease in America. Circ Res. 2007 Oct. 12; 101 (8): 759-67. doi: 10.1161/circresaha. 107.160457. PMID: 17932333, the entirety of the contents of which are incorporated by reference herein. Cardiac steatosis is commonly associated with diabetes and/or obesity where it is thought to reflect excessive free fatty acid delivery to the heart. This process of excessive ectopic lipid deposition (steatosis) may also affect other non-adipose organs (e.g. liver, contributing to non-alcoholic fatty liver disease or hepatic steatosis, pancreas) as well as the epicardium and pericardium. In humans, impaired glucose tolerance is accompanied by cardiac steatosis, which precedes the onset of type 2 diabetes mellitus and left ventricular systolic dysfunction. Lipid overstorage in human cardiac myocytes is an early manifestation in the pathogenesis of type 2 diabetes mellitus and is evident in the absence of heart failure. Jonathan M. McGavock. Circulation. Cardiac Steatosis in Diabetes, Mellitus, Volume: 116, Issue: 10, Pages: 1170-1175, DOI:(10.1161/circulationaha.106.645614). Cardiac steatosis reflects an impairment of the normal processes of synthesis and elimination of triglyceride fat. Hydrolysis of excessive myocardial cytosolic triglyceride stores expands the cellular free fatty acid pool, providing substrate for harmful cellular fatty acid pathways such as ceramide which can induce apoptosis in cardiomyocytes (and pancreatic β cells) and promote insulin resistance. The activation of these adverse signaling cascades secondary to by-products of lipid metabolism, such as ceramide or other fatty acid derivatives, leading to cell death is termed lipotoxicity, as discussed in McGavock J M, Victor R G, Unger R H, Szczepaniak L S; American College of Physicians and the American Physiological Society. Adiposity of the heart, revisited. Ann Intern Med. 2006 Apr. 4; 144 (7): 517-24. doi: 10.7326/0003-4819-144-7-200604040-00011. PMID: 16585666, the entirety of the contents of which are incorporated by reference herein. Examination of transplanted hearts from non-diabetic individuals in patients with diabetes shows that cardiomyocyte lipid accumulation (including triglyceride and ceramide) is an early and progressive pathological event in the context of a diabetic milieu and worsening of donor cardiac function at 48 weeks post-transplant, as discussed in Marfella R, Amarelli C, Cacciatore F, Balestrieri M L, Mansueto G, D'Onofrio N, Esposito S, Mattucci I, Salerno G, De Feo M, D'Amico M, Golino P, Maiello C, Paolisso G, Napoli C. Lipid Accumulation in Hearts Transplanted From Nondiabetic Donors to Diabetic Recipients. J Am Coll Cardiol. 2020 Mar. 24; 75 (11): 1249-1262. doi: 10.1016/j.jacc.2020.01.018. PMID: 32192650, the entirety of the contents of which are incorporated by reference herein. Myocardial triglyceride content is independently associated with impaired left ventricle diastolic function in Type 2 diabetes, (Rijzewijk et al, JACC 2008), and with greater impairment of right ventricle and left ventricle strain. (Ng et al, Circ 2010).

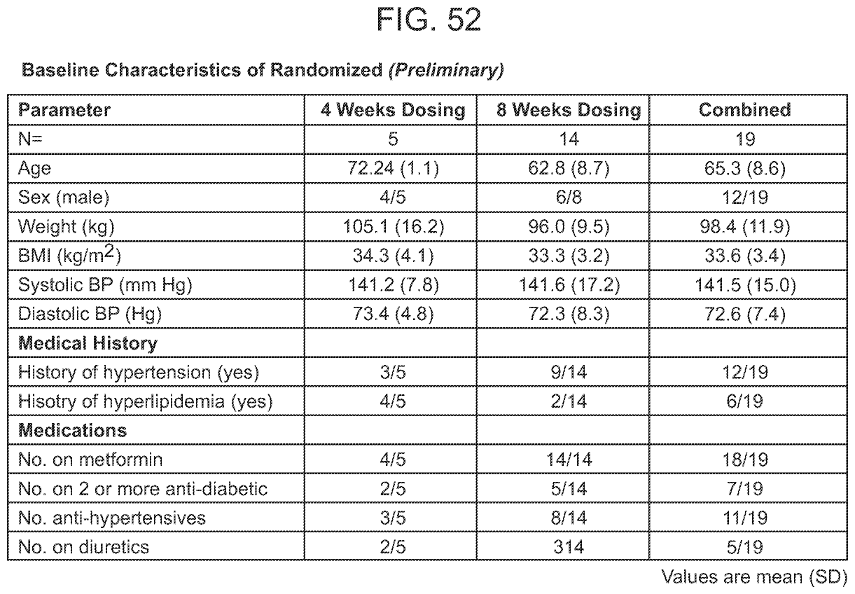

Results of a pharmacodynamic study to evaluate the impact of 200 mg BID of a composition of a compound with formula (X) on rest myocardial energetics (PCr/ATP) in obese individuals with type 2 diabetes are detailed in FIGS. 52-65. The key endpoints of the study were to evaluate the metabolic response, specifically the impact on PDH flux using hyperpolarized ¹³C-pyruvate MRS, as a measure of the compound's ability to promote glucose oxidation and recouple glucose oxidation with glycolysis. Also assessed was the effect on cardiac systolic and diastolic function, measured by cardiac magnetic resonance (CMR) and transthoracic echocardiography (TTE), and the effect on cardiac steatosis using ¹H-MRS.

The method of the invention provides a robust and rapid impact on myocardial steatosis, i.e. reducing myocardial lipid overload, to support better function of the obese and/or diabetic myocardium. The method includes providing to a subject having, or at risk of developing, cardiac steatosis or a disorder associated with cardiac steatosis a composition of a compound having a structure represented by formula (X). In the method, the composition may be provided orally. The composition may be provided in at least one dose per day or may be provided in multiple doses per day at a suitable interval. As an example, the composition may be provided in at least one dose daily for at least two weeks. The dose of the compound of formula (X) may be from about 25 mg to about 1000 mg, from about 50 mg to about 600 mg, and from about 100 mg to about 400 mg. Preferably, the dose may be about 200 mg. The composition may be a modified-release formulation.

The methods of the invention are useful for reducing myocardial triglycerides in a subject. The method includes providing to a subject having, or at risk of developing, myocardial a composition of a compound having a structure represented by formula (X). In the method, the composition may be provided orally. The composition may be provided in at least one dose per day or may be provided in multiple doses per day at a suitable interval. As an example, the composition may be provided in at least one dose daily for at least two weeks. The dose of the compound of formula (X) may be from about 25 mg to about 1000 mg, from about 50 mg to about 600 mg, and from about 100 mg to about 400 mg. Preferably, the dose may be about 200 mg. The composition may be a modified-release formulation.

### Reducing Lipotoxicity

Intracellular triglycerides can be metabolized to toxic by-products (e.g. diacylglycerol and ceramides) in a process termed liptoxicity. Cardiac lipotoxicity not only involves an excessive accumulation of intra-myocellular triglycerides (TGs) in the heart but also changes in lipid classes, as well as in their fatty acid profile. The method includes providing to a subject having, or at risk of developing, lipotoxicity a composition of a compound having a structure represented by formula (X). In the method, the composition may be provided orally. The composition may be provided in at least one dose per day or may be provided in multiple doses per day at a suitable interval. As an example, the composition may be provided in at least one dose daily for at least two weeks. The dose of the compound of formula (X) may be from about 25 mg to about 1000 mg, from about 50 mg to about 600 mg, and from about 100 mg to about 400 mg. Preferably, the dose may be about 200 mg. The composition may be a modified-release formulation.

### Inducing Weight Loss

Even short-term administration of formula (X) is associated with meaningful and rapid systemic metabolic effects. See example 3. The majority of subjects in the study lost weight with greater weight loss in those with higher baseline HbA1c. A significant reduction in mean fasting glucose in the cohorts was also achieved.

### As a Predictor of Cardiac Energetic Response

Methods of the invention are useful for treating cardiac dysfunction. Baseline HbA1c and reduction of HbA1c using the methods of this invention are positively associated with an increase in PCr/ATP. The method includes providing to a subject having an elevated level of HbA1c at least one dose per day of a compound having a structure represented by formula (X). A cutoff baseline HbA1c of greater than 6.0%, 6.5% or 7% may be used as a predictor of PCr/ATP response.

### EXAMPLES

### Example 1

### Brief Study Design

A Phase 1 first-in-human, randomized, double-blind, placebo-controlled single ascending dose and multiple ascending dose study to investigate the safety, tolerability, and pharmacokinetics (including food effect) or IMB-1018972 in healthy subjects.

### Objectives

The primary objective is to assess the safety and tolerability of single and multiple ascending oral doses of IMB-1018972, and single oral doses of trimetazidine.

Secondary objectives include: To assess the pharmacokinetic (PK) profile of single and multiple ascending oral doses of IMB-1018972, and single oral doses of trimetazidine; To assess the effect of food on the absorption and the PK profile of IMB-1018972 following a single oral dose of IMB-1018972 in healthy subjects; To evaluate the effect of food on the safety and tolerability of IMB-1018972 following a single oral dose of IMB-1018972 in healthy subjects;

### Design and Treatments

This was a double-blind, randomized, placebo-controlled study, consisting of a single ascending dose (SAD) part with integrated food effect (FE) arm, a multiple ascending dose (MAD) part, to assess the safety, tolerability, and PK of ascending single and multiple oral doses of IMB-1018972 (immediate-release [IR] formulation in the SAD and MAD parts). The study started with the SAD part.

### SAD Part (and Integrated FE Arm)

In the SAD part, 5 groups of 8 healthy subjects (6 subjects on active drug and 2 on placebo in Groups A1, A2, A3, and A4, and 8 subjects on active drug in Group A5) were included. In Groups A1, A2, A3, and A4, subjects received a single oral dose of an IR formulation of IMB-1018972 or placebo under fasted conditions (an overnight fast of at least 10 hours). In Group A5, all subjects received a single oral dose of a MR formulation of trimetazidine under fasted conditions (an overnight fast of at least 10 hours). Each subject participated in only 1 group during the study.

Subjects assigned to Group A4 also participated in the FE arm and received the same single dose of IMB-1018972 or placebo under fed conditions (Food and Drug Administration [FDA]-defined high-fat breakfast after an overnight fast of at least 10 hours) in a second period at least 1 week after drug administration under fasted conditions in the SAD part.

The following treatments were administered in the SAD part under fasted conditions:
Group A1: single oral dose of 50 mg IR formulation of IMB-1018972 (n=6) or matching placebo (n=2) on Day 1
Group A2: single oral dose of 150 mg IR formulation of IMB-1018972 (n=6) or matching placebo (n=2) on Day 1
Group A3: single oral dose of 400 mg IR formulation of IMB-1018972 (n=6) or matching placebo (n=2) on Day 1
Group A4: single oral dose of 150 mg IR formulation of IMB-1018972 (n=6) or matching placebo (n=2) on Day 1 (FE group)
Group A5: single oral dose of 35 mg MR formulation of trimetazidine (Vastarel; n=8) on Day 1
The following treatment was administered in the FE arm under fed conditions (FDA-defined high-fat breakfast):
Group A4: single oral dose of 150 mg IR formulation of IMB-1018972 (n=5) or matching placebo (n=2) on Day 1 (same dose as in SAD part)

IMB-1018972 dose-escalation was based on the available safety, tolerability, and PK results of at least 5 dosed subjects in the preceding group. A dose-escalation meeting was held between the Investigator and the Sponsor. Further, a dose-escalation report (DER) was provided by the Investigator to the Independent Ethics Committee (IEC) following completion of each dose level. Escalation to the next higher dose only proceeded when none of the stopping criteria had been reached and if the available safety, tolerability, and PK results (results up to 48 hours postdose) of at least 5 dosed subjects in the preceding group were acceptable to the Investigator and the Sponsor and after a statement of no objection of the DER from the IEC.

In this first-in-human (FIH) study, the subjects participating at the lowest dose level, subjects of Group A1, were dosed according to a sentinel dosing design to ensure optimal safety. This means that initially, 2 subjects were dosed: 1 subject with IMB-1018972 and 1 subject with placebo. Since the safety and tolerability results of the first 24 hours following dosing for the initial 2 subjects were acceptable to the Investigator, the other 6 subjects (5 active drug and 1 placebo) of the lowest dose level were also dosed.

### MAD Part

In the MAD part, 2 groups of 12 healthy subjects (9 subjects on active drug and 3 on placebo in each group) were included. Subjects received multiple oral doses of an IR formulation of IMB-1018972 or placebo once every 12 hours (q12 h) for 14 consecutive days. Each subject participated in only 1 group during the study.

The following treatments were administered under fed conditions as determined based on the results of Group A4 in the FE arm. The doses were selected based upon the safety, tolerability, and PK data from the SAD part:
Group B1 multiple oral doses of an IR formulation of 150 mg IMB-1018972 (n=9) or matching placebo (n=3) twice daily (q12 h) for 14 days; on Day 14 only a single morning dose was administered
Group B2: multiple oral doses of an IR formulation of 50 mg IMB-1018972 (n=9) or matching placebo (n=3) q12 h for 14 days; on Day 14 only a single morning dose was administered

IMB-1018972 dose escalation was based on the available safety, tolerability, and PK results of at least 8 dosed subjects in the preceding group. A dose-escalation meeting was held between the Investigator and the Sponsor. Further, a DER was provided by the Investigator to the IEC following completion of each dose level. Escalation to the next higher dose only proceed when none of the stopping criteria had been reached and if the available safety, tolerability, and PK results (results up to 48 hours after the final morning dose on Day 14) of at least 8 dosed subjects in the preceding group were acceptable to the Investigator and the Sponsor and after a statement of no objection of the DER from the IEC.

### Study Schedule

Screening: Between Day −35 and Day −1 (admission)
Confinement period: SAD part: 1 period in the clinic from Day −1 (admission) to approximately 48 hours after study drug administration (Day 3); an exception was Group A4 also participating in the FE arm in which subjects were in the clinic for 2 periods, each being from Day −1 (admission) to approximately 48 hours after study drug administration (Day 3) MAD part: 1 period in the clinic from Day −1 (admission) to approximately 48 hours after the last study drug administration on Day 14 (Day 16);
Follow-up: SAD part: 7 to 14 days after the last PK blood sample (between Day 10 and Day 17); FE arm: 7 to 14 days after the last PK blood sample in the second period (between Day 10 and Day 17); MAD part: 7 to 14 days after the last PK blood sample (between Day 23 and Day 30);

### Subjects

SAD part: 40 healthy male or female subjects (this included 8 subjects also participating in the FE arm); from Group A4 onwards, all efforts were made to have a ratio of 50:50 for male and female subjects per group, but at minimum at least 3 subjects of each gender were dosed per group
MAD part: 24 healthy male or female subjects; for each group, all efforts were made to have a ratio of 50:50 for male and female subjects, but at minimum at least 4 subjects of each gender were dosed per group

### Main Criteria for Inclusion

Age: 18 years to 65 years, inclusive, at screening
Body mass index (BMI): 18.0 kg/m² to 32.0 kg/m², inclusive
Status: Healthy subjects

### Study Drug

### Active Medication

Drug product: IMB-1018972
Activity: Fatty acid oxidation inhibitor
In development for: Ischemic cardiovascular disease
Strength: 25 mg, 100 mg, and 200 mg IR formulations (based on free base)
Dosage form: Oral IR capsule(s) to be used in the SAD and MAD parts
Manufacturer: Pharmacy at PRA
Batch number: 2479-1810-00441 (drug substance)
   IMB-1018972 placebo (visually matching active medication)
Active substance: Not applicable
Activity: Not applicable
Strength: Not applicable
Dosage form: Oral capsule(s)
Manufacturer: Pharmacy at PRA
Batch number: Not applicable

### Active Medication

Drug product: Vastarel MR (trimetazidine dihydrochloride)
Activity: Fatty acid oxidation inhibitor
In development for: Angina pectoris
Strength: 35 mg
Dosage form: Oral modified-release tablet
Manufacturer: Servier Research & Pharmaceuticals (Pakistan) (Pvt.) Ltd.
Batch number: 273782 (drug product)

### Variables

Safety: Adverse events, clinical laboratory, vital signs, 12-lead electrocardiogram, continuous cardiac monitoring (telemetry), and physical examination
Plasma concentrations of IMB-1018972, IMB-1028814, and trimetazidine Pharmacokinetics: Urine concentrations of IMB-1018972, IMB-1028814, and trimetazidine (SAD part only) Plasma PK parameters estimated using noncompartmental analysis, as appropriate. SAD part, integrated FE arm: Cₘₐₓ, tₘₐₓ, AUC₀₋ₜ, AUC_{0-inf}, % AUCₑₓₜᵣₐ, kₑₗ, t_{1/2}, CL/F (IMB-1028814 only), and V_{z}/F (IMB-1028814 only). Day 1 of MAD part: Cₘₐₓ, tₘₐₓ, and AUC_{0-T}. Day 14 of MAD part: Cₘₐₓ, tₘₐₓ, Cₘᵢₙ, kₑₗ, t_{1/2}, AUC_{0-T}, CLₛₛ/F (IMB-1028814 only), V_{z}/F (IMB-1028814 only), and R_{ac} Urine PK parameters estimated using noncompartmental analysis, as appropriate: Aeᵤᵣᵢₙₑ, Feᵤᵣᵢₙₑ, and CL_{R}

### Statistical Methods

Sample size calculation: For this FIH study, no prospective calculations of statistical power were made. The sample size was selected to provide information on safety, tolerability, and PK following single and multiple doses of IMB-1018972, single doses of trimetazidine, and is typical for a FIH study. Any p-values to be calculated according to the statistical analysis plan were interpreted in the perspective of the exploratory character of this study.
Safety parameters: Descriptive statistics
PK parameters: Descriptive statistics for all relevant PK parameters: n, mean, SD, minimum, median, maximum, geometric mean, and coefficient of variation; analysis of variance on Cₘₐₓ and AUC parameters to determine dose proportionality and FE

### Results

### Subject Disposition

Of the 220 subjects who were screened, 88 subjects were included in the study and received the study drug. Sixty-six subjects received a dose of IMB-1018972, 8 received trimetazidine, and 14 received placebo. Eighty-five of 88 subjects completed the study. One subject of the FE arm Group A4 withdrew consent on Day 1 of the second period after receiving the single oral dose of 150 mg IMB-1018972 under fed conditions. Another subject of the FE arm Group A4 was withdrawn from the study due to a serious adverse event (SAE) of influenza like illness (of moderate severity and unlikely related) in the first period and only received the single oral dose of 150 mg IMB-1018972 under fasted conditions and not the fed dose in the second treatment period. None of these discontinued subjects were replaced. All 88 subjects were included in the PK and safety sets.

TABLE 1
Disposition of Subject

| | Number of subjects |
|---|---|
| Screened volunteers | 220 |
| Screening failures | 78 |
| Approved but not receiving study drug | 54 |
| Reserve | 24 |
| Group full | 9 |
| Personal reasons | 9 |
| Group cancelled | 8 |
| Rejected in clinic | 3 |
| Illness of volunteer | 1 |
| Subjects receiving at least 1 dose of study drug | 88 |
| Any dose of IMB-1018972 | 66 |
| Placebo dose | 14 |
| Trimetazidine dose | 8 |
| Discontinued subjects | |
| Adverse event | 2 |
| Withdrawal by subject | 1 |
| Completed subjects | 85 |

### Demographics

### SAD Part (and Integrated FE Arm)

### IMB-10108972 and Placebo

Thirty-two subjects were included of whom 23 were female and 9 were male. Mean age ranged between 29 and 46 years and mean BMI ranged between 23.0 and 26.6 kg/m2 over all treatments, including placebo. Individual age ranged between 18 and 65 years and individual BMI ranged between 19.5 and 30.3 kg/m2. Twenty-nine subjects were of white race, 1 subject was Asian, 1 subject was Black or African American, and 1 subject was Native Hawaiian or Other Pacific Islander.

### Trimetazidine Group

Eight subjects were included of whom 5 were female and 3 were male. Mean age was 32 years and mean BMI was 23.7 kg/m2. Individual age ranged between 20 and 65 years and individual BMI ranged between 19.4 and 26.7 kg/m2. Seven subjects were of white race and 1 subject was of multiple race.

### MAD Part

Twenty-four subjects were included of whom 12 were female and 12 were male. Mean age ranged between 38 and 44 years and mean BMI ranged between 25.2 and 26.7 kg/m2 over all treatments, including placebo. Individual age ranged between 18 and 64 years and individual BMI ranged between 19.1 and 30.9 kg/m2. Eighteen subjects were of white race, 2 subjects were of multiple race, 2 subjects were American Indian or Alaska Native, 1 subject was Asian, and 1 subject was Black or African American.

### Safety

In the SAD part, treatment with single oral doses of 50 mg, 150 mg, and 400 mg IMB-1018972 under fasted conditions, treatment with single oral doses of 150 mg IMB-1018972 under fed conditions, and treatment with single oral doses of 35 mg trimetazidine were well tolerated by healthy male and female subjects. During the SAD part, the most common AEs were 6 TEAEs of flushing (reported terms were ‘niacin flush’ and ‘flushing neck’), of which 5 TEAEs were of moderate severity and 1 TEAE was of mild severity. Four subjects reported flushing after a single dose of 400 mg IMB-1018972 under fasted conditions, and 2 subjects of the FE arm reported flushing after a single dose of 150 mg IMB-1018972 under fasted conditions. These TEAEs were all considered by the Investigator to be related to the study drug. No subjects dropped out due to flushing and flushing was not considered a safety issue. There were no clinically important trends in the physical examinations, vital signs, clinical laboratory, or ECG results. Dose escalation beyond 400 mg IMB-1018972 IR did not proceed as planned based on the PK exposure levels of IMB-1028814 and trimetazidine exceeding the target exposure levels in the 400 mg group and the findings of flushing at that dose. The predefined target exposure level was approximately 3 to 4 ‘trimetazidine equivalents’, ie, the ratio of the combined exposure of the active metabolites of IMB-1018972 to the single oral doses of 35 mg MR trimetazidine as seen in published literature.

In the MAD part, 14-day treatment with oral q12 h doses of 50 mg and 150 mg IMB-1018972 under fed conditions was well tolerated by healthy male and female subjects. The most common AEs were 7 incidental and mild TEAEs of flushing that occurred in 6 subjects who had received 150 mg IMB-1018972 q12 h. Five of these 6 subjects reported only a single TEAE of flushing during the 14 days dosing period. One subject reported flushing twice, on Day 2 and on Day 14. No TEAEs of flushing were reported following administration of 50 mg IMB-1018972 q12 h. No subjects dropped out and no modification of the dose was needed due to the TEAEs of flushing.

Overall, no deaths were reported during the study. The majority of the reported TEAEs were transient and resolved without sequelae by follow-up. Most TEAEs were of mild severity and no severe TEAEs were reported during the study. TEAEs of moderate severity were the 5 TEAEs of flushing mentioned above and 1 TEAE each of restlessness, back pain, nausea, tonsillitis, post procedural hemorrhage, ALT increased, and influenza like illness. The moderate TEAE of influenza like illness was considered to be an SAE and was reported by a subject in the SAD part who had received a single dose of 150 mg IMB-1018972 under fasted conditions in the SAD part. The subject was withdrawn from the study as a result of this SAE. The SAE was considered by the Investigator unlikely to be related to the study drug.

In both the SAD part and MAD part, there was no clear dose dependency of the number and incidence of TEAEs. In the FE arm of the SAD part, dosing under fed conditions appeared to attenuate the number and incidence of TEAEs.

The most frequently reported TEAEs during the study were of the system organ class vascular disorders (mainly TEAEs of flushing), general disorders and administration site conditions, nervous system disorders, gastrointestinal disorders, and musculoskeletal and connective tissue disorders.

The majority of the TEAEs reported during the study were considered by the Investigator not to be related to the study drug.

There were no findings of clinical relevance with respect to clinical laboratory, vital signs, 12-lead ECG, continuous cardiac monitoring (telemetry), or physical examination.

### Pharmacokinetics

All blood samples of subjects that received IMB-1018972 in this study were analyzed for IMB-1018972 in plasma, but IMB-1018972 could be measured in only few plasma samples. Therefore, the IMB-1018972 concentrations have only been listed and no descriptive statistics or concentration-time profiles have been presented in this clinical study report. In addition, no PK parameters have been calculated for plasma IMB-1018972. As a result, urine samples were not analyzed for IMB-1018972 concentrations.

Since the pharmacodynamic effect of IMB-1028814 and trimetazidine is the same, data are presented for IMB-1028814 and trimetazidine individually, as well as for the sum of IMB-1028814 and trimetazidine concentrations.

### SAD Part (and Integrated FE Arm)

### PK in Plasma Following Administration of IMB-1018972 Under Fasted Conditions

The initial hydrolysis of IMB-1018972 to IMB-1028814 and subsequent systemic bioavailability of IMB-1028814 was relatively rapid with median tₘₐₓ around 1 hour postdose for IMB-1028814, and between 1.5 hours and 2 hours postdose for trimetazidine over the studied single-dose range of 50 mg to 400 mg IMB-1018972 under fasted conditions. Median tₘₐₓ did not increase with increasing IMB-1018972 dose.

The geometric mean Cₘₐₓ increased with dose and ranged between 104 ng/ml and 870 ng/mL for IMB-1028814, between 36.9 ng/ml and 274 ng/mL for trimetazidine, and between 516 nmol/L and 3,839 nmol/L (molar units to account for differences in molecular weight) for IMB-1028814+trimetazidine over the studied single-dose range of 50 mg to 400 mg IMB-1018972 under fasted conditions. Similarly, the geometric mean AUC₀₋ₜ increased with dose and ranged between 290 ng·h/mL and 2,795 ng·h/mL for IMB-1028814, between 424 ng·h/mL and 3,305 ng·h/mL for trimetazidine, and between 2,970 nmol·h/L and 22,365 nmol·h/L for IMB-1028814+trimetazidine over the studied single-dose range of 50 mg to 400 mg IMB-1018972 under fasted conditions. The predefined stopping criterion for IMB-1028814 plasma exposure of 417,733 and 652,849 ng·h/mL for males and females, respectively, was not reached by any of the subjects during the SAD part.

Dose proportionality of IMB-1028814 and trimetazidine was explored for Cₘₐₓ, AUC0-t, and AUC0-inf. The 95% CIs of the slopes of all 3 exposure parameters included 1 for both IMB-1028814 and trimetazidine indicating no evidence of a deviation from dose proportionality of IMB-1028814 and trimetazidine over the IMB-1018972 single-dose range of 50 to 400 mg under fasted conditions.

The geometric mean t_{1/2} of IMB-1028814 was relatively short, ranging between 2.6 hours and 3 hours over the IMB-1018972 single-dose range under fasted conditions. For metabolite trimetazidine, geometric mean t_{1/2} was longer, ranging between 6.76 hours and 8 hours over the IMB-1018972 single-dose range under fasted conditions. Geometric mean t_{1/2} of IMB-1028814 and trimetazidine did not increase with increasing IMB-1018972 dose indicating that the PK of the 2 moieties was linear.

### PK in Plasma Following Administration of Trimetazidine

Following administration of a single oral dose of 35 mg trimetazidine, median trimetazidine tmax was 5 hours, and geometric mean values were 68.6 ng/mL for Cmax, 912 ng·h/mL for AUC₀₋ₜ, and 929 ng·h/mL for AUC_{0-inf}. The geometric mean t1/2 of trimetazidine was 7.49 hours.

### Effect of Food

The possible effect of food on the PK of IMB-1028814 and trimetazidine was explored by comparing administration of single oral doses of 150 mg IMB-1018972 after an FDA-defined high-fat breakfast and under fasted conditions.

Median IMB-1028814 tₘₐₓ in plasma was reached at 2 hours postdose under fed conditions relative to 1 hour postdose under fasted conditions. Median trimetazidine tₘₐₓ in plasma was reached at 4 hours postdose under fed conditions relative to 1.5 hours postdose under fasted conditions.

The effect of food of IMB-1028814 and trimetazidine was explored for Cₘₐₓ, AUC0-t, and AUC_{0-inf}. No evidence for an effect of food was observed on the IMB-1028814 exposure parameters AUC₀₋ₜ and AUC_{0-inf} (both with an estimate of 1.12 and 90% CI ranging from 1.02 to 1.23). However, Cₘₐₓ was approximately 36% lower following administration of a single dose of 150 mg IMB-1018972 after an FDA-defined high-fat breakfast relative to administration under fasted conditions (estimate of 0.64; 90% CI ranging from 0.39 to 1.04).

No evidence for an effect of food was observed on the trimetazidine exposure parameters Cₘₐₓ (estimate of 0.91; 90% CI ranging from 0.85 to 0.98), and AUC0-t and AUC0-inf (both with an estimate of 1.04 and 90% CI ranging from 0.98 to 1.10) following administration of a single dose of 150 mg IMB-1018972.

### PK in Urine

The arithmetic mean percent of the dose excreted in urine ranged between 3.99% and 5.74% for IMB-1028814, and between 23.11% and 32.55% for trimetazidine within 48 hours after a single oral IMB-1018972 dose over the studied dose range of 50 mg to 400 mg. Within 48 hours following administration of a single oral dose of 35 mg trimetazidine, an arithmetic mean of 54.47% was excreted in urine as trimetazidine. These results indicate that metabolism is the primary clearance mechanism for IMB-1028814 while renal excretion is the primary clearance mechanism for trimetazidine.

The geometric mean renal clearance (CLR) ranged between 3.76 L/h and 5.37 L/h for IMB-1028814, and between 18.1 L/h and 20.8 L/h for trimetazidine over the studied single-dose range of 50 mg to 400 mg IMB-1018972. Geometric mean CLR for trimetazidine was 20.4 L/h following administration of a single oral dose of 35 mg trimetazidine. The renal clearance of trimetazidine is greater than the glomerular filtration rate (125 mL/min or 7.5 L/h), indicating that trimetazidine undergoes net tubular secretion.

### MAD Part

Over the 2 multiple-dose levels, median tₘₐₓ ranged between 0.5 hours and 1 hours postdose for IMB-1028814 on Day 1, and was 3 hours postdose for trimetazidine on Day 1. On Day 14, median tₘₐₓ was 0.5 hours postdose for IMB-1028814 and 2 hours postdose for trimetazidine.

### Exposure Parameters on Day 1

No dose-proportionality analysis was done since there were only 2 IMB-1018972 dose levels in the MAD part: multiple oral doses of 50 mg or 150 mg q12 h for 14 days under fed conditions.

The geometric mean Cₘₐₓ and AUC0-T were higher after 150 mg fed than after 50 mg fed for IMB-1028814 (297% and 336% higher for Cₘₐₓ and AUC_{0-T}, respectively), trimetazidine (154% and 163% higher for Cₘₐₓ and AUC_{0-T} respectively), and IMB-1028814+trimetazidine (257% and 239% higher for Cₘₐₓ and AUC_{0-T}, respectively).

When comparing the MAD and SAD parts, geometric mean Cₘₐₓ was 97% higher on Day 1 after 150 mg fed in the MAD part than after a single dose of 150 mg fed in the SAD part for IMB-1028814. For trimetazidine however, geometric mean Cₘₐₓ was 32% lower on Day 1 after 150 mg fed in the MAD part than after a single dose of 150 mg fed in the SAD part.

### Exposure Parameters on Day 14 Following Repeated q12 h Dosing

The geometric mean Cₘₐₓ and AUC_{0-T} were higher after 150 mg fed than after 50 mg fed for IMB-1028814 (377% and 367% higher for Cₘₐₓ and AUC_{0-T}, respectively), trimetazidine (127% and 126% higher for Cₘₐₓ and AUC_{0-T}, respectively), and IMB-1028814+trimetazidine (286% and 211% higher for Cₘₐₓ and AUC_{0-T}, respectively).

The predefined stopping criterion for IMB-1028814 plasma exposure of 417,733 and 652,849 ng·h/mL for males and females, respectively, was not reached by any of the subjects during the MAD part.

### Trough Concentrations Following Repeated q12 h Dosing

Based upon visual inspection of the geometric mean plasma concentration-time profiles and the geometric mean trough concentrations, it can be concluded that for both 150 mg fed and 50 mg fed, the Day 14 IMB-1018972 dose was administered under steady-state conditions of IMB-1028814 and trimetazidine concentrations.

### Accumulation Following Repeated q12 h Dosing

For both the 50 mg and 150 mg fed dose levels, geometric mean AUC_{0-T} values of IMB-1028814, trimetazidine, and IMB-1028814+trimetazidine were higher on Day 14 relative to Day 1. Geometric mean Rac for IMB-1028814 was 1.18 and 1.10 after the 150 mg fed dose and 50 mg fed dose, respectively, indicating minimal accumulation of IMB-1028814 in plasma. Geometric mean Rac for trimetazidine was 1.63 and 1.89 after the 150 mg fed dose and 50 mg fed dose, respectively, indicating modest accumulation of trimetazidine in plasma. Geometric mean Rac for IMB-1028814+trimetazidine was 1.39 and 1.52 after the 150 mg fed dose and 50 mg fed dose, respectively, indicating modest accumulation of IMB-1028814+trimetazidine in plasma.

### Terminal Elimination Half-Life Following Repeated q12 h Dosing

For IMB-1028814, the geometric mean t1/2 of 4.48 hours after 150 mg fed was longer than that of 2.79 hours after 50 mg fed. For trimetazidine, the geometric mean t1/2 of 9.36 hours after 150 mg fed was similar to that of 9.32 hours after 50 mg fed. For IMB-1028814+trimetazidine, the geometric mean t_{1/2} of 8.90 hours for IMB-1028814 after 150 mg fed was similar to that of 9.08 hours after 50 mg fed.

### Conclusions

### Safety

Overall, single oral IMB-1018972 doses and multiple oral IMB-1018972 doses of an IR formulation were generally well tolerated by healthy male and female subjects. There were no findings of clinical relevance with respect to clinical laboratory, vital signs, 12-lead ECG, continuous cardiac monitoring (telemetry), or physical examination. Of note, there were no findings of hemodynamic changes, nor changes in the QTc-interval, after administration of IMB-1018972 as the IR.
During the SAD part, the most common AEs were 6 TEAEs of flushing (reported terms were ‘niacin flush’ and ‘flushing neck’), of which 5 TEAEs were of moderate severity and 1 TEAE was of mild severity. Four subjects reported flushing after a single dose of 400 mg IMB-1018972 under fasted conditions, and 2 subjects of the FE arm reported flushing after a single dose of 150 mg IMB-1018972 under fasted conditions. These TEAEs were all considered by the Investigator to be related to the study drug. No subjects dropped out due to flushing and flushing was not considered a safety issue. Dose escalation beyond 400 mg IMB-1018972 IR did not proceed as planned based on the PK exposure levels of IMB-1028814 and trimetazidine exceeding the target exposure levels in the 400 mg group and the findings of flushing at that dose. The predefined target exposure level was approximately 3 to 4 ‘trimetazidine equivalents’, ie, the ratio of the combined exposure of the active metabolites of IMB-1018972 to the single oral doses of 35 mg MR trimetazidine as seen in published literature.
There were no deaths reported during the study. Most TEAEs were of mild severity and no severe TEAEs were reported during the study. Overall, 12 of a total of 181 TEAEs were of moderate severity.
Two subjects were withdrawn from the study: 1 subject due to a moderate SAE of influenza like illness (unlikely related) and 1 due to a moderate TEAE of ALT increased (possibly related).
Overall, there was no clear dose dependency of the number and incidence of TEAEs.
Dosing under fed conditions appeared to attenuate the number and incidence of TEAEs in the FE arm of the SAD part.

### Pharmacokinetics

IMB-1018972 could be measured in only few plasma samples taken during this study.
When combining the single and multiple IMB-1018972 dose results under fasted and fed conditions, the initial hydrolysis of IMB-1018972 to IMB-1028814 and subsequent systemic bioavailability of IMB-1028814 was relatively rapid with median tmax ranging between 0.5 hours and 5 hours postdose for IMB-1028814, and between 1.5 hours and 8 hours postdose for trimetazidine. Median tmax did not increase with increasing IMB-1018972 dose
The predefined stopping criterion for IMB-1028814 plasma exposure of 417,733 and 652,849 ng·h/mL for males and females, respectively, was not reached by any of the subjects during the SAD part or MAD part.
Following single oral IMB-1018972 doses in the range of 50 to 400 mg under fasted conditions, systemic exposure to IMB-1028814 and trimetazidine was dose proportional for Cₘₐₓ, AUC₀₋ₜ, and AUC_{0-inf}.
No evidence for an effect of food was observed on the IMB-1028814 exposure parameters AUC₀₋ₜ and AUC_{0-inf} following administration of a single dose of 150 mg IMB-1018972. However, Cₘₐₓ was approximately 36% lower following administration of a single dose of 150 mg IMB-1018972 under fed conditions relative to administration under fasted conditions.
No evidence for an effect of food was observed on the trimetazidine exposure parameters Cₘₐₓ, AUC₀₋ₜ, and AUC_{0-inf} following administration of a single dose of 150 mg IMB-1018972.
When combining the single and multiple IMB-1018972 dose results under fasted and fed conditions, the geometric mean t1/2 ranged between 2.5 hours and 4.5 hours for IMB-1028814, and between 6.5 hours and 9.5 hours for trimetazidine. Geometric mean t_{1/2} did not increase with increasing IMB-1018972 dose.
Within 48 hours following administration of a single oral dose of IMB-1018972 over the range of 50 mg to 400 mg, on average between 3.99% and 5.74% of the dose was excreted in urine as IMB-1028814, and on average between 23.11% and 32.55% of the dose was excreted as trimetazidine.
Within 48 hours following administration of a single oral dose of 35 mg trimetazidine, on average 54.47% of the dose was excreted in urine as trimetazidine.
Following 14 days of twice daily dosing with 150 mg and 50 mg IMB-1018972 under fed conditions, no relevant accumulation was observed of IMB-1028814 (Rac of 1.18 and 1.10 for 150 mg and 50 mg, respectively) and accumulation of trimetazidine was modest (Rac of 1.63 and 1.89 for 150 mg and 50 mg, respectively).

### Overall

In view of the positive risk/benefit profile and the observed PK characteristics of the IMB-1018972 metabolites IMB-1028814 and trimetazidine in this single-dose and multiple-dose FIH study, further clinical development of IMB-1018972 is warranted.

### Detailed Study Design

### Introduction

IMB-1018972 is an orally administered small molecule that is being developed as a treatment for ischemic cardiovascular disease and the associated abnormal cellular energetics. Potential indications include angina pectoris, heart failure, and peripheral vascular disease. IMB-1018972 is a new chemical entity (NCE) of the drug class partial fatty acid oxidation (pFOX) inhibitors that acts to preserve or enhance energy metabolism in cells exposed to hypoxia or ischemia. Other pFOX inhibitors include ranolazine (Ranexa), perhexiline, and trimetazidine. Glucose oxidation is a more efficient producer of adenosine triphosphate per oxygen molecule consumed compared to fatty acid oxidation.

IMB-1018972 undergoes hydrolysis after administration, and the hydrolysis products are nicotinic acid (also known as niacin or vitamin B3) and an inhibitor of 3-ketoacyl CoA thiolase (3-KAT) named IMB-1028814. In addition to IMB-1018972, IMB-1028814 has been studied and characterized extensively in nonclinical studies. IMB-1028814 undergoes further metabolism and 1 metabolite is trimetazidine, a drug marketed in Europe since 1987 for the treatment of angina pectoris.

The primary mechanism of action of IMB-1028814 is thought to be competitive inhibition of 3-KAT that results in the shift of substrate utilization in the myocardium from fatty acid oxidation to glucose oxidation. The delivery of nicotinic acid may serve to additionally enhance cellular energetics.

The nonclinical pharmacology and toxicology data collected at the time the CSP was finalized supported conducting clinical studies that administer IMB-1018972 for up to 4 weeks to assess its safety, tolerability, PK, and pharmacodynamics in humans.

Trimetazidine administered in this study is a drug marketed in Europe since 1978 for the treatment of angina pectoris.

### Study Rationale

No clinical studies with IMB-1018972 had been performed prior to the study described in this CSR. Therefore, this first-in-human study (FIH), with single-dose and multiple-dose escalation designs (single ascending dose [SAD] part and multiple ascending dose [MAD] part) and a single-dose food effect (FE) study was conducted to assess the safety, tolerability, and PK of IMB-1018972 as an immediate-release (IR) formulation following administration of single and multiple ascending doses.

During the study, a group was added to the SAD part testing a single 35-mg modified-release (MR) dose of trimetazidine (Vastarel). The primary rationale for adding this group was to study the PK profile of commercially available trimetazidine with the same analytical assays utilized in the current study, which would enable a direct comparison of the PK profiles of trimetazidine generated from Vastarel and that generated from the metabolism of IMB-1028814. The analytical assays include detection of trimetazidine in blood and urine, which is the primary route of elimination. These data, together with the data generated from the MAD part would help the Sponsor select doses for further investigation in the Phase 2 proof-of-concept study in patients with refractory angina.

### Study Objectives

### Primary

To assess the safety and tolerability of single and multiple ascending oral doses of IMB-1018972, single oral doses of trimetazidine.

### Secondary

To assess the PK profile of single and multiple ascending oral doses of IMB-1018972, single oral doses of trimetazidine.
To assess the effect of food on the absorption and the PK profile of IMB-1018972 following a single oral dose of IMB-1018972 in healthy subjects.
To evaluate the effect of food on the safety and tolerability of IMB-1018972 following a single oral dose of IMB-1018972 in healthy subjects

### Investigational Plan

### Overall Study Design and Plan

### Type of Study

This was a double-blind, randomized, placebo-controlled study, consisting of a SAD part with integrated FE arm, a MAD part to assess the safety, tolerability, and PK of ascending single and multiple oral doses of IMB-1018972 (IR formulation in the SAD and MAD parts), and single oral doses of a MR formulation of trimetazidine. The study started with the SAD part.

### SAD Part (and Integrated FE Arm)

In the SAD part, 5 groups of 8 healthy subjects (6 subjects on active drug and 2 on placebo in Groups A1, A2, A3, and A4, and 8 subjects on active drug in Group A5) were included. In Groups A1, A2, A3, and A4, subjects received a single oral dose of an IR formulation of IMB-1018972 or placebo under fasted conditions (an overnight fast of at least 10 hours). In Group A5, all subjects received a single oral dose of a MR formulation of trimetazidine under fasted conditions (an overnight fast of at least 10 hours). Each subject participated in only 1 group during the study

Subjects assigned to Group A4 also participated in the FE arm and received the same single dose of IMB-1018972 or placebo under fed conditions (Food and Drug Administration [FDA]-defined high-fat breakfast after an overnight fast of at least 10 hours) in a second period at least 1 week after drug administration under fasted conditions in the SAD part.

In this first-in-human (FIH) study, the subjects participating at the lowest dose level, subjects of Group A1, were dosed according to a sentinel dosing design to ensure optimal safety. This means that initially, 2 subjects were dosed: 1 subject with IMB-1018972 and 1 subject with placebo. Since the safety and tolerability results of the first 24 hours following dosing for the initial 2 subjects were acceptable to the Investigator, the other 6 subjects (5 active drug and 1 placebo) of the lowest dose level were also dosed. Depending on emerging safety data, it could have been decided to implement this sentinel dosing design for other groups as well; however, this was not done.

The SAD part consisted of:
An eligibility screening period of up to 35 days
One study period involving administration of a single dose of IMB-1018972 or placebo (or trimetazidine in Group A5); this was 2 periods for subjects of Group A4 also participating in the FE arm
Safety assessments and blood sampling for PK purposes from predose up to 48 hours after drug administration
Discharge at 48 hours after study drug administration (in each period for subjects of Group A4 also participating in the FE arm)·
A follow-up visit 7 to 14 days after the last PK blood sample; this was 7 to 14 days after the last PK blood sample in the second period for subjects of Group A4 also participating in the FE arm

### MAD Part

In the MAD part, 2 groups of 12 healthy subjects (9 subjects on active drug and 3 on placebo in each group) were included. Subjects received multiple oral doses of an IR formulation of IMB-1018972 or placebo once q12 h for 14 consecutive days. Each subject participated in only 1 group during the study.

Study drug administration was under fed conditions as determined based on the results of Group A4 in the FE arm.

The MAD part consisted of:
An eligibility screening period of up to 35 days
One study period involving administration of multiple doses of IMB-1018972 or placebo for 14 consecutive days
Safety assessments and blood sampling for PK purposes from predose up to 48 hours after the last study drug administration.
Discharge at 48 hours after the last study drug administration
A follow-up visit 7 to 14 days after the last PK blood sample

### Screen Period

Subjects reported to the medical screening facility for the eligibility screening within 5 weeks prior to (the first) study drug administration.

Subjects signed the study-specific ICF prior to any study-specific screening procedures being performed. The written informed consent was obtained for all subjects, regardless of their eligibility for the study. The signed ICFs were retained and archived at PRA and a copy was provided to the subject.

### Treatment Period

Subjects were in the clinic for 1 treatment period (2 treatment periods for subjects of Group A4 also participating in the FE arm). The subjects were admitted to the clinical research center in the afternoon of Day −1. Day 1 was the day of (the first) drug administration.

Subjects of the SAD part were discharged on Day 3 (48 hours after study drug administration) after completion of the assessments; discharge was on Day 3 of each period for subjects of Group A4 also participating in the FE arm. Subjects of the MAD part were discharged on Day 16 (48 hours after the last study drug administration on Day 14) after completion of the assessments.

### Follow-Up

For the SAD part, the follow-up assessments were performed 7 to 14 days after the last PK blood sample (between Day 10 and Day 17). For the FE arm, the follow-up assessments were performed 7 to 14 days after the last PK blood sample in the second period (between Day 10 and Day 17). For the MAD part, the follow-up assessments were performed 7 to 14 days after the last PK blood sample (between Day 23 and Day 30).

### Discussion of Study Design

Dose Escalation within a Study Part

An escalating-dose study design was chosen for the SAD and MAD parts to allow careful increase of the IMB-1018972 dose after assessment of the available safety, tolerability, and PK results of the preceding group.

A dose-escalation meeting was held between the Investigator and the Sponsor. Further, a dose-escalation report (DER) was provided by the Investigator to the IEC following completion of each dose level. Escalation to the next higher dose only proceeded when none of the stopping criteria had been reached and if the available safety, tolerability, and PK results of the preceding group were acceptable to the Investigator and the Sponsor and after a statement of no objection of the DER from the IEC. The safety, tolerability, and PK results had to be available up to 48 hours postdose for the SAD part and up to 48 hours after the final morning dose on Day 14 for the MAD part. In addition, these results had to be available from at least 5 dosed subjects of the preceding group in the SAD part and at least 8 dosed subjects of the preceding group in the MAD part

The planned dose levels to be administered could be changed based on the safety, tolerability, and plasma PK results of the previous group(s).

Dose levels in the MAD part could not exceed dose levels that were well tolerated in the SAD part.

The increase from one dose level to the next dose level could not be more than 3-fold.

Although this was an ascending dose study, if safety or tolerability issues were experienced, a lower dose could be administered in the next groups. Also, the same dose could be tested in 2 groups or an intermediate dose could by tested to gain more information on safety, tolerability, and/or PK.

### Stopping Rules for Dose Escalation

Dosing within a group and dose escalation to a next group was halted at any time if 1 of the following circumstances occurred:
A drug-related serious adverse reaction (ie, a serious adverse event [SAE] considered at least possibly related to the study drug administration) in 1 subject.
Drug-related severe adverse reactions (ie, severe adverse events [AEs] considered at least possibly related to the study drug administration) in 2 subjects in the same group, or in 1 subject in the sentinel group of a group.
Other findings that, at the discretion of the Investigator and/or Sponsor's Medical Monitor, indicated that further dosing had to be stopped.

When stopping rules for a group were met, the randomization code for subjects meeting the stopping rules was to be unblinded. If after unblinding it was concluded that subjects on active medication met the stopping rules, dosing in the group was to be stopped, and no further dose escalation was to be performed. If a subsequent integrated analysis of available data led to the conclusion that further careful escalation was warranted, a substantial amendment was needed before continuation of the study. Dose escalation in a study part (SAD, MAD) was permanently stopped if:
Blinded PK data indicated that after dose escalation it was anticipated that individual subjects would exceed the predefined maximum exposure level of AUC0-8×2 for IMB-1028814 of 417,733 and 652,849 ng·h/mL for males and females, respectively

### Sentinel Dosing

IMB-1018972 is in the early stage of clinical development, with the SAD part of the study being the first time the compound was administered to man. In this FIH study, the subjects participating at the lowest dose level of the SAD part, subjects of Group A1, were dosed according to a sentinel dosing design to ensure optimal safety. This means that initially, 2 subjects were dosed. One of these subjects received the active medication IMB-1018972, and the other subject received placebo. The subjects were closely observed by the Investigator for the first 24 hours following drug administration. The general tolerability of the study drug was monitored during this time, and the electrocardiogram (ECG) and vital signs recordings were reviewed. Any reported AEs were also considered in the Investigator's evaluation. If the safety and tolerability results of the first 24 hours following dosing for the initial 2 subjects were acceptable to the Investigator, the other subjects of the lowest dose level could be dosed in a placebo-controlled randomized manner (5 active and 1 placebo). Depending on emerging safety data, it could have been decided to implement this sentinel dosing design for other SAD groups as well (except for the second period of Group A4 in the FE arm and except for Group A5; all subjects of these 2 groups could be dosed on the same day).

### Effect of Food

Subjects from Group A4 of the SAD part were assigned to the integrated FE arm. After administration of the drug to fasting subjects in the SAD part, the FE arm used the same subjects and experimental procedures. An exception was that subjects consumed an FDA-defined high-fat breakfast prior to dosing to evaluate the possible effect of food on the PK of IMB-1018972. This allowed for a within-subject comparison of the PK of IMB-1018972 in plasma and tolerability after administration in fasted and fed conditions.

### Continuation to the MAD Part of the Study

The MAD part could start after the results from the FE arm were available. The first group of the MAD part could start when a DER, summarizing safety and available PK data of previous SAD groups, concluded that a single dose with an exposure at/above the expected steady-state exposure in the first MAD group was well tolerated.

In the MAD part, subjects received twice daily dosing, which was the anticipated clinical dosing regimen considering the anticipated short human half-life and absence of prolonged duration of action. Doses were given q12 h. In the MAD part, dosing continued for 14 days, which was anticipated to result in steady state of exposure.

The highest multiple-dose group planned could not exceed the highest planned single dose of 1600 mg/day or the highest tolerated dose in the SAD part. This was predicted to sufficiently cover doses in future dose-finding studies in patients.

### Other

The planned confinement period, day of discharge, and follow-up period could be adapted depending on emerging study results. Also, the timing, type, and number of safety and PK assessments could be changed during the study.

The purpose of including placebo-treated subjects in each group (except Group A5 in which no placebo was administered) was to assist the medical assessment of whether or not any abnormalities observed were due to the study drug or to study procedures, and not for a formal statistical comparison between active and placebo subjects.

There was no indication from in vitro studies (cytochrome P450 [CYP]3A4/GT1A1/CYP2C19/CYP2C9) for interaction with oral contraceptives. Women of childbearing potential who were using adequate contraception were included in the present study, in order to make the outcome of this FIH study relevant for the female target patient population.

The use of healthy subjects as opposed to patients allowed a clearer interpretation of the study results, as there were no confounding factors resulting from changes in disease state and/or concomitant medications.

The study was performed in different groups of subjects since the number of doses to be tested, and all assessments associated with these sessions, were regarded as too extensive to be performed in a single group of subjects participating repeatedly.

The Investigator took all the usual precautions necessary for studies at an early stage in the development of a new drug.

### Selection of Study Population

The overall study population consisted of 88 subjects.

In the SAD part (and integrated FE part), a total of 40 healthy male or female subjects were included. Eight subjects from Group A4 in the SAD part participated in the FE arm. From Group A4 onwards, all efforts were made to have a ratio of 50:50 for male and female subjects per group, but at minimum at least 3 subjects of each gender were dosed per group.

In the MAD part, a total of 24 healthy male or female subjects were included. For each group, all efforts were made to have a ratio of 50:50 for male and female subjects, but at minimum at least 4 subjects of each gender were dosed per group.

All efforts were made to have a ratio of 50:50 for male and female subjects, but at minimum at least 4 subjects of each gender were dosed in each part.

### Inclusion Criteria

Subjects were eligible for inclusion in the study if they met all the following inclusion criteria:
1. Gender: male or female.
2. Age: 18 years to 65 years, inclusive, at screening.
3. Body mass index (BMI): 18.0 kg/m2 to 32.0 kg/m2, inclusive.
4. Status: healthy subjects.
5. At screening, females could be of childbearing potential (but not pregnant or lactating), or of nonchildbearing potential (either surgically sterilized or physiologically incapable of becoming pregnant, or at least 1 year postmenopausal [amenorrhea duration of 12 consecutive months]); nonpregnancy was confirmed for all females by a serum pregnancy test conducted at screening and each admission.
6. Female subjects of childbearing potential who had a fertile male sexual partner had to agree to use adequate contraception from screening until 90 days after the follow-up visit. Adequate contraception was defined as using hormonal contraceptives or an intrauterine device combined with at least 1 of the following forms of contraception: a diaphragm, a cervical cap, or a condom. Total abstinence, in accordance with the lifestyle of the subject, was also acceptable.
7. Male subjects, if not surgically sterilized, had to agree to use adequate contraception and not donate sperm from (first) admission to the clinical research center until 90 days after the follow-up visit. Adequate contraception for the male subject (and his female partner) was defined as using hormonal contraceptives or an intrauterine device combined with at least 1 of the following forms of contraception: a diaphragm, a cervical cap, or a condom. Total abstinence, in accordance with the lifestyle of the subject, was also acceptable.
8. All prescribed medication had to be stopped at least 30 days prior to (first) admission to the clinical research center. An exception was made for hormonal contraceptives, which could be used throughout the study.
9. All over-the-counter medication, vitamin preparations and other food supplements, or herbal medications (eg, St. John's Wort) had to be stopped at least 14 days prior to (first) admission to the clinical research center. An exception was made for paracetamol, which was allowed up to admission to the clinical research center.
10. Willingness to abstain from alcohol, methylxanthine-containing beverages or food (coffee, tea, cola, chocolate, energy drinks), grapefruit (juice), and tobacco products from 48 hours prior to (each) admission to the clinical research center.
11 Good physical and mental health on the basis of medical history, physical examination, clinical laboratory, and vital signs, as judged by the Investigator.
12. Had no clinically significant abnormal 12-lead ECG (incomplete right bundle branch block could be accepted) at screening: PR-interval<210 ms, QRS-duration<120 ms, and QTc-interval (Fridericia's)≤450 msec for males and females.
13 Willing and able to sign the ICF.

### Exclusion Criteria

Subjects were excluded from participation if any of the following exclusion criteria applied:
1. Previous participation in the current study.
2. Employee of PRA or the Sponsor.
3. History of relevant drug and/or food allergies.
4. Using tobacco products within 3 months prior to (the first) drug administration.
5. History of alcohol abuse or drug addiction (including soft drugs like cannabis products).
6. Positive drug and alcohol screen (opiates, methadone, cocaine, amphetamines [including ecstasy], cannabinoids, barbiturates, benzodiazepines, tricyclic antidepressants, and alcohol) at screening and (each) admission to the clinical research center.
7. Average intake of more than 24 units of alcohol per week (1 unit of alcohol equals approximately 250 mL of beer, 100 mL of wine, or 35 mL of spirits).
8. Positive screen for hepatitis B surface antigen (HBsAg), anti-hepatitis C virus (HCV) antibodies, or anti-HIV 1 and 2 antibodies.
9. Participation in a drug study within 60 days prior to (the first) drug administration in the current study. Participation in more than 4 other drug studies in the 12 months prior to (the first) drug administration in the current study.
10. Donation or loss of more than 100 mL of blood within 60 days prior to (the first) drug administration. Donation or loss of more than 1.5 liters of blood (for male subjects)/more than 1.0 liters of blood (for female subjects) in the 10 months prior to (the first) drug administration in the current study.
11. Significant and/or acute illness within 5 days prior to (the first) drug administration that could impact safety assessments, in the opinion of the Investigator.
12. Unsuitable veins for infusion or blood sampling.
13. For FE Group A4 and the single-dose MR part only: Unwillingness to consume the FDA breakfast.

Please note that subjects were to refrain from consumption of any foods containing poppy seeds within 48 hours (2 days) prior to screening to the clinical research center to avoid false positive drug screen results. In addition, they were to refrain from strenuous exercise within 96 hours (4 days) prior to screening as this could result in abnormal clinical laboratory values.

Removal of Subject from Assessment

Participation in the study was strictly voluntary. A subject had the right to withdraw from the study at any time for any reason.

The Investigator had the right to terminate participation of a subject for any of the following reasons: difficulties in obtaining blood samples, violation of the protocol, severe AEs or SAEs, or for any other reason relating to the subject's safety or the integrity of the study data.

If a subject was withdrawn from the study, the Sponsor was to be informed immediately. If there was a medical reason for withdrawal, the subject remained under the supervision of the Investigator until satisfactory health had returned.

Subjects who dropped out or withdrew for any reason without completing all screening evaluations successfully, were considered screening failures.

A subject who was withdrawn or voluntarily withdrew from the study for any reason, whether related to the study drug or not, after having received a subject number, was considered an early-termination subject. If a subject was withdrawn for a reason related to the study drug, according to the judgment of the Investigator, the early-termination subject was not replaced. If a subject did not complete the study for a reason not related to the study drug, the early-termination subject could be replaced after mutual agreement between the Sponsor and PRA.

The decision regarding the replacement of subjects was documented.

PRA made every effort to ensure that early-termination subjects who had received study drug completed the safety follow-up assessments.

### Stopping Rules for Individual Subjects

Dosing of a subject was stopped at any time during the study if any of the following circumstances occurred:
A serious adverse reaction (ie, an SAE considered at least possibly related to the study drug administration).
An overall pattern of clinically significant changes in any safety parameter (eg, moderate or severe AEs in >1 subject) that could appear to be minor in terms of an individual event but, in the opinion of the Sponsor or Investigator, collectively represented a safety concern.
Other findings that, at the discretion of the Investigator and/or Sponsor's Medical Monitor, indicated that further dosing should be stopped.

### Treatments

### SAD Part (and Integrated FE Arm)

The following treatments were administered under fasted conditions according to the randomization code:
Group A1 single oral dose of 50 mg IR formulation of IMB-1018972 (n=6) or matching placebo (n=2) on Day 1
Group A2 single oral dose of 150 mg IR formulation of IMB-1018972 (n=6) or matching placebo (n=2) on Day 1
Group A3: single oral dose of 400 mg IR formulation of IMB-1018972 (n=6) or matching placebo (n=2) on Day 1
Group A4: single oral dose of 150 mg IR formulation of IMB-1018972 (n=6) or matching placebo (n=2) on Day 1 (FE group)
Group A5: single oral dose of 35 mg MR formulation of trimetazidine (Vastarel; n=8) on Day 1

The following treatment was administered in the FE arm under fed conditions (FDA-defined high-fat breakfast) according to the randomization code
Group A4: single oral dose of 150 mg IR formulation of IMB-1018972 (n=5) or matching placebo (n=2) on Day 1 (same dose as in SAD part)

Up to 2 additional SAD groups could be included to evaluate a lower, intermediate, or repeat dose level(s), or, provided that dose-escalation termination criteria had not been met, a higher dose level.

### MAD Part

The following treatments were administered according to the randomization code under fed conditions as determined based on the results of Group A4 in the FE arm. The doses were selected based upon the safety, tolerability, and PK data from the SAD part:
Group B1: multiple oral doses of an IR formulation of 150 mg IMB-1018972 (n=9) or matching placebo (n=3) twice daily (q12 h) for 14 days; on Day 14 only a single morning dose was administered
Group B2: multiple oral doses of an IR formulation of 50 mg IMB-1018972 (n=9) or matching placebo (n=3) q12 h for 14 days; on Day 14 only a single morning dose was administered

Up to 2 additional MAD groups could be included to evaluate a lower, intermediate, or repeat dose level(s), or, provided that dose-escalation termination criteria had not been met, a higher dose level.

### Identity of Investigational Products

### Active Medication

Drug product: IMB-1018972
Activity: Fatty acid oxidation inhibitor
In development for: Ischemic cardiovascular disease
Strength: 25 mg, 100 mg, and 200 mg IR formulations (based on free base)
Dosage form: Oral IR capsule(s) to be used in the SAD and MAD parts
Manufacturer: Pharmacy at PRA
Batch number: 2479-1810-00441 (drug substance)

### IMB-1018972 Placebo (Visually Matching Active Medication)

Active substance: Not applicable
Activity: Not applicable
Strength: Not applicable
Dosage form: Oral capsule(s)
Manufacturer: Pharmacy at PRA
Batch number: Not applicable

### Active Medication

Drug product: Vastarel MR (trimetazidine dihydrochloride)
Activity: Fatty acid oxidation inhibitor
In development for: Angina pectoris
Strength: 35 mg
Dosage form: Oral MR tablet
Manufacturer: Servier Research & Pharmaceuticals (Pakistan) (Pvt.) Ltd.
Batch number: 273782 (drug product)

The study drug was stored in the pharmacy at PRA in a locked facility under the required storage conditions with continuous monitoring. The study drug was dispensed by the pharmacist to the Investigator or authorized designee.

The total number of IMB-1018972 capsules given per dose level in the SAD part (and integrated FE arm) and MAD part is given in Table 2. The number of placebo capsules that was administered to a placebo subject in a specific group was the same as the number of IMB-1018972 capsules that was given to an IMB-1018972 subject in that group.

TABLE 2
Number of IMB-1018972 Capsules Given per Dose Level
in the SAD Part (and Integrated FE Arm) and MAD Part

| | Number of | Number of | Number of | |
|---|---|---|---|---|
| | 25-mg | 100-mg | 200-mg | |
| | (free base) | (free base) | (free base) | Total number of |
| IMB-1018972 | IMB-1018972 | IMB-1018972 | IMB-1018972 | IMB-1018972 |
| dose level | capsules | capsules | capsules | capsules |
| 50 | 2 | 0 | 0 | 2 |
| 150 | 2 | 1 | 0 | 3 |
| 400 | 0 | 0 | 2 | 2 |

### Method of Assignment Subjects to Treatment Groups

After obtaining informed consent, subjects were screened according to the inclusion and exclusion criteria. Subjects who met all eligibility criteria received a subject number upon inclusion in the study. They received the subject number just prior to dosing according to the randomization code generated by the Biostatistics Department of PRA. The subject number ensured identification throughout the study.

Subject numbers were 101 to 140 for the SAD part, 201 to 224 for the MAD part. Any additional subjects to be included in the SAD part were to be numbered starting from subject number 141 and any additional subjects in the MAD part were to be numbered starting from subject number 225.

Any replacement subject was to receive the number of the subject to be replaced, increased by 200, and was to be administered the same treatment(s). Subjects were assigned to a study part and group based on their availability. Treatments within a group were assigned according to the randomization code generated by the Biostatistics Department of PRA.

In each SAD group, except for Group A5, 6 subjects were randomly assigned to receive IMB-1018972 and 2 subjects were randomly assigned to receive placebo. In Group A5, all 8 subjects received trimetazidine. In each MAD group, 9 subjects were randomly assigned to receive IMB-1018972 and 3 subjects were randomly assigned to receive placebo.

For the 2 sentinel subjects in Group A1 of the SAD part, randomization ensured that 1 subject received IMB-1018972 and the other subject received placebo. For the remaining 6 subjects of Group A1, randomization ensured that 5 received IMB-1018972 and 1 received placebo. Depending on emerging safety data, it could be decided to implement this sentinel dosing design for other SAD groups as well (except for the second period of Group A4 in the FE arm and except for Group A5; all subjects of these 2 groups could be dosed on the same day).

Subjects who dropped out or withdrew for any reason without completing all screening evaluations successfully were considered screening failures. Such subjects, and also subjects who were eligible for inclusion in the study but did not receive the study drug, received no subject number, and only applicable data were entered in the eCRFs.

### Selection of Doses in the Study

Based on the nonclinical toxicology data, it was considered that subjects in this clinical study were not at unreasonable risk of adverse effects. Based on the 28-day dog no observed adverse effect level (NOAEL) of 200 mg/kg/day (oral), the calculated human equivalent dose (HED) is 108 mg/kg/day. For a 60-kg individual, the NOAEL dose would be 6480 mg. With a 10-fold safety factor applied, this would allow for a maximum recommended starting dose (MRSD) of 648 mg/day.7,8 The planned starting dose in the current Phase 1 study was 50 mg, equivalent to 0.83 mg/kg/day for a 60-kg subject. This starting dose is less than 10% of the MRSD determined from the dog NOAEL and less than 1% of the dog NOAEL.

The maximum planned dose in this study of 1600 mg in healthy volunteers was 25% of the HED NOAEL dose of 6480 mg and only 2.5 fold higher than the MRSD. The conservative dosing margin was expected to cover potential supratherapeutic exposures, for instance in patients with renal or hepatic impairment, or in case of potential drug interactions with IMB-1018972. This risk for healthy volunteers at these exposure levels was determined to be acceptable based on the absence of irreversible or significant toxicities without sentinel safety biomarkers.

The relevant animal study was the 28-day dog study where the NOAEL for IMB-1018972 was 200 mg/kg/day. The AUC_{0-8×2} for IMB-1028814 on Day 26 at this dose was 417,733 and 652,849 ng·h/mL for males and females, respectively. The AUC_{0-8×2} for trimetazidine on Day 26 at this dose was 15,042, and 13,834 ng·h/mL for males and females, respectively.

A cohort was added by the Sponsor that was testing a single 35 mg MR dose of trimetazidine (Vastarel). This dose was selected as it is the most commonly used dose of trimetazidine in treating angina and it was therefore known that it has an efficacious PK profile.

### Timing of Doses in the Study

The study drug was administered with 240 mL of tap water to the subject in the upright position. If needed, an additional volume of water was allowed to consume the capsules/tablets comfortably; this additional volume was documented in the eCRF. The dose was given between 08:00 h and 11:00 h, and between 20:00 h and 23:00 h for the afternoon/evening dose. Dosing for each individual subject was at around the same time (+15 min) on each dosing day. The study drug was not chewed.

Administration of the study drug was supervised by the Investigator or authorized designee. After drug administration, a mouth and hand inspection took place.

### Dosing Under Fasted Conditions

SAD Part 9 and Integrated FE Arm) Before dosing, subjects fasted overnight for at least 10 hours following a light supper on the evening before. Following dosing, subjects fasted for 4 hours until lunch. During fasting, fluids other than water were not allowed; however, water was not allowed from 2 hours predose until 1 hour postdose (apart from the water taken with the dose).

Subjects of Group A4, also participating in the FE arm, were not allowed to lie down for 4 hours after dosing, except when required for assessments that needed to be performed.

### Dosing Under Fed Conditions

### FE Arm

Before dosing, subjects fasted overnight for at least 10 hours following a snack on the evening before. Then, subjects received an FDA-defined high-fat breakfast that had to be consumed within 20 minutes. The entire breakfast had to be consumed by the subjects. Dosing occurred at 30 minutes after the start of breakfast. Following dosing, subjects fasted for 4 hours until lunch. During fasting, fluids other than water were not allowed.

Subjects of Group A4 also participating in the FE arm were not allowed to lie down for 4 hours after dosing, except when required for assessments that needed to be performed.

### MAD Arm

### Morning Dose

Before each morning dose, subjects fasted overnight for at least 10 hours following a snack on the evening before. On Days 1 and 14, subjects received a standardized breakfast that had to be consumed within 20 minutes. Dosing occurred at 30 minutes after the start of breakfast. Following dosing, subjects fasted for 4 hours until lunch. During fasting, fluids other than water were not allowed. On Days 2 to 13, breakfast was not standardized and was given within maximally 1 hour before dosing and consumed before dosing. No fasting after dosing was applicable on these days.

### Evening Dose

On all dosing days, an evening snack was given within maximally 1 hour before dosing and consumed before dosing.

### Meals During the Study

A fasting period of at least 4 hours was required before obtaining clinical laboratory samples at all time points.

When not fasting, meals and snacks (such as decaffeinated coffee, herbal tea, fruit, and biscuits) were provided according to PRA standard operating procedures (SOPs). A light supper was provided on the evening before those days where fasting was required until lunch time (fasted conditions); a snack was provided on the evening before those days where fasting was required until the FDA-defined high-fat breakfast or breakfast (fed conditions).

For the second period of Group A4 in the FE arm, the FDA-defined high-fat breakfast of 918 kcal consisted of:
2 fried eggs (in 15 g butter/margarine) (approximately 100 g)
1 portion of bacon (40 g) (or brie 60+ for vegetarians)
1 portion of fried potatoes (115 g)
2 slices of (toasted) (wheat) bread (approximately 70 g) with 15 g margarine
1 glass of whole milk (240 mL)
   The total of 918 kcal (vegetarian version 915 kcal) could be broken down as follows:
39 g protein=156 kcal
59 g fat=527 kcal
59 g carbohydrates=235 kcal

### Blinding

In each group of the SAD part, except for Group A5, 6 subjects received IMB-1018972 and 2 subjects received placebo according to the randomization code. In Group A5, all 8 subjects received trimetazidine. In each group of the MAD part, 9 subjects received IMB-1018972, and 3 subjects received placebo according to the randomization code. The following controls were employed to maintain the double-blind status of the study:
The oral capsules containing active drug or placebo were indistinguishable in
appearance and taste.
The randomization code was provided to the pharmacist at PRA for dispensing
purposes and kept in the pharmacy, accessible to the pharmacist and the pharmacy
assistant only.

Individual code break envelopes were provided for all subjects by PRA. Each sealed envelope containing the randomization code was kept in a medication storage room that was locked with restricted access. To manage the subject's condition in case of a medical emergency, the Investigator was allowed to break the code to know whether a subject received IMB-1018972 or placebo. If opened, the name of the person who opened it, the date and time of opening, and the reason for opening were to be written on the envelope. The Sponsor was to be informed in case of unblinding.

The Bioanalytical Laboratory of PRA where the PK samples were analyzed was provided a copy of the randomization code by the pharmacy since only samples of subjects who had received the active drug IMB-1018972 were to be analyzed.

### Previous and Concomitant Therapy and Other Restrictions During the Study

The use of all prescribed medication was not allowed from (first) admission to the clinical research center until follow-up. An exception was made for hormonal contraceptives, which were allowed throughout the study. The use of all over-the-counter medication, vitamin preparations and other food supplements, or herbal medications (eg, St. John's Wort) was not allowed from (first) admission to the clinical research center until follow-up. An exception was made for paracetamol: from (first) admission onwards, the Investigator could permit a limited amount of paracetamol for the treatment of headache or any other pain. Other medication to treat AEs could only be prescribed if deemed necessary by the Investigator. If medication was used, the name of the drug, the dose, and dosage regimen were recorded in the eCRF.

The use of alcohol, methylxanthine-containing beverages or food (coffee, tea, cola, chocolate, energy drinks), grapefruit (juice), and tobacco products was not allowed during the stay in the clinical research center.

Strenuous exercise was not allowed within 96 hours (4 days) prior to (each) admission and during the stay(s) in the clinical research center.

Subjects were not allowed to consume any foods containing poppy seeds within 48 hours (2 days) prior to (each) admission to the clinical research center as this could cause a false positive drug screen result.

Female subjects of childbearing potential, with a fertile male sexual partner, were required to use adequate contraception (see description below) from screening until 90 days after the follow-up visit.

Male subjects, if not surgically sterilized, were required to use adequate contraception (see description below) and not donate sperm from (first) admission to the clinical research center until 90 days after the follow-up visit.

Adequate contraception was defined as using hormonal contraceptives or an intrauterine device combined with at least 1 of the following forms of contraception: a diaphragm, a cervical cap, or a condom. Total abstinence, in accordance with the lifestyle of the subject, was also acceptable.

Subjects were not allowed to donate blood during the study until the follow-up visit (other than the blood sampling planned for this study).

### Treatment Compliance

Study drug was administered in the clinical research center. To ensure treatment compliance, administration of the study drug was supervised by the Investigator or authorized designee. Compliance was further confirmed by bioanalytical assessment of IMB-1018972, IMB-1028814, and trimetazidine in plasma and urine samples.

The exact times of study drug administration and the number of units administered were recorded in the eCRF. Drug accountability procedures as specified in the CSP were followed.

### Safety and Pharmacokinetic Measurements and Variables

The present study was performed to assess safety, tolerability, and PK following single and multiple doses of single and multiple oral doses of IMB-1018972, single oral doses of trimetazidine. This study did not comprise efficacy or pharmacodynamic assessments.

### Adverse Events

AEs were recorded from (first) admission until completion of the follow-up visit. Any clinically significant observations in results of clinical laboratory, 12-lead ECGs, vital signs, or physical examinations were recorded as AEs.

A treatment-emergent AE (TEAE) was defined as any event not present prior to (the first) administration of the study drug or any event already present that worsened in either severity or frequency following exposure to the study drug.

An AE that occurred prior to (the first) administration of the study drug was considered a pretreatment AE.

At several time points before and after drug administration, subjects were asked nonleading questions to determine the occurrence of AEs. Subjects were asked in general terms about any AEs at regular intervals during the study. In addition, all AEs reported spontaneously during the course of the study were recorded. Details included description of the event, date and time of onset, date and time of end, total duration, severity, relationship to study drug, intervention, seriousness, and outcome. All answers were interpreted by the Investigator and were recorded in the eCRF. All AEs were classified according to the Medical Dictionary for Regulatory Activities (MedDRA; Version 22.0) for AEs.

The severity of the AEs was rated as mild, moderate, or severe; the relationship between the AEs and the study drug was indicated as none, unlikely, possibly, likely, or definitely. Adverse events assessed as possibly, likely, or definitely were considered related to the study drug; AEs assessed as none or unlikely were considered not related to the study drug.

Concomitant medication or other therapy required in case of any AEs was recorded. Concomitant medications were classified according to the World Health Organization Drug Dictionary (Version 22.0).

All AEs were followed up until their resolution or stabilization.

### Clinical Laboratory

Blood and urine samples for clinical laboratory assessments were collected according to PRA SOPs.

The following parameters were measured:
Clinical chemistry (serum quantitatively): total bilirubin, alkaline phosphatase, gamma glutamyl transferase, aspartate aminotransferase (AST), alanine aminotransferase (ALT), lactate dehydrogenase, creatinine, urea, total protein, glucose, inorganic phosphate, sodium, potassium,
calcium, and chloride
Hematology (blood quantitatively): leukocytes, erythrocytes, hemoglobin, hematocrit, thrombocytes, partial automated differentiation (lymphocytes, monocytes, eosinophils, basophils, and neutrophils), mean corpuscular volume, mean corpuscular hemoglobin, and mean corpuscular hemoglobin concentration
Coagulation (blood quantitatively): prothrombin time (reported in seconds and as international normalized ratio), activated partial thromboplastin time, and fibrinogen
Urinalysis (urine qualitatively): hemoglobin, urobilinogen, ketones, glucose, and protein
Serology: HBsAg, anti-HCV, and anti-HIV 1 and 2
Drug and alcohol screen: opiates, methadone, cocaine, amphetamines (including ecstasy), cannabinoids, barbiturates, benzodiazepines, tricyclic antidepressants, and alcohol
Pregnancy test (females only): β-human chorionic gonadotropin in serum

Urine for urinalysis was taken from the PK urine collection container at the end of a collection interval.

In case of unexplained or unexpected clinical laboratory test values, the tests were repeated as soon as possible and followed up until the results had returned to the normal range and/or an adequate explanation for the abnormality was found. The clinical laboratory clearly marked all laboratory test values that were outside the normal range, and the Investigator indicated which of these deviations were clinically significant. Clinically significant laboratory result deviations were recorded as AEs and the relationship to the treatment was indicated.

### Vital Signs

Systolic and diastolic blood pressure and pulse were recorded after the subject had been resting for at least 5 minutes in the supine position. These assessments were made using an automated device. Body temperature and respiratory rate were measured subsequently.

### Electrocardiogram

A standard 12-lead ECG was recorded after the subject had been resting for at least 5 minutes in the supine position. The ECG was recorded using an ECG machine equipped with computer-based interval measurements (with no/minimal disturbance by procedures). The following ECG parameters were recorded: heart rate, PR-interval, QRS-duration, QT-interval, QTcF-interval, and the interpretation of the ECG profile by the Investigator.

### Continuous Cardiac Monitoring (Telemetry)

In the SAD part (not in the second period of the FE group A4, and not in Group A5), a 12-lead ECG was recorded continuously by telemetry from 2 hours before to 24 hours after drug administration on Day 1.

In the MAD part, a 12-lead ECG was recorded continuously by telemetry from 2 hours before to 12 hours after drug administration on Day 1, and from 2 hours before to 24 hours after drug administration on Day 14.

All relevant or significant arrhythmic events were recorded in rhythm strips (10 seconds). The ECG was evaluated by the Investigator for clinically significant events.

During days with telemetry, meals were standardized, and subjects remained quietly supine (with no/minimal disturbance by procedures) for 10 minutes followed by an up to 5-minute period for each ECG assessment that was planned just prior to PK sampling. Start and stop time of the (in total) 15-minute periods were recorded. The ECGs collected by continuous monitoring (telemetry) were stored for potential later use.

These ECGs may or may not be analyzed for the purpose of concentration-effect modeling, based on future development decisions for IMB-1018972. If analyzed, results of the modeling were not to be included in this CSR, but to be included in a separate report.

### Physical Examination

Physical examination was performed according to PRA SOPs. In addition, body weight and height were measured according to PRA SOPs.

### Pharmacokinetic Measurements

### Blood Sampling

At the time points defined in the schedules of assessments, blood samples of 3 mL per time point were taken for the analysis of IMB-1018972, IMB-1028814, and trimetazidine in plasma samples. The blood samples were taken via an indwelling intravenous catheter or by direct venipuncture. The exact times of blood sampling were recorded in the eCRF.

During days with telemetry, subjects remained quietly supine (with no/minimal disturbance by procedures) for 10 minutes followed by an up to 5-minute period for each ECG assessment that was planned just prior to PK sampling. Start and stop time of the (in total) 15-minute periods were recorded.

Details on sample collection, sample aliquoting, sample handling, sample storage, and sample shipping can be found in the laboratory manual prepared by PRA.

Plasma samples may (in the future) also be used for research purposes such as evaluation of the activity of IMB-1018972 and trimetazidine, identification of exploratory biomarkers that are predictive of activity, cytochrome P450 profiling, or other exploratory evaluations that may help characterize the molecular mechanisms of IMB-1018972 and trimetazidine. The samples will be stored for a maximum of 15 years for this purpose.

### Urine Collection

Urine collection for PK was only conducted in the SAD part, but not in the second period of the FE group A4.

During the intervals defined in the schedules of assessments, urine was collected for the analysis of IMB-1018972, IMB-1028814, and trimetazidine. The subjects were instructed to empty their bladders completely before study drug administration and at the end of each collection interval. A blank urine sample was collected within 12 hours prior to study drug administration. The exact times of urine collection and the urine weight of the entire interval (before and after addition of any urine stabilizers, if used) were recorded in the eCRF.

Details on sample collection, sample aliquoting, sample handling, sample storage, and sample shipping can be found in the laboratory manual prepared by PRA.

Urine samples could be kept for a maximum of 1 year for further analysis of metabolites in urine in case unknown metabolites were found in plasma.

### Genotyping

At the time points defined in the schedules of assessments, a blood sample of a maximum of 7 mL was collected for genotyping to better understand the effects of genotype, such as CYP alleles, on PK data. This blood sample was optional for subjects that had already been screened prior to IEC approval of protocol Version 3.0 (25 Mar. 2019), whereas it was mandatory for subjects participating in this study that had been screened after IEC approval of protocol Version 3.0 (25 Mar. 2019).

The blood sample was double coded (1 code at PRA and 1 code at the Sponsor), and the sample was kept until 15 years after completion of the study.

The blood sample was taken via an indwelling intravenous catheter or by direct venipuncture. The exact time of blood sampling was recorded in the eCRF.

Details on sample collection, sample aliquoting, sample handling, sample storage, and sample shipping can be found in the laboratory manual prepared by PRA.

### Safety and Pharmacokinetic Variables

The safety variables to be measured included:
AEs
Clinical laboratory
Vital signs
12-lead ECG
Continuous cardiac monitoring (telemetry)
Physical examination

### Pharmacokinetic Variables

Pharmacokinetic variables were the plasma and urine concentrations of IMB-1018972, IMB-1028814, and trimetazidine, and their PK parameters. The PK parameters that were determined or calculated using noncompartmental analysis are given in Table 3.

TABLE 3
Plasma IMB-1018972, IMB-1028814, and Trimetazidine Parameters

| | | MAD | MAD | |
|---|---|---|---|---|
| Parameter | SAD/FE | Day 1 | Day 14 | Description |
| Cₘₐₓ | X | X | X | Maximum plasma concentration. Observed |
| | | | | peak analyte concentration obtained |
| | | | | directly from the experimental data without |
| | | | | interpolation, expressed in concentration |
| | | | | units. |
| Cₘᵢₙ | | | X | Minimum plasma concentration (predose |
| | | | | concentration excluded). |
| tₘₐₓ | X | X | X | Time to maximum plasma concentration. |
| | | | | First observed time to reach peak analyte |
| | | | | concentration obtained directly from the |
| | | | | experimental data without interpolation, |
| | | | | expressed in time units. |
| AUC₀₋ₜ | X | | | Area under the plasma concentration-time |
| | | | | curve (time 0 to time of last quantifiable |
| | | | | concentration). |
| AUC_{0-inf} | X | | | Area under the plasma concentration-time |
| | | | | curve (time 0 to infinity). Percent |
| | | | | extrapolation less than or equal to 20% is |
| | | | | required to obtain a reliable AUC_{0-inf} |
| % AUCₑₓₜᵣₐ | X | | | Percentage of estimated part of the |
| | | | | calculation of AUC_{0-inf}. Calculated as: |
| | | | | ([AUC_{0-inf} − AUC₀₋ₜ]/AUC_{0-inf})*100%. |
| AUC_{0-τ} | | X | X | Area under the plasma concentration-time |
| | | | | curve over the dosing interval of 0-12 |
| | | | | hours postmorning dose. |
| kₑₗ | X | | X | Terminal elimination rate constant |
| | | | | calculated by linear regression of the |
| | | | | terminal log-linear portion of the |
| | | | | concentration vs time curve. Linear |
| | | | | regression of at least 3 points and an |
| | | | | adjusted r² greater than 0.80 were required |
| | | | | to obtain a reliable kₑₗ. |
| t_{1/2} | X | | X | Terminal elimination half-life |
| | | | | expressed in time units. |
| | | | | Percent extrapolation less than or |
| | | | | equal to 20% and adjusted r² |
| | | | | greater than 0.80 was required to |
| | | | | obtain a reliable t_{1/2}. |
| CL/F | X | | | Apparent oral clearance, |
| | | | | calculated as dose/AUC_{0-inf} IMB-1028814 |
| | | | | only, assuming 100% IMB-1018972 was |
| | | | | converted to IMB-1028814. |
| CLₛₛ/F | | | X | Apparent oral clearance at steady |
| | | | | state, calculated as dose/AUC_{0-τ}. The |
| | | | | AUC_{0-τ} after the morning dose was used in |
| | | | | the calculation. IMB-1028814 only, |
| | | | | assuming 100% IMB-1018972 was |
| | | | | converted to IMB-1028814. |
| V_{z}/F | X | | X | Apparent volume of distribution at terminal |
| | | | | phase, calculated as (CL/F)/kₑₗ |
| | | | | (SAD/FE/MR), or as (CLss/F)/kₑₗ (MAD). |
| | | | | For IMB-1028814 only. |
| R_{ac} | | | X | Accumulation ratio, based on AUC_{0-τ} of |
| | | | | Day 14 vs Day 1 (AUC_{0-τ} after morning |
| | | | | dose). |

FE = food effect;
MAD = multiple ascending dose;
SAD = single ascending dose;
MD = multiple dose;
MR = modified release;
SD = single dose

The sum of IMB-1028814 and trimetazidine concentrations and PK parameters was calculated corrected for molecular weights of 310 kDa for IMB-1028814 and 266 kDa for trimetazidine.

Plasma trough levels of IMB-1018972, IMB-1028814, and trimetazidine were also determined (MAD part only).

The AUCs were calculated using the linear up/log down trapezoidal rule, expressed in units of concentration×time.

TABLE 4
Urine IMB-1018972, IMB-1028814, and Trimetazidine Parameters

| | SAD/FE | |
|---|---|---|
| Parameter | (first period) | Description |
| Aeᵤᵣᵢₙₑ | X | Total amount of drug excreted unchanged into urine to time t |
| | | (time of last measurable concentration), obtained by adding |
| | | the amounts excreted over each collection interval. |
| Feᵤᵣᵢₙₑ | X | Fraction (%) of the administered dose excreted unchanged |
| | | into urine. Calculated as: Feᵤᵣᵢₙₑ = (Aeᵤᵣᵢₙₑ/Dose) * 100. |
| CL_{R} | X | Renal clearance. Calculated as Aeᵤᵣᵢₙₑ/AUC₀₋ₜ. |

### Drug Concentration Measurements

The analysis of IMB1018972, IMB-1028814, and trimetazidine in plasma and urine samples was performed at the Bioanalytical Laboratory of PRA using validated liquid chromatography-mass spectrometry/mass spectrometry methods. The results from calibration samples and quality control samples demonstrated acceptable performance of the methods throughout the experimental period. Data on the performance of the method and stability indicate that the sample results as reported are reliable.

### Statistical and Analytical Plan for Safety and Pharmacokinetic Evolution

### Safety Set

All subjects who had received at least 1 dose of IMB1018972, trimetazidine, or placebo.

### Pharmacokinetic Set

All subjects who had received at least 1 dose of IMB-1018972 or trimetazidine and provided sufficient bioanalytical assessment results to calculate reliable estimates of the PK parameters.

### Statistical and Analytical Plan for Safety and Pharmacokinetic Evaluation

Details on the preparation of the listings and summary tables and figures can be found in the SAP and was generated by the Biostatistics Department of PRA. The SAP was finalized prior to database lock (and unblinding of study treatment codes).

All safety and PK data were listed. In addition, all data were summarized in tabular and/or graphical form and descriptive statistics were given, as appropriate.

### Evolution of Safety and Tolerability

Safety and tolerability were assessed through AEs, clinical laboratory, vital signs, ECGs, continuous cardiac monitoring (telemetry), and physical examination findings, and any other parameter that was relevant for safety assessment.

All individual safety results were listed and descriptive statistics including change from baseline were calculated, where applicable.

### Pharmacokinetic Evaluation

Descriptive statistics (number, arithmetic mean, SD, coefficient of variation, minimum, maximum, median, and geometric mean) were calculated for plasma and urine PK parameters of IMB-1028814, trimetazidine, and IMB-1028814+trimetazidine in the PK population, where applicable.

Dose proportionality of IMB-1018972, IMB-1028814 and trimetazidine was explored for SAD Groups A1 to A4 (fasted) using a regression (power) model relating log-transformed Cmax, AUC₀₋ₜ, and AUC_{0-inf}. Subjects with R2 below 0.80 or % AUCextra>20% were not excluded from the dose-proportionality evaluation based on AUC_{0-inf}. A point estimate and 95% CI were produced for the slope. A slope of 1 (i.e., a 95% CI containing 1) means that no evidence of a deviation from dose proportionality was found. Since there were only 2 dose levels in the MAD part, no dose-proportionality analysis was performed for the MAD part.

The effect of food on the relative oral bioavailability of IMB-1018972 following a single oral administration was explored. This occurred in Group A4 of the SAD part where subjects received the same dose, first under fasted conditions and then under fed conditions. The evaluation was based on 90% CIs for the ratio of the geometric means, based on log-transformed data, for Cₘₐₓ, AUC₀₋ₜ, and AUC_{0-inf}.

### Determination of Sample Size

For this FIH study, no prospective calculations of statistical power were made. The sample size was selected to provide information on safety, tolerability, and PK following single and multiple doses of IMB-1018972, single doses of trimetazidine, and is typical for a FIH study. Any p-values to be calculated according to the SAP were interpreted in the perspective of the explorative character of this study.

### Study Subjects

Of the 220 subjects who were screened, 88 subjects were included in the study and received the study drug. Sixty-six subjects received a dose of IMB-1018972, 8 received trimetazidine, and 14 received placebo.

Eighty-five of 88 subjects completed the study. Subject 129 of the FE arm Group A4 withdrew consent on Day 1 of the second period after receiving the single oral dose of 150 mg IMB-1018972 under fed conditions. Subject 131 of the FE arm Group A4 was withdrawn from the study due to an SAE of influenza like illness (of moderate severity and unlikely related) in the first period and only received the single oral dose of 150 mg IMB-1018972 under fasted conditions and not the fed dose in the second treatment period.

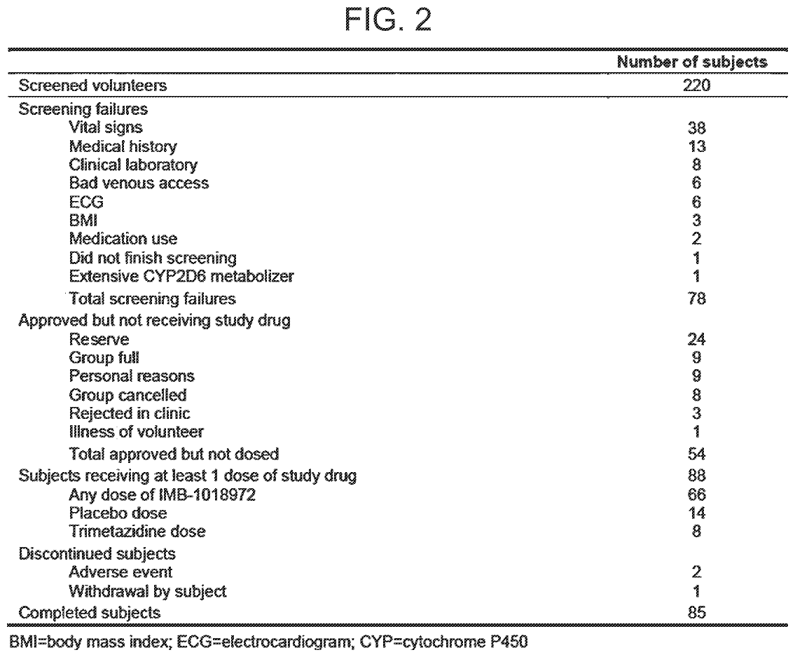

FIG. 2 is a table of the disposition of subjects.

### Protocol Deviations/Violations

Several protocol deviations that were not deemed significant are described in the listings given above; these are not further described in this section.

Two major deviations were recorded in the study:
There was 1 major deviation from the GCP and PRA procedures. Three volunteers were screened 1 day before the study initiation visit was conducted. This deviation was not considered to have had any implications for the safety of the involved volunteers.
The pharmacy at PRA had only received an original set of randomization lists for the SAD part with the treatments as described in Versions 2.0 and 3.0 of the CSP. They did not receive updated randomization lists based on Versions 4.0 and 5.0 of the CSP which changed the designs of Groups A4 and A5. Similarly, the Bioanalytical Laboratory of PRA also did not receive the updated randomization lists from the pharmacy. However, all subjects of Groups A4 and A5 received the correct dose and therefore, this deviation did not have any implications for the safety of the involved volunteers.

In addition, a memo to file, dated 22 Oct. 2019, was issued documenting the following protocol deviations:
For all subjects of Groups B1 and B2 of the MAD part, vital signs on Day 1 at 12 hours after the morning dose were recorded prior to instead of after the ECG and PK blood sampling because of the risk that the evening dose could not be administered in time.
For all subjects of Groups B1 and B2 of the MAD part, vital signs on Day 14 in the morning were scheduled 25 minutes earlier than planned because of the risk that the morning dose could not be administered in time.

### Genotyping

Except for 1 subject in the SAD part, all subjects provided a blood sample for genotyping. The blood sample was used to genotype subjects with a particular interest on CYP2D6 to better understand differences in the PK data. Any results of the analysis of the relationship between genotype and PK data will presented separately from this CSR.

### Measurements of Treatment Compliance

Study drug was administered in the clinical research center. To ensure treatment compliance, administration of the study drug was supervised by the Investigator or authorized designee. There was no indication of noncompliance based on observations during study drug administration. In addition, bioanalytical assessment of IMB-1018972, IMB-1028814, and trimetazidine in plasma and urine samples confirmed treatment compliance.

### Clinical Laboratory Evaluation

### Laboratory Values Over Time

Although several individual changes from baseline were observed in the clinical laboratory values, no clinically important trends were seen.

### Individual Subject Changes

The majority of the subjects had one or more out of range values for clinical laboratory tests at various times during the study. Most of these were minor and considered by the Investigator to have no clinical implication. A number of ALT levels measured for 1 subject were above the normal range and considered to be clinically significant abnormal.

### Vital Signs, ECGs, Physical Findings, and Other Observations Related to Safety

### Vital Signs

Although several individual changes from baseline were observed, blood pressure, pulse, body temperature, and respiratory rate showed no trends or clinically relevant changes during any of the study parts.

### Electrocardiogram

No changes or trends of clinical significance were seen for the heart rate, PR-interval, QRS-duration, QT-interval, or QTcF-interval during any of the study parts. All 12-lead ECG evaluations were recorded as normal or, in case of abnormal recordings, these were not considered to be clinically significant.

### Continuous Cardiac Monitoring (Telemetry)

All telemetric ECG evaluations obtained in the SAD and MAD parts were recorded as normal or, in case of abnormal recordings, these were not considered to be clinically significant.

### Physical Examination

All abnormalities observed at screening and all changes observed after screening for physical examinations were considered to be of no clinical significance.

### Detailed Results for Immediate Release Formulations

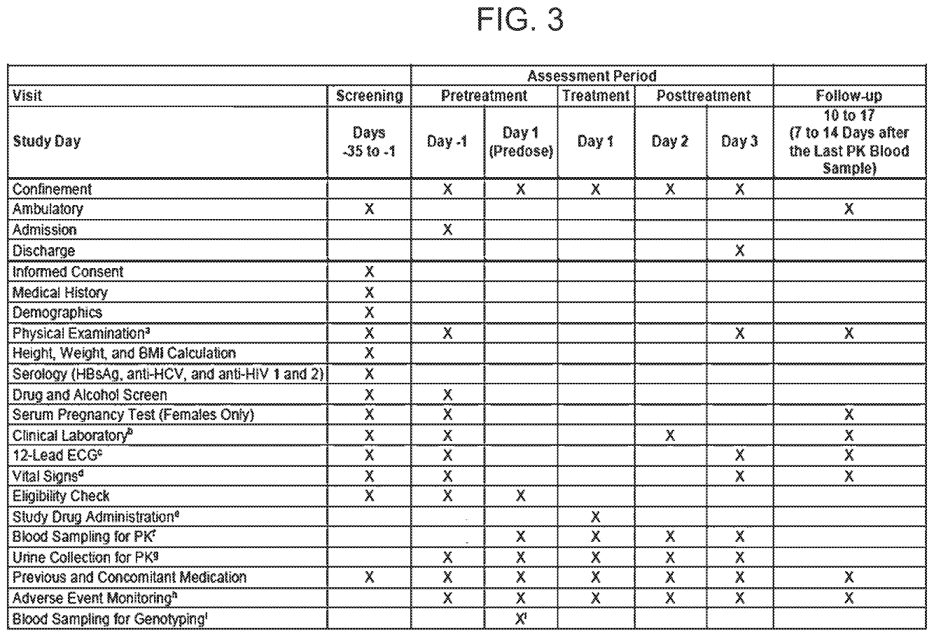

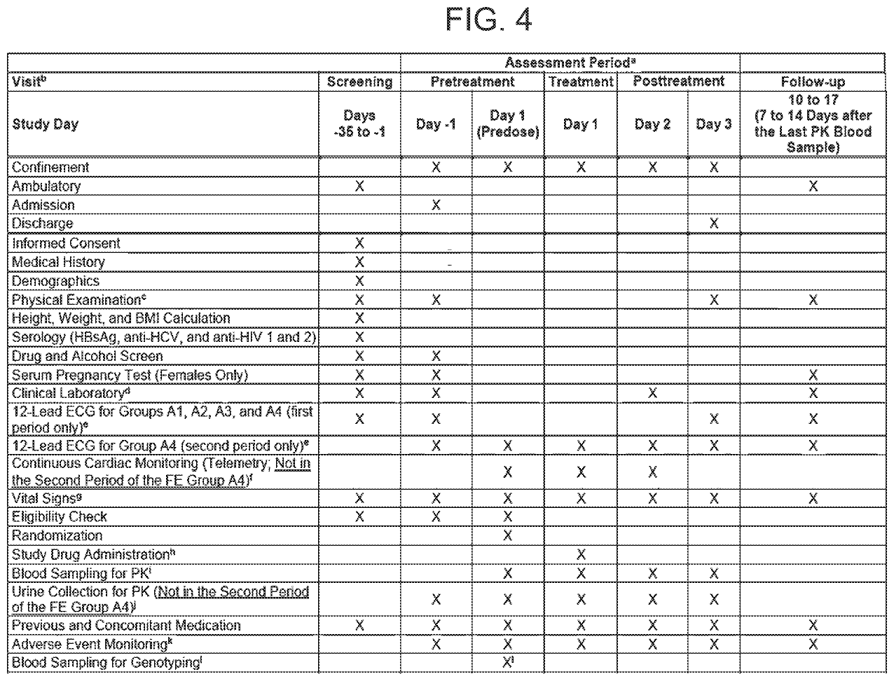

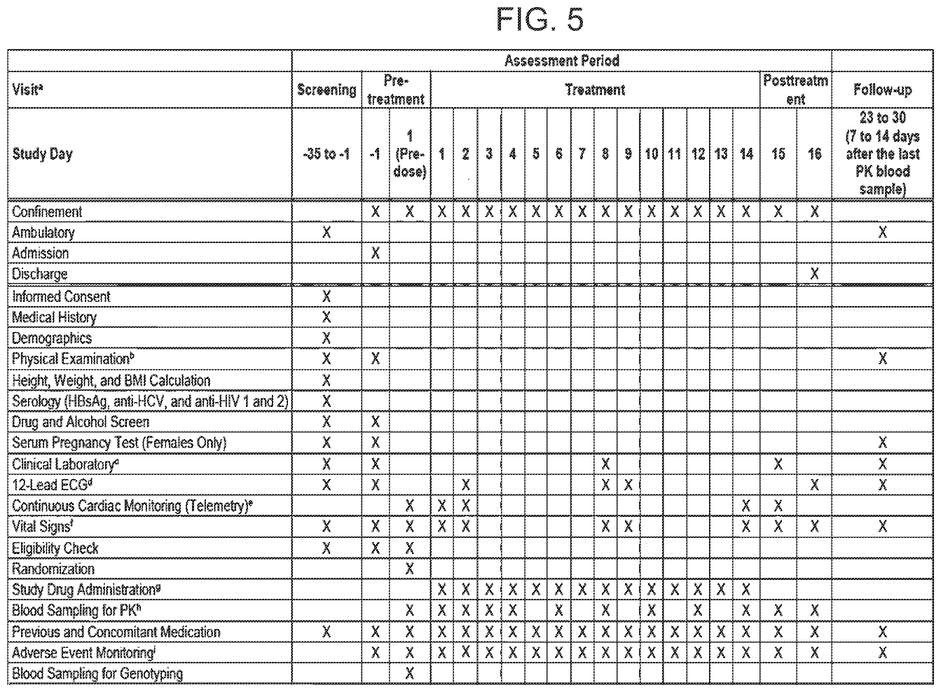

FIG. 3 is a Schedule of Assessments for SAD part Group A5, with the following notations:
BMI-body mass index;
ECG-electrocardiogram;
HBsAg-hepatitis B surface antigen;
HCV=hepatitis C virus;
PK-pharmacokinetic(s)
a. Physical examination: at screening, on Day −1 (admission; this was a directed examination only done at the discretion of the Investigator), at discharge on Day 3 (this was a directed examination only done at the discretion of the Investigator), and at follow-up.
b. Clinical laboratory tests (including clinical chemistry, hematology, coagulation, and urinalysis): at screening, on Day −1 (admission) and at 24 hours postdose, and at follow-up.
c. 12-lead ECG at screening, on Day −1 (admission), at 48 hours postdose, and at follow-up.
d. Vital signs (supine systolic and diastolic blood pressure, pulse, body temperature, and respiratory rate): at screening, on Day −1 (admission), at 48 hours postdose, and at follow-up.
e Study drug administration was conducted under fasted conditions
   FIG. 4 is a table of assessments given for the SAD part (and integrated FE arm) Groups A1 to A4, with the following notations:
BMI-body mass index
ECG=electrocardiogram;
FDA=Food and Drug Administration;
FE-food effect;
HBsAg-hepatitis B surface antigen;
HCV=hepatitis C virus;
PK=pharmacokinetic(s); SAD=single ascending dose
a. Subjects were in the clinic for 1 period, except for subjects of Group A4 also participating in the FE arm who were in the clinic for 2 periods; a period was from Day-1 until 48 hours (Day 3) postdose.
b. The planned confinement period, day of discharge, and follow-up period could be adapted depending on emerging study results. Also, the timing, type, and number of safety and PK assessments could be changed during the study.
c. Physical examination: at screening, each period on Day −1 (admission; this was a directed examination only done at the discretion of the Investigator), at discharge on Day 3 (this was a directed examination only done at the discretion of the Investigator), and at follow-up.
d. Clinical laboratory tests (including clinical chemistry, hematology, coagulation, and urinalysis): at screening, each period on Day −1 (admission) and at 24 hours postdose, and at follow-up.
e. 12-lead ECG for Groups A1, A2, A3, and A4 (first period only): at screening, on Day −1 (admission), at 48 hours postdose, and at follow-up. Data for 12-lead ECGs at predose and 1, 2, 4, 6, 12, and 24 hours postdose were taken from the 12-lead ECG prints from telemetry. The predose baseline value was the average values of the 3 predose telemetry 12-lead ECGs at −1.25, −1.0, and −0.75 hours predose. 12-lead ECG for Group A4 (second period only): on Day −1 (admission), at predose and 1, 2, 4, 6, 12, 24, and 48 hours postdose, and at follow-up.
f. Only in the SAD part; not in the second period of Group A4 also participating in the FE arm: Continuous cardiac monitoring (12-lead telemetry): from at least 2 hours predose until at least 24 hours postdose. 12-lead ECG reads were printed at −1.25, −1.0, and −0.75 hours predose and just prior to the PK sampling time points of 0.25, 0.5, 1, 2, 4, 6, 12, and 24 (Day 2) hours postdose.
g. Vital signs (supine systolic and diastolic blood pressure, pulse, body temperature, and respiratory rate): at screening, each period on Day −1 (admission), each period at predose and 1, 2, 4, 6, 12, 24, and 48 hours postdose, and at follow-up.
h In Groups A1, A2, A3, and, A4, and in the first period of Group A4 also participating in the FE arm, study drug administration was conducted under fasted conditions. In the second period of Group A4, drug administration was conducted under fed conditions (FDA-defined high-fat breakfast).
i. Blood sampling for PK of IMB-1018972, IMB-1028814, and trimetazidine in plasma: each period at predose and 0.25, 0.5, 1, 2, 3, 4, 5, 6, 8, 10, 12, 16, 24, 36, and 48 hours postdose.
j. Only in the SAD part; not in the second period of Group A4 also participating in the FE arm: Urine collection for PK of IMB-1018972, IMB-1028814, and trimetazidine in urine: each period at predose (within 12 hours prior to dosing) and over 0-6, 6-12, 12-24, 24-36, and 36-48 hours postdose collection intervals.
k. AEs were recorded from (first) admission until completion of the follow-up visit. 1 Blood sampling for genotyping was optional for subjects that had already been screened prior to IEC approval of protocol Version 3.0 (25 Mar. 2019), whereas it was mandatory for subjects participating in this study that had been screened after IEC approval of protocol Version 3.0 (25 Mar. 2019). For the subjects for which this sample was mandatory, the sample was taken on Day 1 of the first period only. For the subjects for which this sample was optional and who consented to provide this sample, the sample could be taken on any day during the study; a separate visit could be planned after follow-up to take this sample, if needed.
   FIG. 5 is a table of assessments given for the MAD part, with the following notations:
BMI-body mass index;
ECG=electrocardiogram;
FE-food effect;
HBsAg-hepatitis B surface antigen;
HCV=hepatitis C virus;
MAD=multiple ascending dose;
PK=pharmacokinetic(s);
q8 h=every 8 hours; q12 h=every 12 hours; qd=once daily;
SAD=single ascending dose; tid-three times a day
a. The planned confinement period, day of discharge, and follow-up period could be adapted depending on emerging study results. Also, the timing, type, and number of safety and PK assessments could be changed during the study.
b. Physical examination: at screening, on Day −1 (admission; this was a directed examination only done at the discretion of the Investigator), and at follow-up. On other days, a physical examination could be done on indication only at the discretion of the Investigator.
c. Clinical laboratory tests (including clinical chemistry, hematology, coagulation, and urinalysis): at screening, on Day −1 (admission), before the morning dose on Day 8 and at the same time on Day 15, and at follow-up.
d. 12-lead ECG: at screening, on Day −1 (admission), on Day 1 at 24 hours after the morning dose, on Day 8 at predose and 1, 2, 4, 6, 12 (prior to the evening dose) and 24 hours after the morning dose, on Day 16 (day of discharge) at the same time as before the morning dose on dosing days, and at follow-up. Data for 12-lead ECGs on Day 1 at predose and 1, 2, 4, 6, and 12 hours postdose, and on Day 14 at predose and 1, 2, 4, 6, 12, and 24 hours postdose were taken from the 12-lead ECG prints from telemetry. The predose baseline value on Day 1 and Day 14 was the respective average values of the 3 predose telemetry 12-lead ECGs at −1.25, −1.0, and −0.75 hours predose.
e. Continuous cardiac monitoring (12-lead telemetry): from at least 2 hours before the morning dose until at least 12 hours after the morning dose on Day 1 and until at least 24 hours after the morning dose on Day 14. 12-lead ECG reads were printed at −1.25, −1.0, and −0.75 hours before the morning dose and just prior to the PK sampling time points of 0.25, 0.5, 1, 2, 4, 6, 12, and 24 (Day 14 only) hours after the morning dose.
f. Vital signs (supine systolic and diastolic blood pressure, pulse, body temperature, and respiratory rate): at screening, on Day −1 (admission), on Days 1, 8, and 14 at predose and 1, 2, 4, 6, 12 (prior to the evening dose on Days 1 and 8) and 24 hours after the morning dose, on Day 16 (day of discharge) at the same time as before the morning dose on dosing days, and at follow-up.
g. The study drug was administered twice daily for 14 days; on Day 14 only a single morning dose was administered. Study drug administration was conducted under fed conditions as determined based on the results of Group A4 in the FE arm. Note: The study drug was given for 14 consecutive days, but this could be revised based on the safety and tolerability results (and plasma PK results, if available) of the SAD part and of previous group(s) in the MAD part. Similarly, it could be decided to change q12 h dosing to qd or tid (q8 h) dosing.
h. Blood sampling for PK of IMB-1018972, IMB-1028814, and trimetazidine in plasma: on Days 1 and 14 before the morning dose and at 0.25, 0.5, 1, 2, 3, 4, 5, 6, 8, 10, 12 (prior to the evening dose on Day 1), 16, 24, 36, and 48 hours after the morning dose, and on Days 4, 6, 8, 10, and 12 before the morning dose.
i. AEs were recorded from admission until completion of the follow-up visit.
j. Blood sampling for genotyping was mandatory.

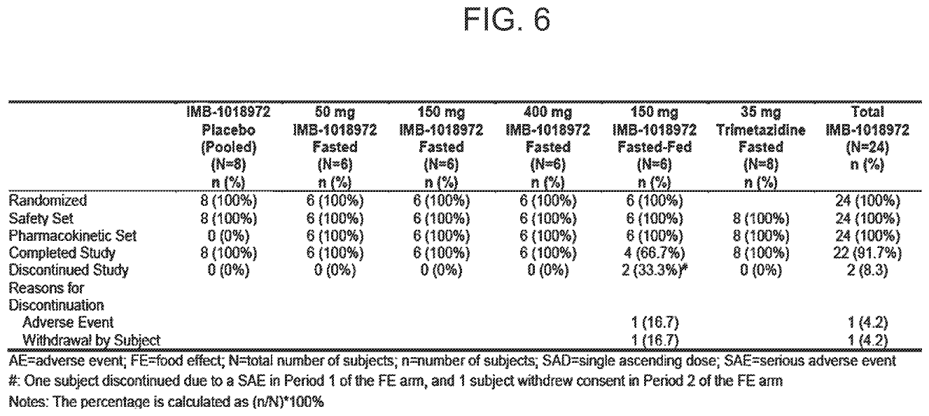

FIG. 6 is a table of analysis data sets for the SAD Part (and integrated FE Arm) per dose level and total for IMB-1018972.

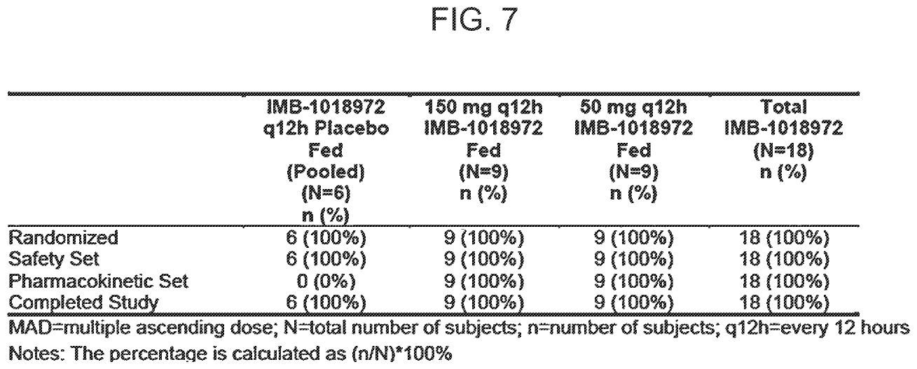

FIG. 7 is a table of analysis data sets for the MAD Part per dose level and total for IMB-1018972.

### Demographic and Other Baseline Characteristics

### SAD Part (and Integrated FE Arm)

In the SAD part with integrated FE arm, a total of 40 subjects were included.

### IMB-1018972 and Placebo

Thirty-two subjects were included of whom 23 were female and 9 were male Mean age ranged between 29 and 46 years and mean BMI ranged between 23.0 and 26.6 kg/m2 over all treatments, including placebo. Individual age ranged between 18 and 65 years and individual BMI ranged between 19.5 and 30.3 kg/m2. Twenty-nine subjects were of white race, 1 subject was Asian, 1 subject was Black or African American, and 1 subject was Native Hawaiian or Other Pacific Islander. Thirty-one subjects were not of Hispanic or Latino ethnicity whereas 1 subject was of Hispanic or Latino ethnicity. The summary of the PK set was identical to that of the safety set minus the pooled placebo group.

### Trimetazidine Group

Eight subjects were included of whom 5 were female and 3 were male. Mean age was 32 years and mean BMI was 23.7 kg/m2. Individual age ranged between 20 and 65 years and individual BMI ranged between 19.4 and 26.7 kg/m2. Seven subjects were of white race and 1 subject was of multiple race. Seven subjects were not of Hispanic or Latino ethnicity whereas 1 subject was of Hispanic or Latino ethnicity. The summary of the PK set was identical to that of the safety set.

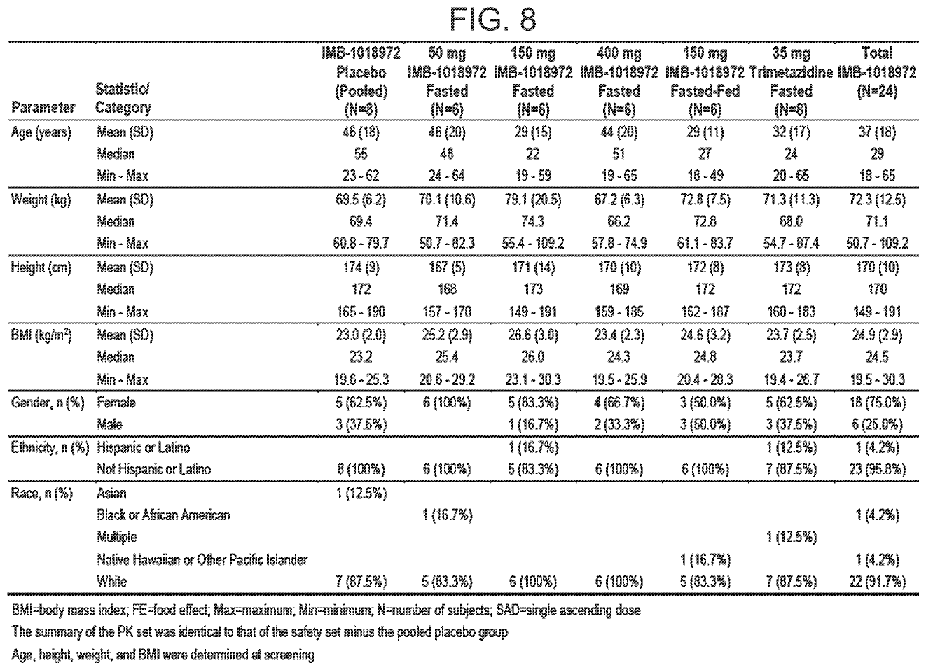

FIG. 8 is a table of a summary of demographic characteristics—SAD Part (and Integrated FE Arm) (Safety Set).

### MAD Part

Twenty-four subjects were included of whom 12 were female and 12 were male. Mean age ranged between 38 and 44 years and mean BMI ranged between 25.2 and 26.7 kg/m2 over all treatments, including placebo. Individual age ranged between 18 and 64 years and individual BMI ranged between 19.1 and 30.9 kg/m2. Eighteen subjects were of white race, 2 subjects were of multiple race, 2 subjects were American Indian or Alaska Native, 1 subject was Asian, and 1 subject was Black or African American. Twenty-one subjects were not of Hispanic or Latino ethnicity whereas 3 subjects were of Hispanic or Latino ethnicity. The summary of the PK set was identical to that of the safety set minus the pooled placebo group.

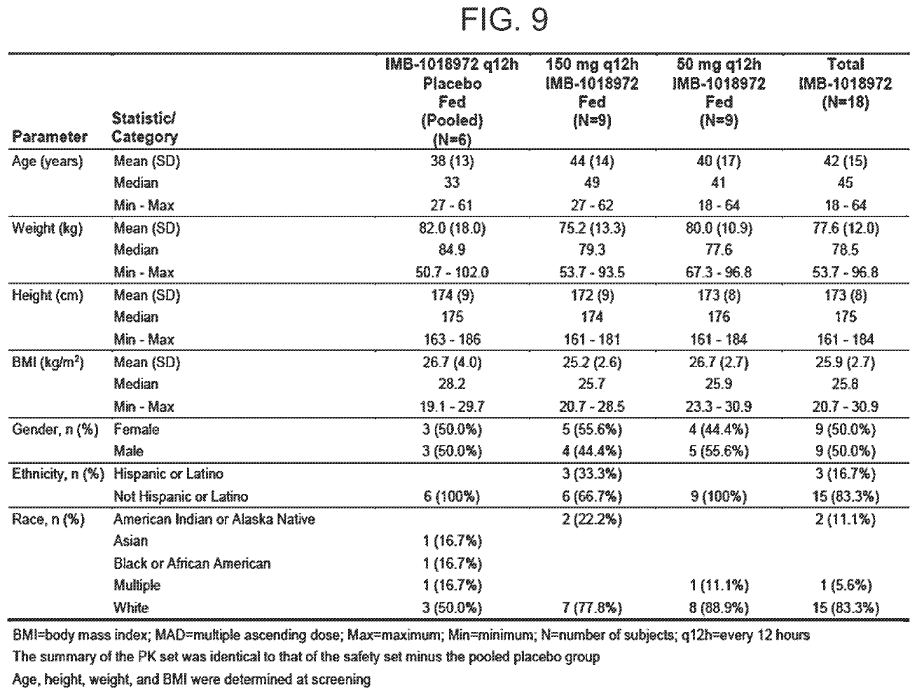

FIG. 9 is a table of a summary of demographic characteristics—MAD Part (Safety Set).

### Other Baseline Characteristics

All subjects complied with the inclusion and exclusion criteria. There were no clinically significant findings with regard to medical history or previous medication. Drug and alcohol screen results were negative for all subjects at screening and (each) admission. The results for the serology parameters were negative at screening for all subjects. The pregnancy test results were negative at screening, (each) admission, and follow-up for all females participating in this study.

### Extent of Exposure

A total of 88 subjects were dosed in this study: 40 subjects in the SAD part with integrated FE arm, 24 subjects in the MAD part.

In each of Groups A1, A2, A3 and A4 of the SAD part, 6 subjects received a single dose of IMB-1018972 and 2 subjects received a single dose of matching placebo under fasted conditions. IMB-1018972 doses ranged from 50 mg to 400 mg over these 4 groups. Subjects of SAD Groups A1, A2, and A3 participated in 1 single-dose treatment period, and subjects of SAD Group A4 (the FE group) participated in 2 single dose treatment periods with fasted dosing in the first period and fed dosing in the second period. Subject 131 of FE Group A4 only received the fasted IMB-1018972 dose in the first treatment period and not the fed dose in the second treatment period since the subject was withdrawn from the study in the first period due to a moderate SAE of influenza like illness (unlikely related).

In Group A5 of the SAD part, 8 subjects received a single oral dose of 35 mg trimetazidine under fasted conditions.

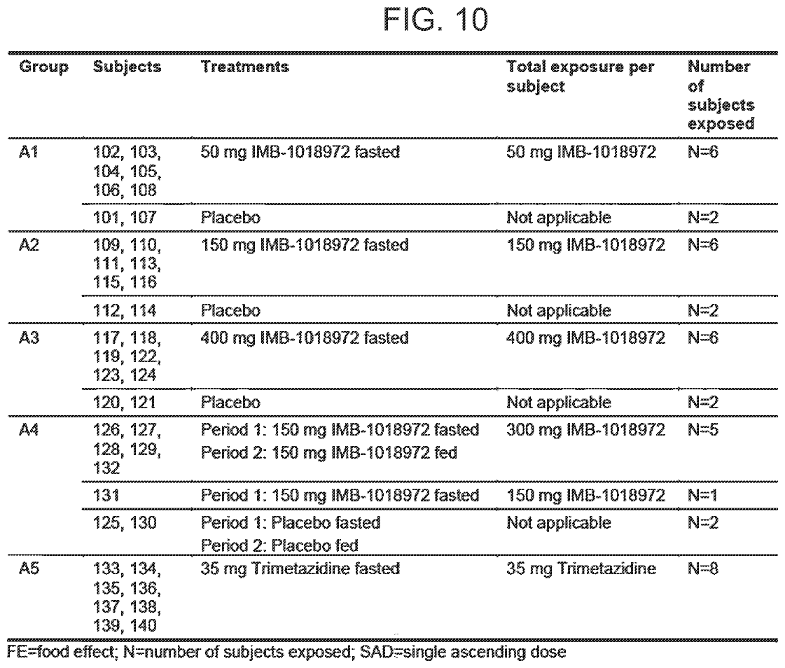

FIG. 10 is a table of the Extent of Exposure—SAD Part (and Integrated FE Arm) (Safety Set)

In both groups of the MAD part, 9 subjects received IMB-1018972 (150 mg for Group B1 and 50 mg for Group B2) and 3 subjects received matching placebo under fed conditions. In both groups, multiple oral doses of IMB-1018972 or matching placebo were administered q12 h on Days 1 to 13 followed by a single morning dose on Day 14.

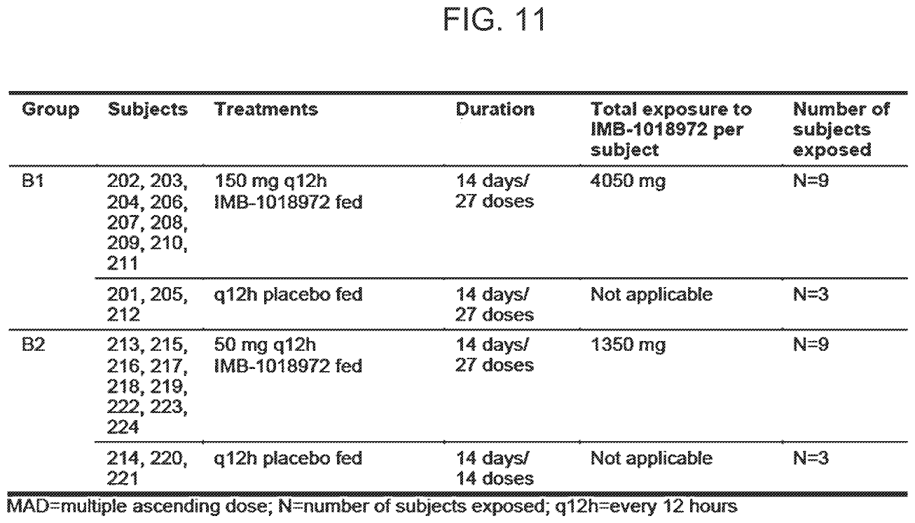

FIG. 11 is a table of the Extent of Exposure—MAD Part

### Pharmacokinetic Evaluation

The lower limit of quantification (LLOQ) was 0.5 ng/ml for IMB-1018972, IMB-1028814 and trimetazidine plasma concentrations, 10 ng/mL for IMB-1028814 urine concentrations, and 50 ng/ml for trimetazidine urine concentrations.

When more than 50% of the plasma values at a particular time point were below LLOQ, geometric means were not determined.

All blood samples of subjects that received IMB-1018972 in this study were analyzed for IMB-1018972 in plasma, but IMB-1018972 could be measured in only few plasma samples. Therefore, the IMB-1018972 concentrations have only been listed and no descriptive statistics or concentration-time profiles have been presented in this CSR. In addition, no PK parameters have been calculated for plasma IMB-1018972. As a result, urine samples were not analyzed for IMB-1018972 concentrations. Since the pharmacodynamic effect of IMB-1028814 and trimetazidine is the same, data are presented for IMB-1028814 and trimetazidine individually, as well as for the sum of IMB-1028814 and trimetazidine concentrations. The sum of IMB-1028814 and trimetazidine concentrations and PK parameters was calculated corrected for molecular weights of 310 kDa for IMB-1028814 and 266 kDa for trimetazidine.

### SAD Part (and Integrated FE Arm)

### PK in Plasma Following Administration of IMB-1018972 Under Fasted Conditions

All predose samples were below the LLOQ for IMB-1028814 and trimetazidine plasma concentrations.

The geometric mean concentration-time profiles for IMB-1028814, metabolite trimetazidine, and IMB-1028814+trimetazidine showed a clear dose-dependent increase in plasma concentrations following administration of single doses of IMB-1018972 under fasted conditions in the dose range of 50 mg to 400 mg IMB-1018972.

The initial hydrolysis of IMB-1018972 to IMB-1028814 and subsequent systemic bioavailability of IMB-1028814 was relatively rapid with detectable concentrations generally seen between 15 and 30 minutes postdose. Detectable concentrations for trimetazidine also generally appeared between 15 and 30 minutes postdose. Median tₘₐₓ was around 1 hour postdose for IMB-1028814, and between 1.5 hours and 2 hours postdose for trimetazidine over the studied single-dose range of 50 mg to 400 mg IMB-1018972 under fasted conditions. Median tₘₐₓ did not increase with increasing IMB-1018972 dose.

The geometric mean Cₘₐₓ increased with dose and ranged between 104 ng/ml and 870 ng/mL for IMB-1028814, between 36.9 ng/ml and 274 ng/mL for trimetazidine, and between 516 nmol/L and 3,839 nmol/L (molar units to account for differences in molecular weight) for IMB-1028814+trimetazidine over the studied single-dose range of 50 mg to 400 mg IMB-1018972 under fasted conditions. Similarly, the geometric mean AUC0-t increased with dose and ranged between 290 ng·h/mL and 2,795 ng·h/mL for IMB-1028814, between 424 ng·h/mL and 3,305 ng·h/mL for trimetazidine, and between 2,970 nmol·h/L and 22,365 nmol·h/L for IMB-1028814+trimetazidine over the studied single-dose range of 50 mg to 400 mg IMB-1018972 under fasted conditions. The predefined stopping criterion for IMB-1028814 plasma exposure of 417,733 and 652,849 ng·h/mL for males and females, respectively, was not reached by any of the subjects during the SAD part.

Elimination of IMB-1028814 took place in a biphasic fashion, whereas elimination of trimetazidine occurred in a monophasic fashion. The geometric mean t_{1/2} of IMB-1028814 was relatively short, ranging between 2.6 hours and 3 hours over the IMB-1018972 single-dose range under fasted conditions. For metabolite trimetazidine, geometric mean t1/2 was longer, ranging between 6.76 hours and 8 hours over the IMB-1018972 single-dose range under fasted conditions. Geometric mean t_{1/2} of IMB-1028814 and trimetazidine did not increase with increasing IMB-1018972 dose indicating that the PK of the 2 moieties was linear.

Detectable individual IMB-1028814 concentrations were observed until 10, 12, 16, or 24 hours postdose after 50 mg, and until 16 or 24 hours postdose after 150 mg and 400 mg IMB-101897. Detectable individual trimetazidine concentrations were observed until 24, 36, or 48 hours postdose after 50 mg, until 36 or 48 hours postdose after 150 mg, and until 48 hours postdose after 400 mg IMB-1018972.

An aberrant IMB-1028814 and trimetazidine concentration-time profile was observed for Subject 108 who had received a single oral dose of 50 mg IMB-1018972 under fasted conditions. IMB-1028814 and trimetazidine tₘₐₓ was much later for this subject (5.00 hours for IMB-1028814 and 8.00 hours for trimetazidine) than for the other subjects who received the same dose (between 0.50 and 1.02 hours for IMB-1028814 and between 1.00 and 2.00 hours for trimetazidine). Therefore, absorption of IMB-1018972 by this subject is much slower than for the other subjects who received the same dose.

Dose proportionality for IMB-1028814 and trimetazidine was explored by plotting the dose-normalized exposure parameters Cₘₐₓ, AUC₀₋ₜ, and AUC_{0-inf} on a linear scale. The 95% CIs of the slopes of all 3 exposure parameters included 1 for both IMB-1028814 and trimetazidine. This indicates that no evidence of a deviation from dose proportionality of IMB-1028814 and trimetazidine was found over the IMB-1018972 single-dose range of 50 to 400 mg.

### PK in Plasma Following Administration of Trimetazidine

All predose samples were below the LLOQ for trimetazidine plasma concentrations. Following administration of a single oral dose of 35 mg trimetazidine, detectable trimetazidine concentrations were generally seen between 15 and 30 minutes postdose. Median trimetazidine tₘₐₓ was 5 hours, and geometric mean values were 68.6 ng/ml for Cₘₐₓ, 912 ng·h/mL for AUC0-t, and 929 ng·h/mL for AUC_{0-inf}.

Elimination of trimetazidine occurred in a monophasic fashion up to the last time point above LLOQ with a geometric mean t_{1/2} of 7.49 hours. Detectable individual trimetazidine concentrations were observed until the last sampling time point of 48 hours postdose.

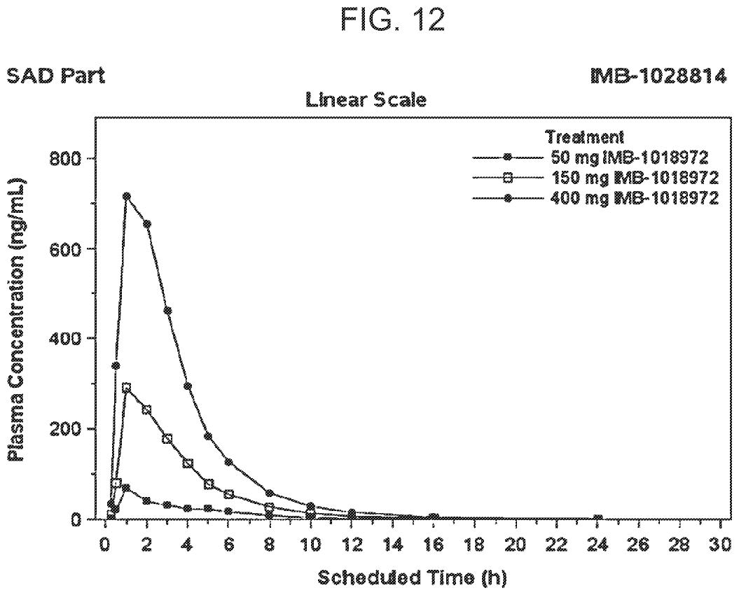

FIG. 12 is a graph of Geometric Mean IMB-1028814 Plasma Concentration-Time Profiles (Linear)—SAD Part (PK Set)

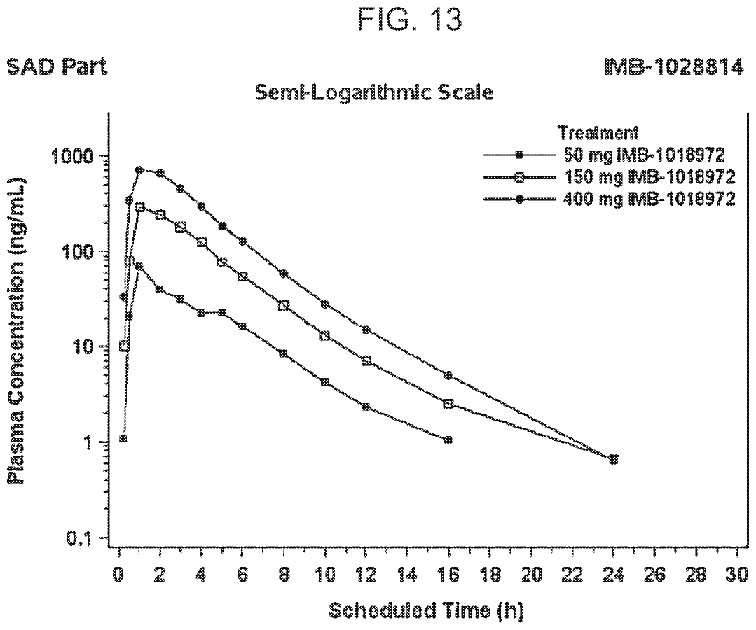

FIG. 13 is a graph of Geometric Mean IMB-1028814 Plasma Concentration-Time Profiles (Semi-Logarithmic)—SAD Part (PK Set)

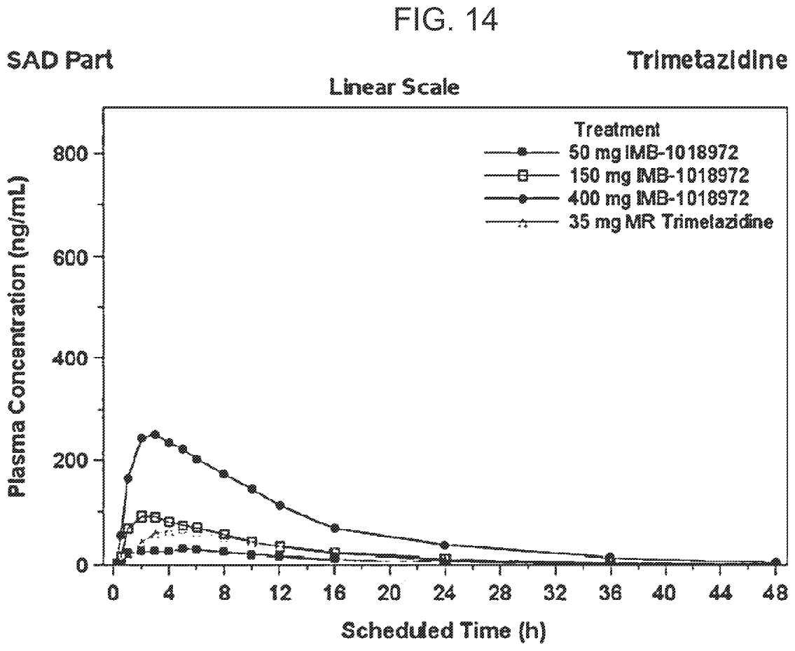

FIG. 14 is a graph of Geometric Mean Trimetazidine Plasma Concentration-Time Profiles (Linear)—SAD Part (PK Set)

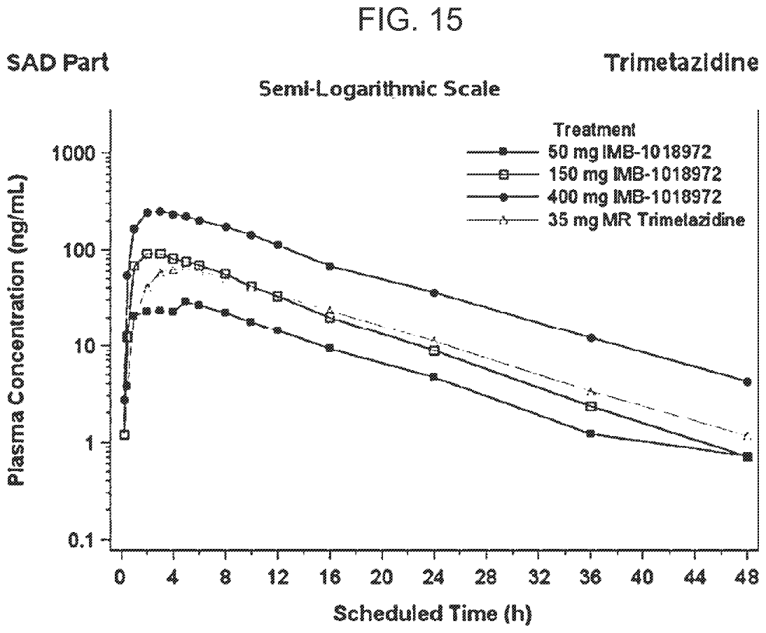

FIG. 15 is a graph of Geometric Mean Trimetazidine Plasma Concentration-Time Profiles (Semi-Logarithmic)—SAD Part (PK Set)

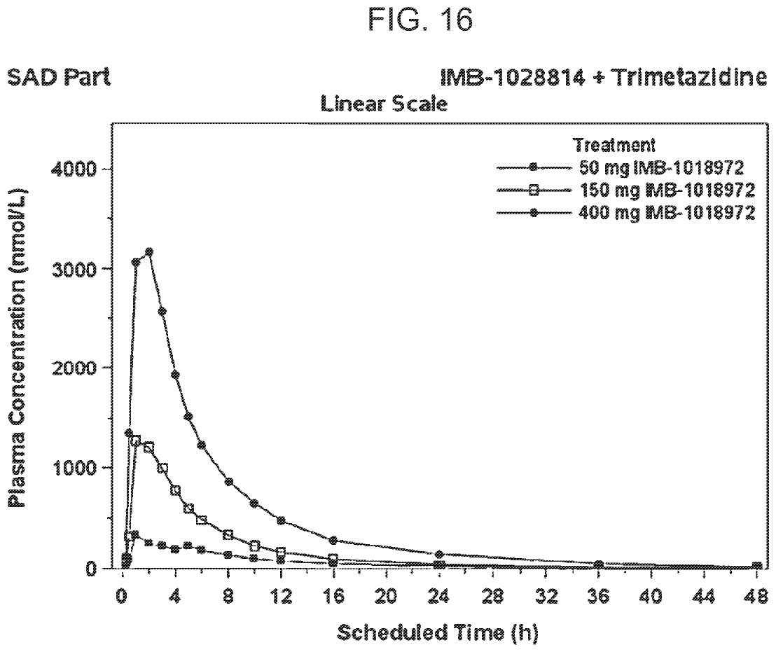

FIG. 16 is a graph of Geometric Mean IMB-1028814+Trimetazidine Plasma Concentration-Time Profiles (Semi-Logarithmic)—SAD Part (PK Set)

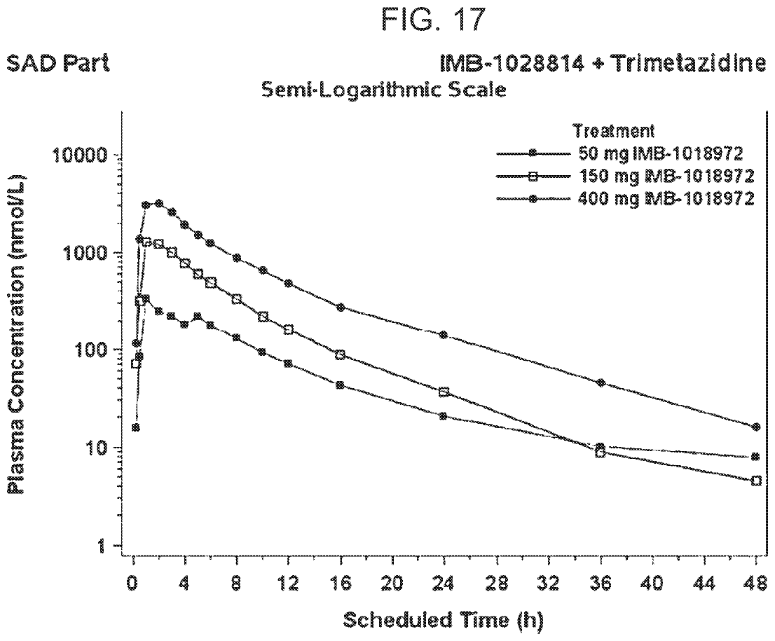

FIG. 17 is a graph of Geometric Mean IMB-1028814+Trimetazidine Plasma Concentration-Time Profiles (Semi-Logarithmic)—SAD Part (PK Set)

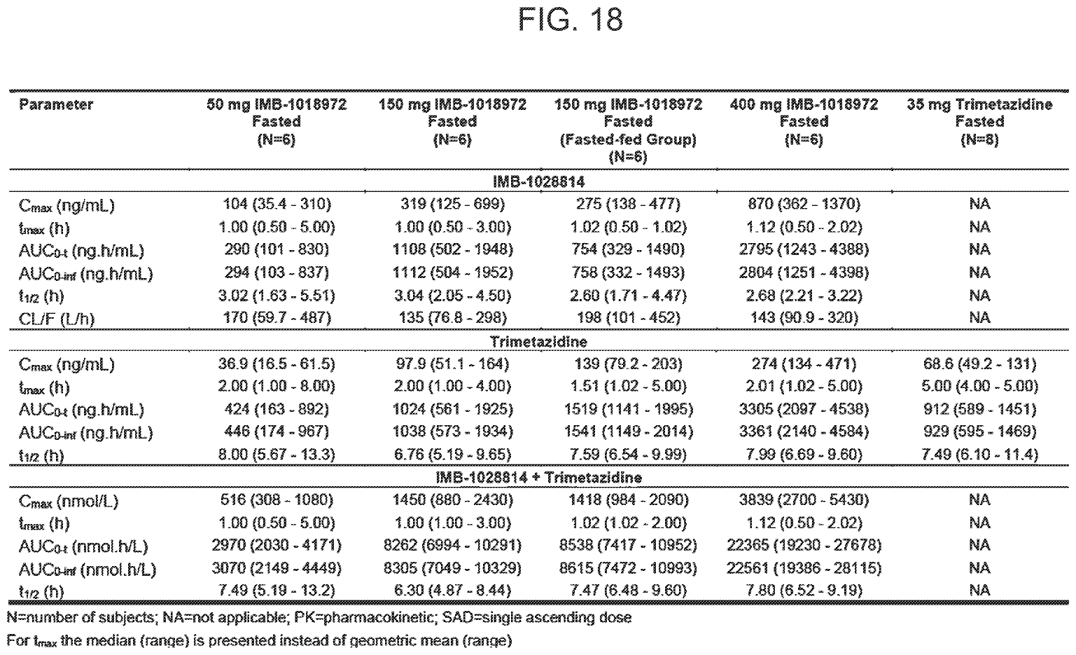

FIG. 18 is a table of Summary Statistics (Geometric Mean [Range]) of IMB-1028814, Trimetazidine, and IMB-1028814+Trimetazidine Plasma Pharmacokinetic Parameters—SAD Part (PK Set)

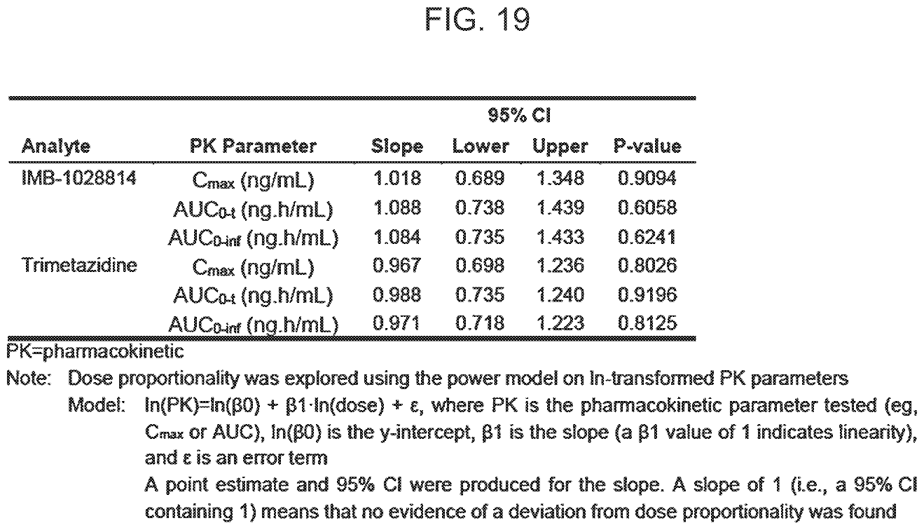

FIG. 19 is a table of Exploratory Analysis of Dose Proportionality for IMB-1028814 and Trimetazidine over the Dose Range of 50 mg to 400 mg IMB-1018972 under Faster Conditions—SAD Part (PK Set)

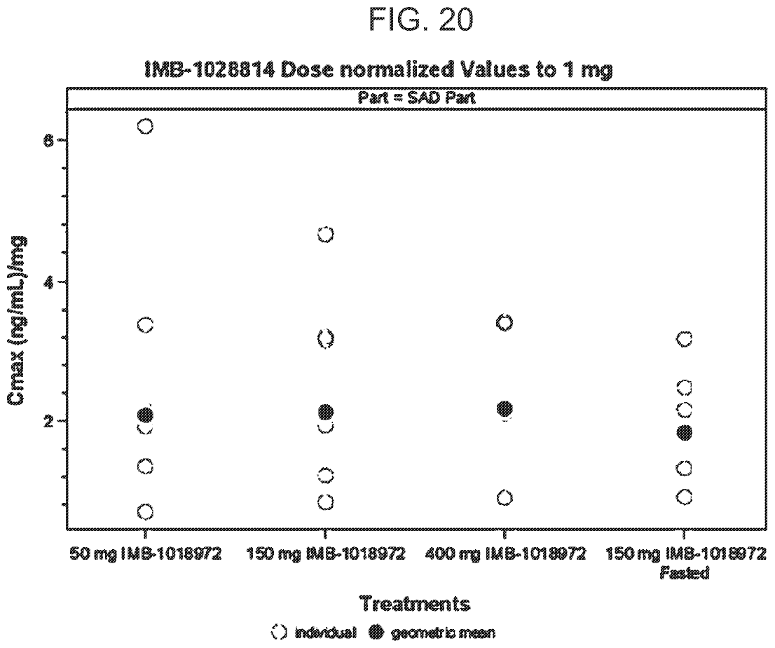

FIG. 20 is a graph of Plot of Combined Individual and Geometric Mean Dose-Normalized IMB-1028814 Cₘₐₓ over the Dose Range of 50 mg to 400 mg IMB-1018972 under Fasted Conditions—SAD Part (PK Set)

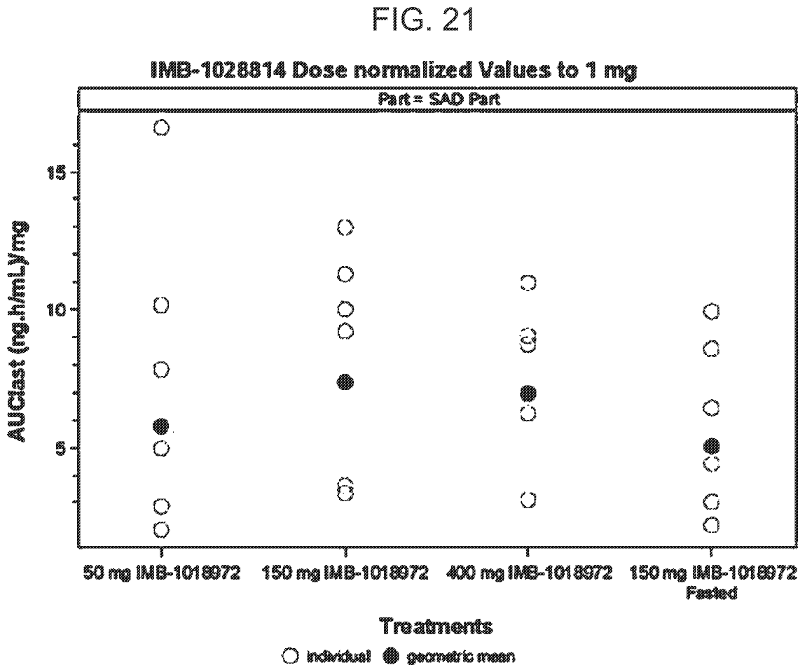

FIG. 21 is a graph of Plot of Combined Individual and Geometric Mean Dose-Normalized IMB-1028814 AUC₀₋ₜ over the Dose Range of 50 mg to 400 mg IMB-1018972 under Fasted Conditions—SAD Part (PK Set)

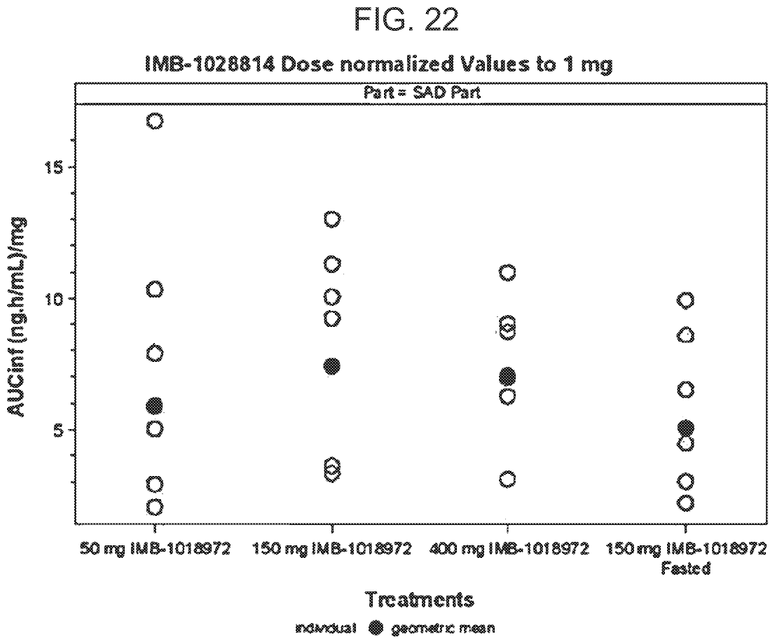

FIG. 22 is a graph of Plot of Combined Individual and Geometric Mean Dose-Normalized IMB-1028814 AUC_{0-inf} over the Dose Range of 50 mg to 400 mg IMB-1018972 under Fasted Conditions—SAD Part (PK Set)

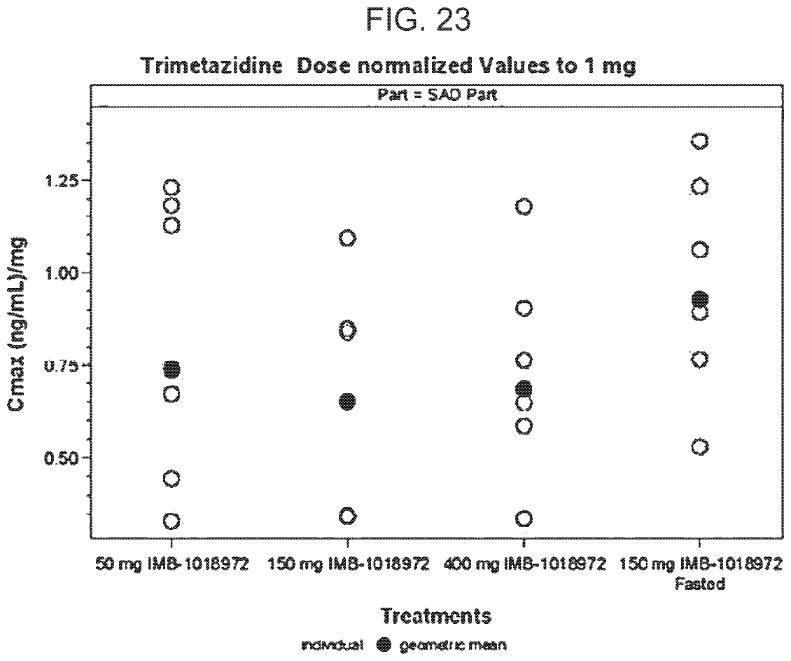

FIG. 23 is a graph of Plot of Combined Individual and Geometric Mean Dose-Normalized Trimetazidine Cₘₐₓ over the Dose Range of 50 mg to 400 mg IMB-1018972 under Fasted Conditions—SAD Part (PK Set)

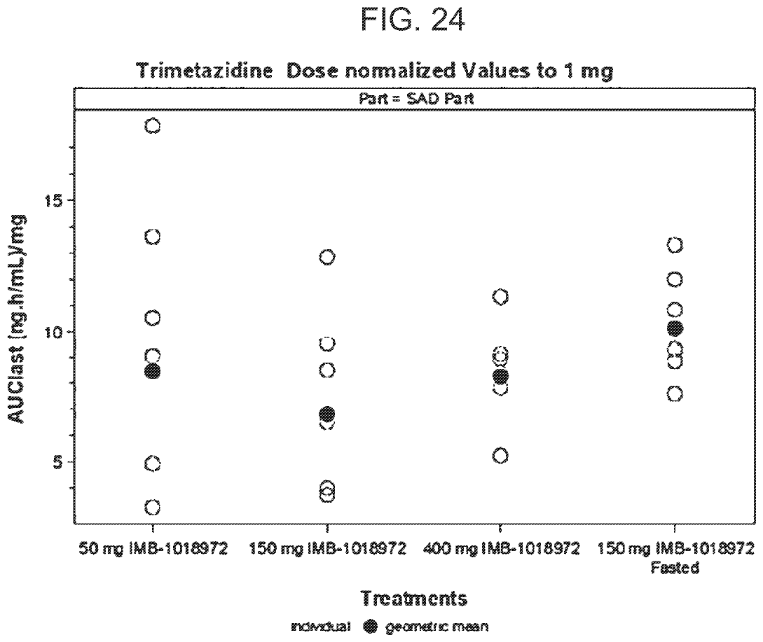

FIG. 24 is a graph of Plot of Combined Individual and Geometric Mean Dose-Normalized Trimetazidine AUC₀₋ₜ over the Dose Range of 50 mg to 400 mg IMB-1018972 under Fasted Conditions—SAD Part (PK Set)

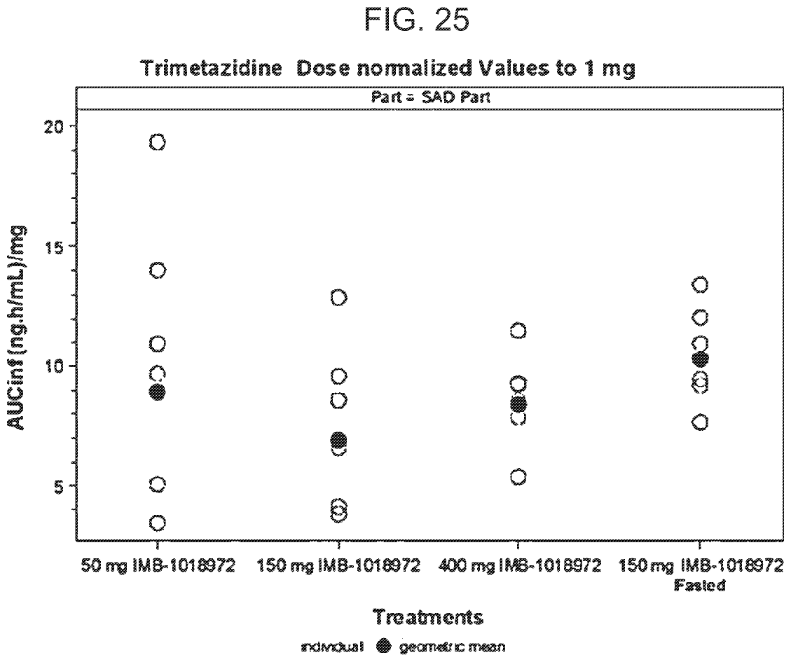

FIG. 25 is a graph of Plot of Combined Individual and Geometric Mean Dose-Normalized Trimetazidine AUC_{0-inf} over the Dose Range of 50 mg to 400 mg IMB-1018972 under Fasted Conditions—SAD Part (PK Set)

### Effect of Food

The possible effect of food on the PK of IMB-1028814 and trimetazidine was explored by comparing administration of single oral doses of 150 mg IMB-1018972 after an FDA-defined high-fat breakfast and under fasted conditions.

All predose samples were below the LLOQ for IMB-1028814 and trimetazidine plasma concentrations.

After study drug administration under fed conditions, the geometric mean IMB-1028814 plasma concentrations initially increased at the same speed as under fasted conditions but then a plateau was reached. When looking at individual profiles, no plateau was observed, but individual IMB-1028814 tmax values ranged between 0.42 and 5 hours. Median tmax was reached at 2 hours postdose under fed conditions relative to 1 hour postdose under fasted conditions.

The trimetazidine plasma concentrations under fed conditions increased less rapidly than after study drug administration under fasted conditions and median tₘₐₓ was reached at 4 hours postdose under fed conditions relative to 1.5 hours postdose under fasted conditions

No evidence for an effect of food was observed on the IMB-1028814 exposure parameters AUC₀₋ₜ and AUC_{0-inf} (both with an estimate of 1.12 and 90% CI ranging from 1.02 to 1.23). However, Cₘₐₓ was approximately 36% lower following administration of a single dose of 150 mg IMB-1018972 after an FDA-defined high-fat breakfast relative to administration under fasted conditions (estimate of 0.64; 90% CI ranging from 0.39 to 1.04).

No evidence for an effect of food was observed on the trimetazidine exposure parameters Cmax (estimate of 0.91; 90% CI ranging from 0.85 to 0.98), and AUC₀₋ₜ and AUC_{0-inf} (both with an estimate of 1.04 and 90% CI ranging from 0.98 to 1.10).

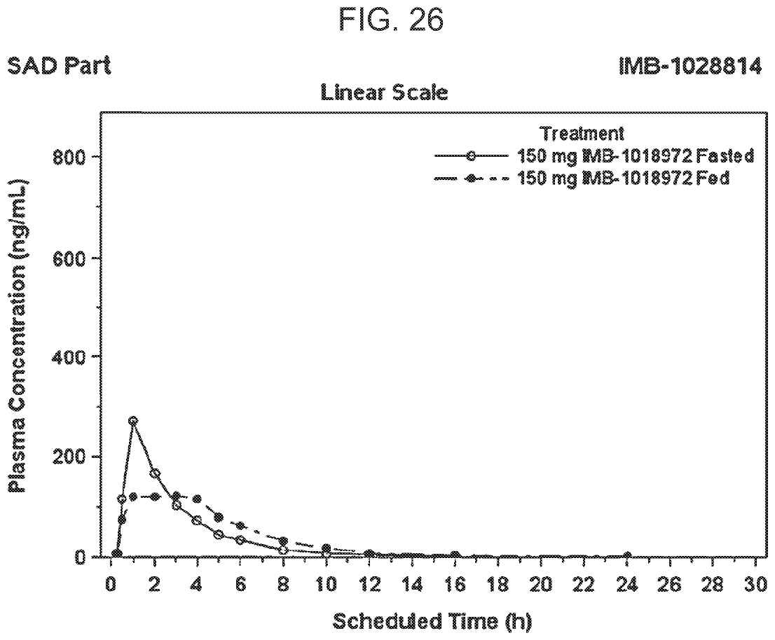

FIG. 26 is a graph of Geometric Mean IMB-1028814 Plasma Concentration-Time Profiles (Linear)—FE Arm of SAD Part (PK Set)

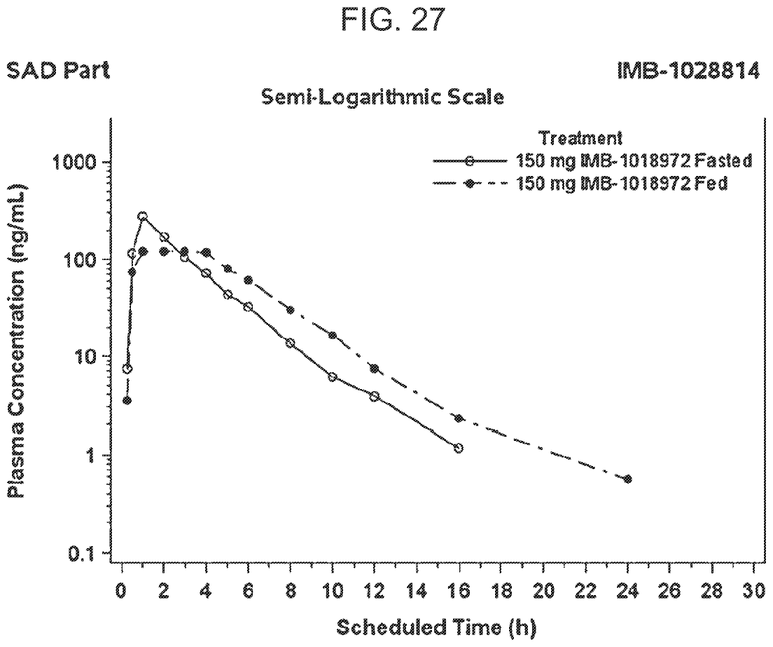

FIG. 27 is a graph of Geometric Mean IMB-1028814 Plasma Concentration-Time Profiles (Semi-Logarithmic Scale)—FE Arm of SAD Part (PK Set)

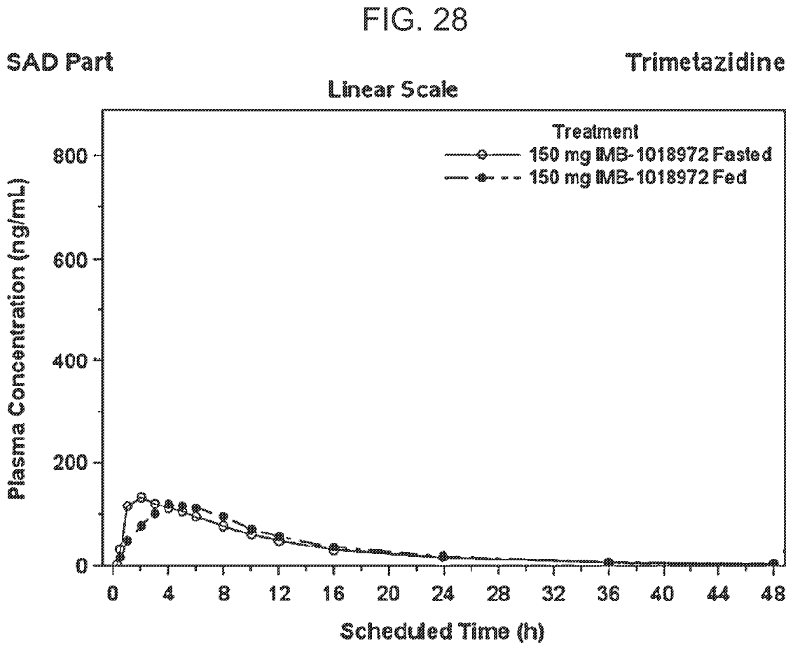

FIG. 28 is a graph of Geometric Mean Trimetazidine Plasma Concentration-Time Profiles (Linear)—FE Arm of SAD Part (PK Set)

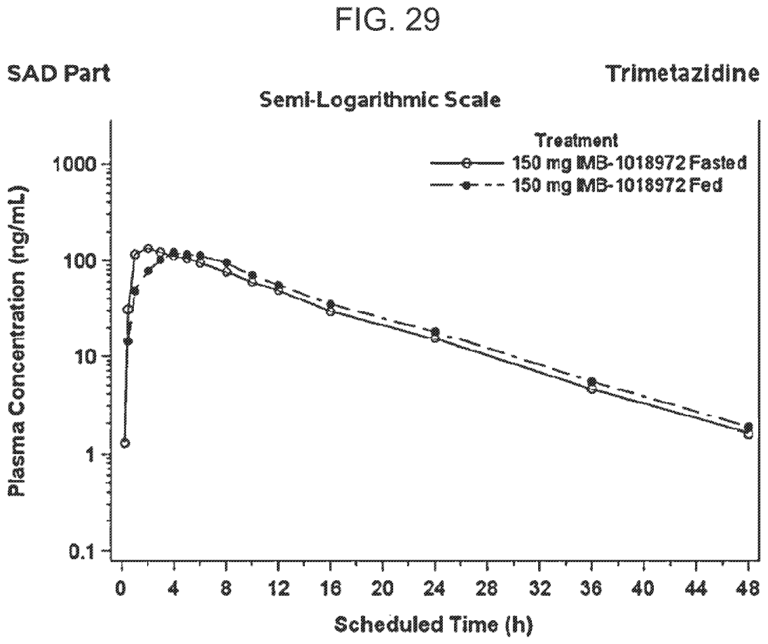

FIG. 29 is a graph of Geometric Mean Trimetazidine Plasma Concentration-Time Profiles (Semi-Logarithmic Scale)—FE Arm of SAD Part (PK Set)

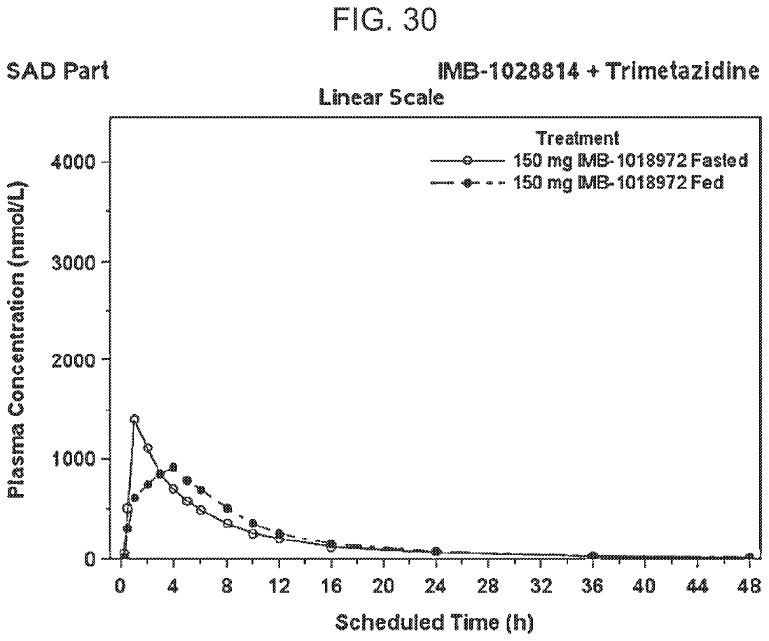

FIG. 30 is a graph of Geometric Mean IMB-1028814+Trimetazidine Plasma Concentration-Time Profiles (Linear)—FE Arm of SAD Part (PK Set)

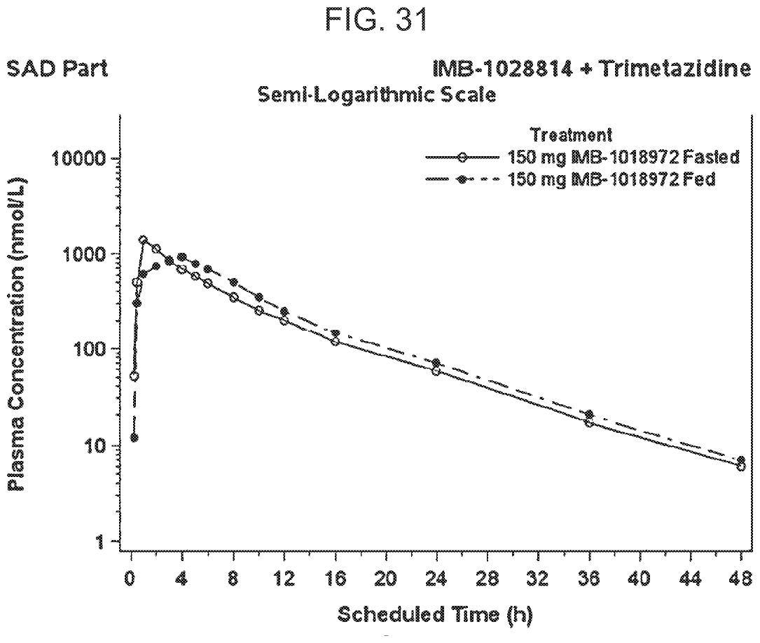

FIG. 31 is a graph of Geometric Mean IMB-1028814+Trimetazidine Plasma Concentration-Time Profiles (Semi-Logarithmic Scale)—FE Arm of SAD Part (PK Set)

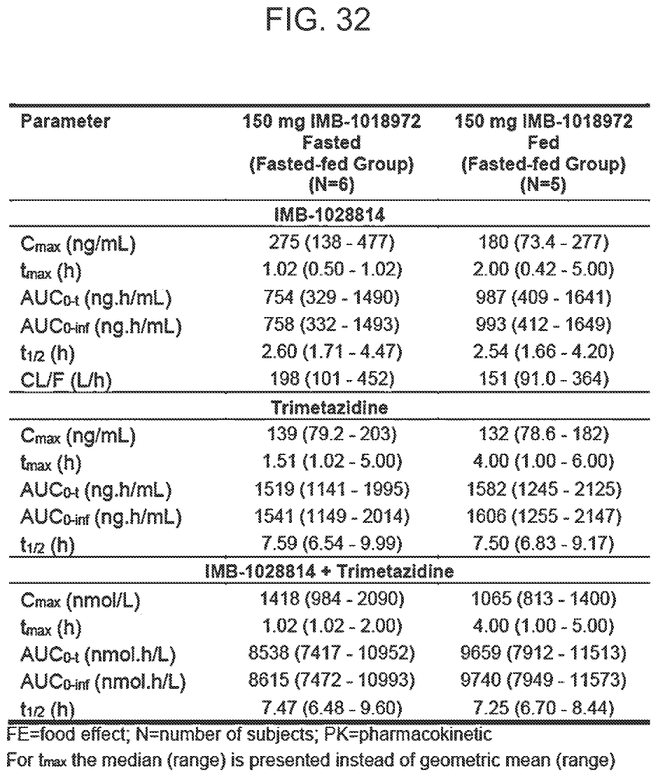

FIG. 32 is a table of Summary Statistics (Geometric Mean [Range]) of IMB-1028814, Trimetazidine, and IMB-128814+Trimetazidine, and IMB-1028814+Trimetazidine Plasma Pharmacokinetic Parameters-FE Arm of SAD Part (PK Set)

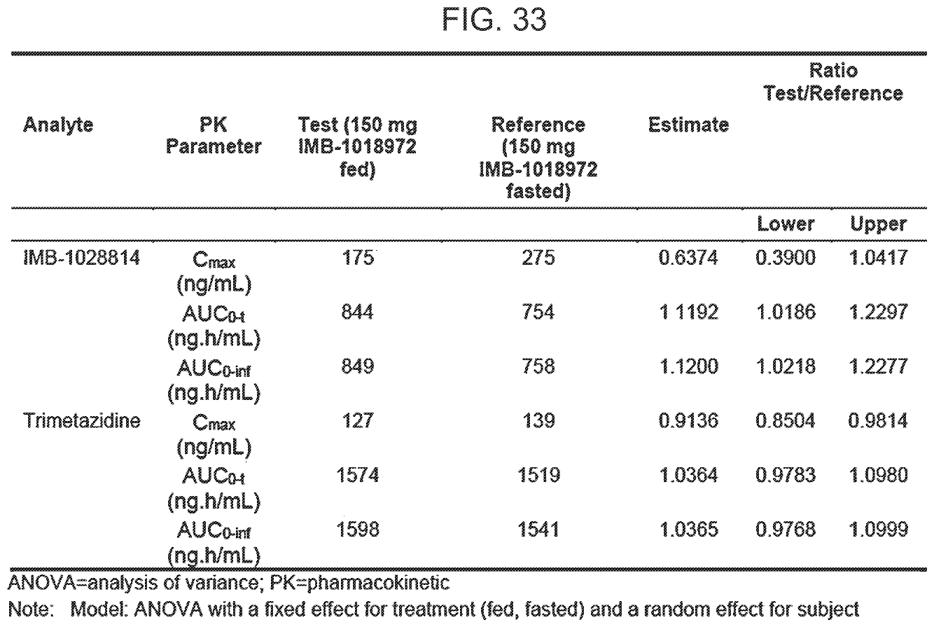

FIG. 33 is a table of Exploratory Analysis of Food Effect for IMB-1028814 and Trimetazidine following Administration of 150 mg IMB-1018972—FE Arm of SAD Part (PK Set)

### Pharmacokinetic Results of IMB-1028814 and Trimetazidine in Urine

Urinary excretion of IMB-1028814 and trimetazidine was determined in urine samples from subjects who received a single oral dose of IMB-1018972 in the range of 50 mg to 400 mg under fasted conditions. Further, urinary excretion of trimetazidine was determined in urine samples from subjects who received a single oral dose of 35 mg trimetazidine.

The arithmetic mean percent of the dose excreted in urine ranged between 3.99% and 5.74% for IMB-1028814, and between 23.11% and 32.55% for trimetazidine within 48 hours after a single oral IMB-1018972 dose over the studied dose range of 50 mg to 400 mg. Within 48 hours following administration of a single oral dose of 35 mg trimetazidine, an arithmetic mean of 54.47% was excreted in urine as trimetazidine. These results indicate that metabolism is the primary clearance mechanism for IMB-1028814 while renal excretion is the primary clearance mechanism for trimetazidine.

The geometric mean renal clearance (CLR) ranged between 3.76 L/h and 5.37 L/h for IMB-1028814, and between 18.1 L/h and 20.8 L/h for trimetazidine over the studied single-dose range of 50 mg to 400 mg IMB-1018972. Geometric mean CLR for trimetazidine was 20.4 L/h following administration of a single oral dose of 35 mg trimetazidine. The renal clearance of trimetazidine is greater than glomerular filtration rate (125 mL/min or 7.5 L/h), indicating that trimetazidine undergoes net tubular secretion.

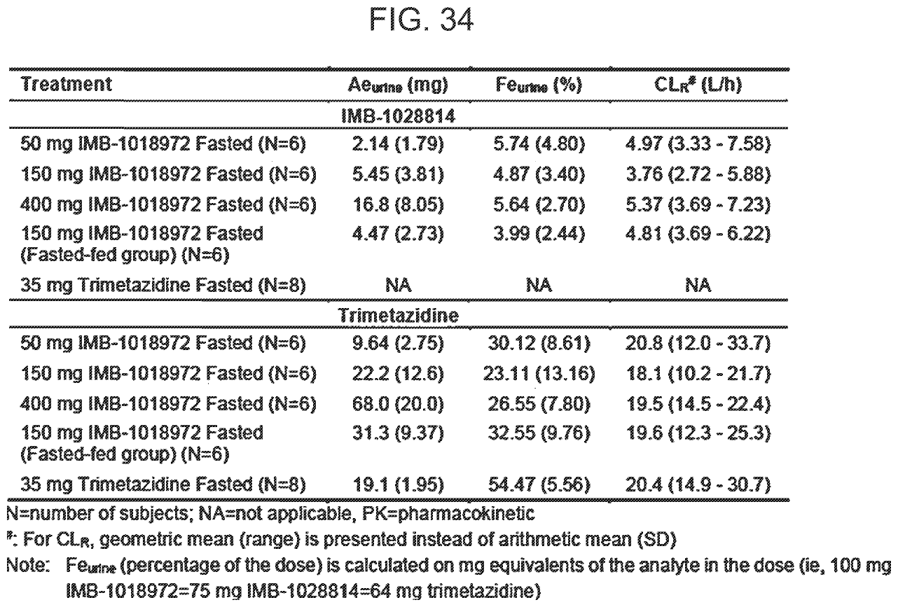

FIG. 34 is a table of Summary Statistics (Arithmetic Mean [SD]) of Urine Pharmacokinetic Parameters for IMB-1028814, Trimetazidine, and IMB-1028814+Trimetazidine—SAD Part (PK Set)

### Pharmacokinetic Results of IMB-1028814 and Trimetazidine in Urine

Urinary excretion of IMB-1028814 and trimetazidine was determined in urine samples from subjects who received a single oral dose of IMB-1018972 in the range of 50 mg to 400 mg under fasted conditions. Further, urinary excretion of trimetazidine was determined in urine samples from subjects who received a single oral dose of 35 mg trimetazidine.

The arithmetic mean percent of the dose excreted in urine ranged between 3.99% and 5.74% for IMB-1028814, and between 23.11% and 32.55% for trimetazidine within 48 hours after a single oral IMB-1018972 dose over the studied dose range of 50 mg to 400 mg. Within 48 hours following administration of a single oral dose of 35 mg trimetazidine, an arithmetic mean of 54.47% was excreted in urine as trimetazidine. These results indicate that metabolism is the primary clearance mechanism for IMB-1028814 while renal excretion is the primary clearance mechanism for trimetazidine.

The geometric mean renal clearance (CLR) ranged between 3.76 L/h and 5.37 L/h for IMB-1028814, and between 18.1 L/h and 20.8 L/h for trimetazidine over the studied single-dose range of 50 mg to 400 mg IMB-1018972. Geometric mean CLR for trimetazidine was 20.4 L/h following administration of a single oral dose of 35 mg trimetazidine. The renal clearance of trimetazidine is greater than glomerular filtration rate (125 mL/min or 7.5 L/h), indicating that trimetazidine undergoes net tubular secretion.

### MAD Part

### Pharmacokinetic Results of IMB-1028814, Trimetazidine and IMB-1028814+Trimetazidine in Plasma

All predose samples on Day 1 were below the LLOQ for IMB-1028814 and trimetazidine plasma concentrations.

The geometric mean concentration-time profiles for IMB-1028814, metabolite trimetazidine, and IMB-1028814+trimetazidine on Day 1 and Day 14 showed a dose dependent increase in plasma concentrations following administration of multiple doses of IMB-1018972 under fed conditions of 50 mg q12 h and 150 mg q12 h.

Similar to the SAD part, initial hydrolysis of IMB-1018972 to IMB-1028814 and subsequent systemic bioavailability of IMB-1028814 on Days 1 and 14 was relatively rapid. Over the 2 multiple-dose levels, median tmax ranged between 0.5 hours and 1 hours postdose for IMB-1028814 on Day 1, and was 3 hours postdose for trimetazidine on Day 1. On Day 14, median tmax was 0.5 hours postdose for IMB-1028814 and 2 hours postdose for trimetazidine.

### Exposure Parameters on Day 1

No dose-proportionality analysis was done since there were only 2 IMB-1018972 dose levels in the MAD part: multiple oral doses of 50 mg or 150 mg q12 h for 14 days under fed conditions.

The geometric mean Cₘₐₓ and AUC_{0-T} were higher after 150 mg fed than after 50 mg fed for IMB-1028814 (297% and 336% higher for Cₘₐₓ and AUC_{0-T}, respectively), trimetazidine (154% and 163% higher for Cₘₐₓ and AUC_{0-T}, respectively), and IMB-1028814+trimetazidine (257% and 239% higher for Cₘₐₓ and AUC_{0-T}, respectively).

When comparing the MAD and SAD parts, geometric mean Cₘₐₓ was 97% higher on Day 1 after 150 mg fed in the MAD part than after a single dose of 150 mg fed in the SAD part for IMB-1028814. For trimetazidine however, geometric mean Cₘₐₓ was 32% lower on Day 1 after 150 mg fed in the MAD part than after a single dose of 150 mg fed in the SAD part.

### Exposure Parameters on Day 14 Following Repeated q12 h Dosing

The geometric mean Cₘₐₓ and AUC_{0-T} were higher after 150 mg fed than after 50 mg fed for IMB-1028814 (377% and 367% higher for Cₘₐₓ and AUC_{0-T}, respectively), trimetazidine (127% and 126% higher for Cₘₐₓ and AUC_{0-T}, respectively), and IMB-1028814+trimetazidine (286% and 211% higher for Cₘₐₓ and AUC_{0-T}, respectively).

The predefined stopping criterion for IMB-1028814 plasma exposure of 417,733 and 652,849 ng·h/mL for males and females, respectively, was not reached by any of the subjects during the MAD part.

### Trough Concentrations Following Repeated q12 h Dosing

Based upon visual inspection of the geometric mean plasma concentration-time profiles and the geometric mean trough concentrations, it can be concluded that for both 150 mg fed and 50 mg fed, the Day 14 IMB-1018972 dose was administered under steady-state conditions of IMB-1028814 and trimetazidine concentrations

### Accumulation Following Repeated q12 h Dosing

For both the 50 mg and 150 mg fed dose levels, geometric mean AUC_{0-T} values of IMB-1028814, trimetazidine, and IMB-1028814+trimetazidine were higher on Day 14 relative to Day 1.

Geometric mean R_{ac} for IMB-1028814 was 1.18 and 1.10 after the 150 mg fed dose and 50 mg fed dose, respectively, indicating minimal accumulation of IMB-1028814 in plasma. Geometric mean R_{ac} for trimetazidine was 1.63 and 1.89 after the 150 mg fed dose and 50 mg fed dose, respectively, indicating modest accumulation of trimetazidine in plasma. Geometric mean R_{ac} for IMB-1028814+trimetazidine was 1.39 and 1.52 after the 150 mg fed dose and 50 mg fed dose, respectively, indicating modest accumulation of IMB-1028814+trimetazidine in plasma.

### Terminal Elimination Half-Life Following Repeated q12 h Dosing

For IMB-1028814, the geometric mean t1/2 of 4.48 hours after 150 mg fed was longer than that of 2.79 hours after 50 mg fed. For trimetazidine, the geometric mean t1/2 of 9.36 hours after 150 mg fed was similar to that of 9.32 hours after 50 mg fed. For IMB-1028814+trimetazidine, the geometric mean t1/2 of 8.90 hours for IMB-1028814 after 150 mg fed was similar to that of 9.08 hours after 50 mg fed.

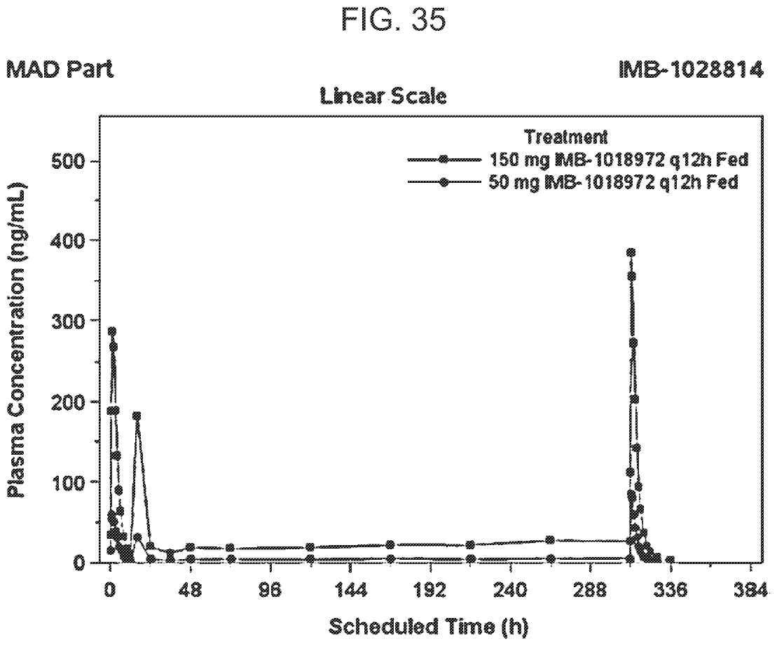

FIG. 35 is a graph of Geometric Mean IMB-1028814 Plasma Concentration-Time Profiles from Day 1 through Day 14 (Linear)—MAD Part (PK Set)

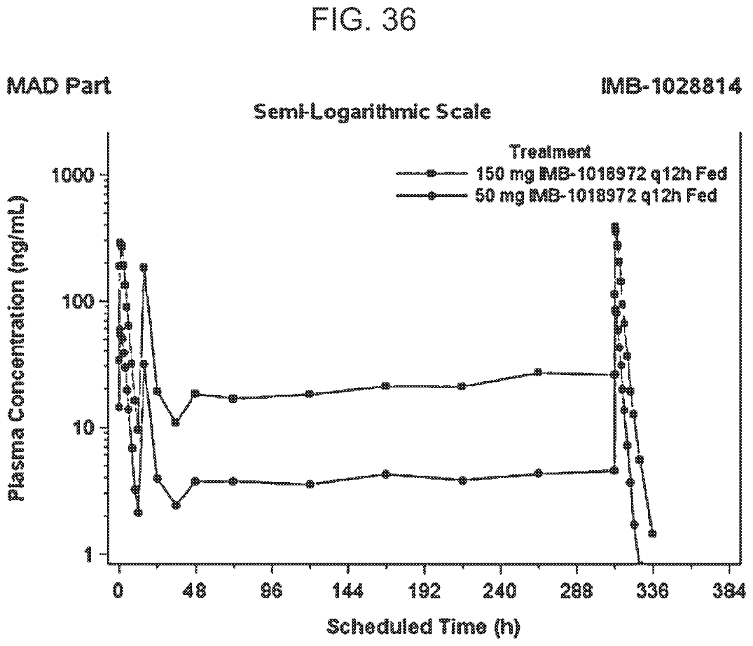

FIG. 36 is a graph of Geometric Mean IMB-1028814 Plasma Concentration-Time Profiles from Day 1 through Day 14 (Semi-Logarithmic Scale)—MAD Part (PK Set)

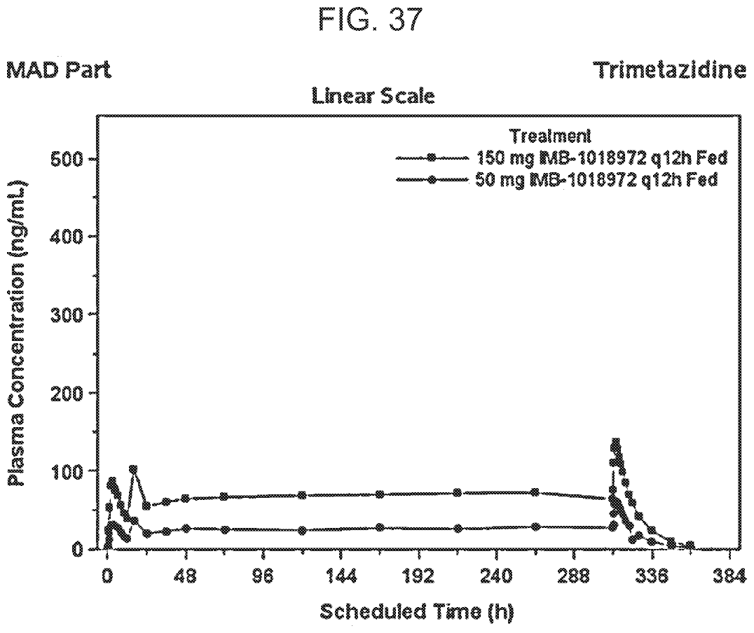

FIG. 37 is a graph of Geometric Mean Trimetazidine Plasma Concentration-Time

Profiles from Day 1 through Day 14 (Linear)—MAD Part (PK Set)

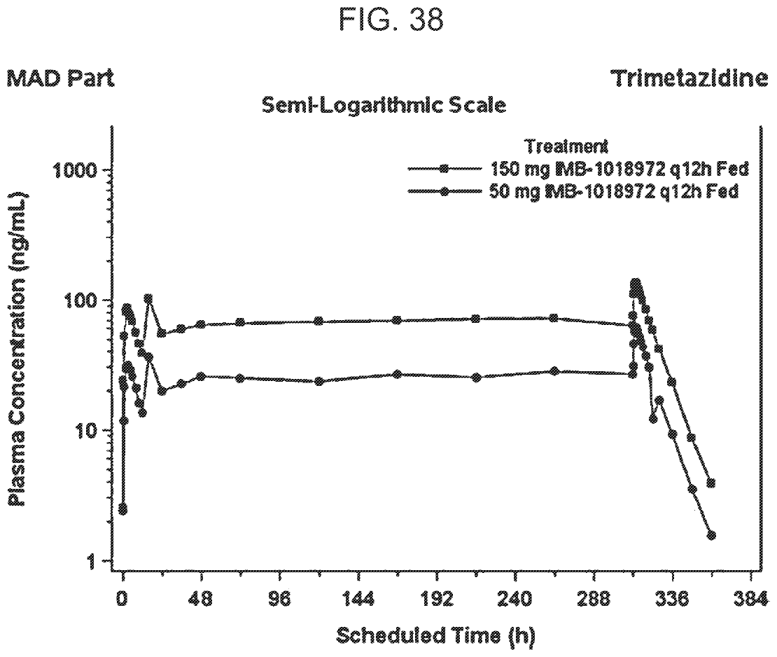

FIG. 38 is a graph of Geometric Mean Trimetazidine Plasma Concentration-Time Profiles from Day 1 through Day 14 (Semi-Logarithmic Scale)—MAD Part (PK Set)

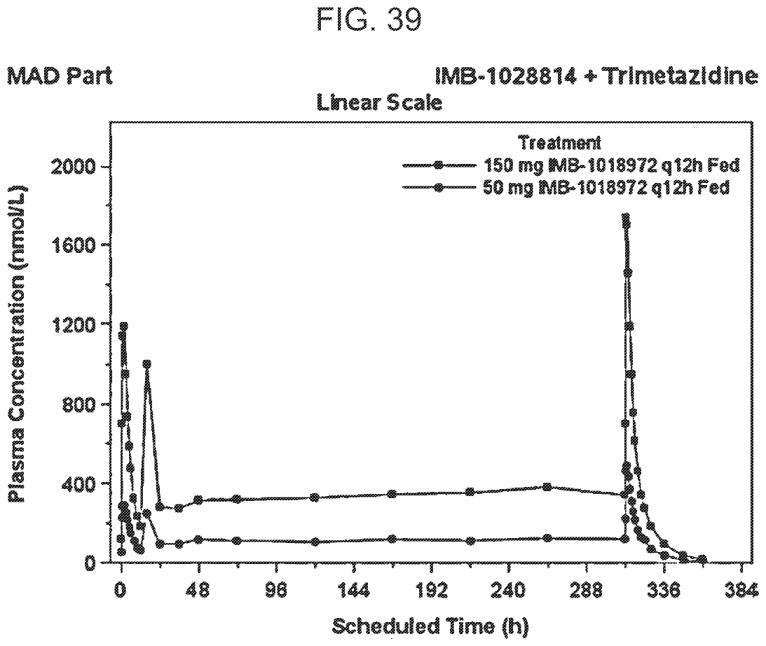

FIG. 39 is a graph of Geometric Mean IMB-1028814+Trimetazidine Plasma Concentration-Time Profiles from Day 1 through Day 14 (Linear)—MAD Part (PK Set)

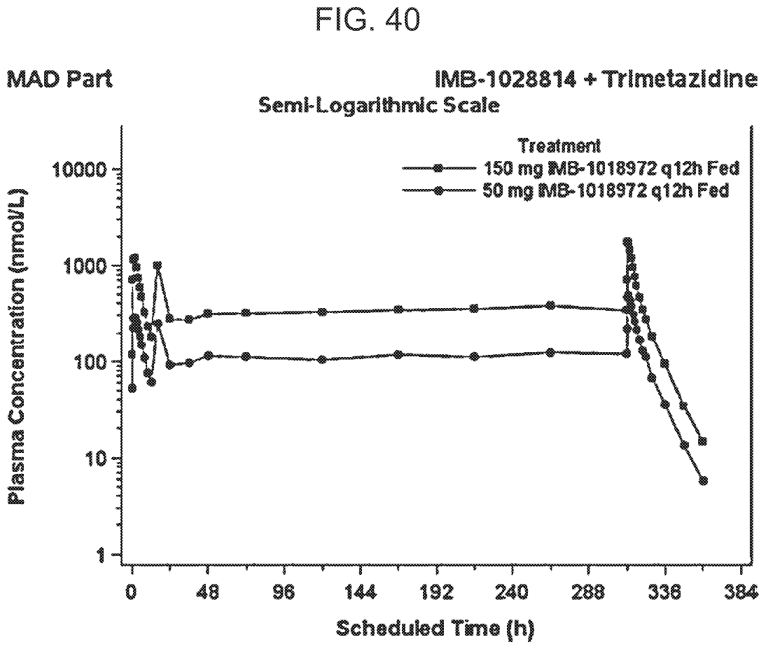

FIG. 40 is a graph of Geometric Mean IMB-1028814+Trimetazidine Plasma Concentration-Time Profiles from Day 1 through Day 14 (Semi-Logarithmic Scale)—MAD Part (PK Set)

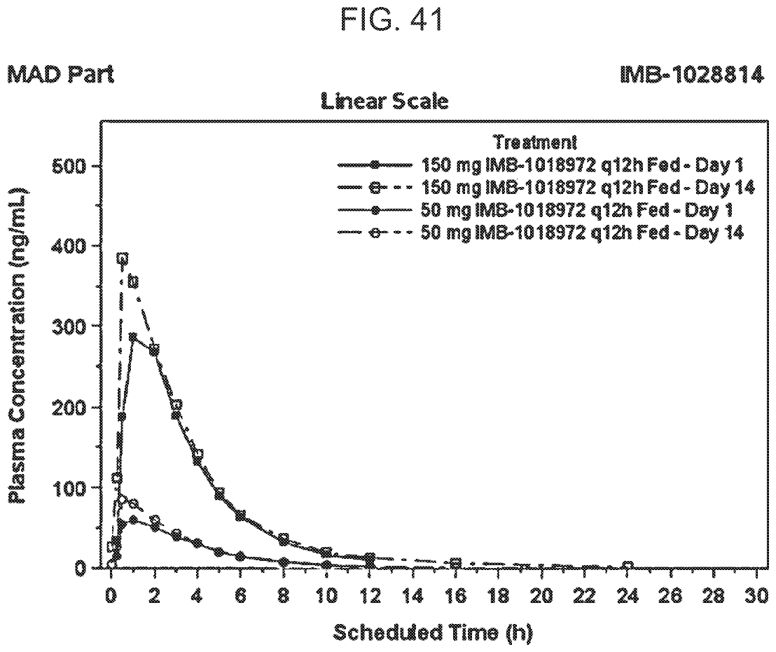

FIG. 41 is a graph of Geometric Mean IMB-1028814 Plasma Concentration-Time Profiles from Day 1 through Day 14 (Linear)—MAD Part (PK Set)

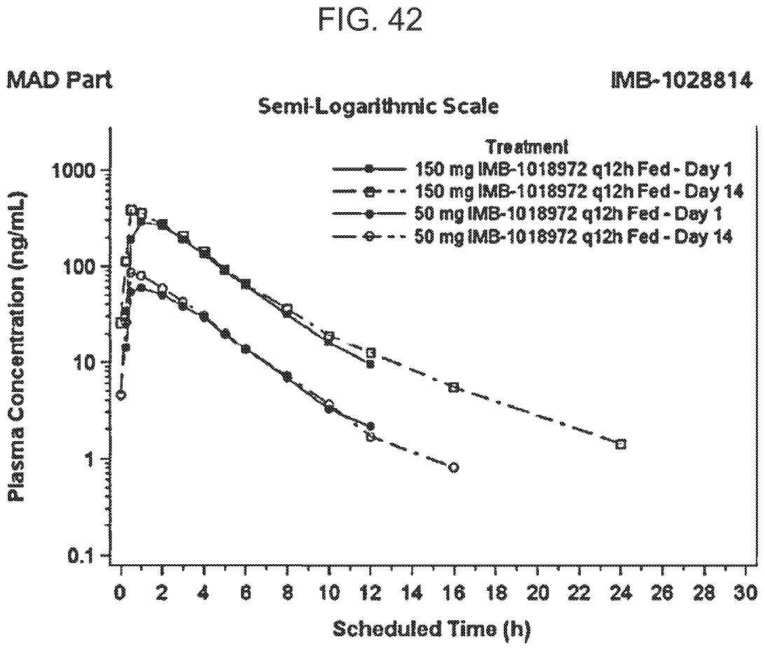

FIG. 42 is a graph of Geometric Mean IMB-1028814 Plasma Concentration-Time Profiles from Day 1 through Day 14 (Semi-Logarithmic Scale)—MAD Part (PK Set)

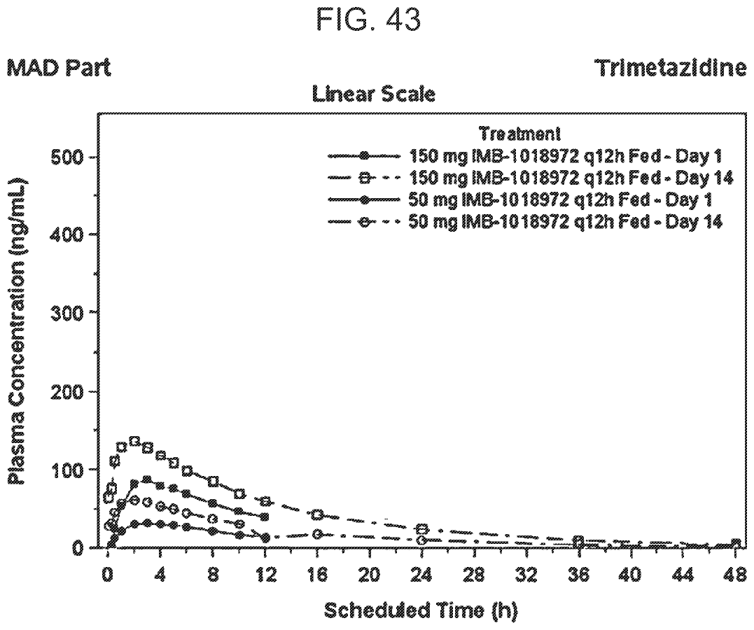

FIG. 43 is a graph of Geometric Mean Trimetazidine Plasma Concentration-Time Profiles after Dosing on Day 1 through Day 14 (Linear)—MAD Part (PK Set)

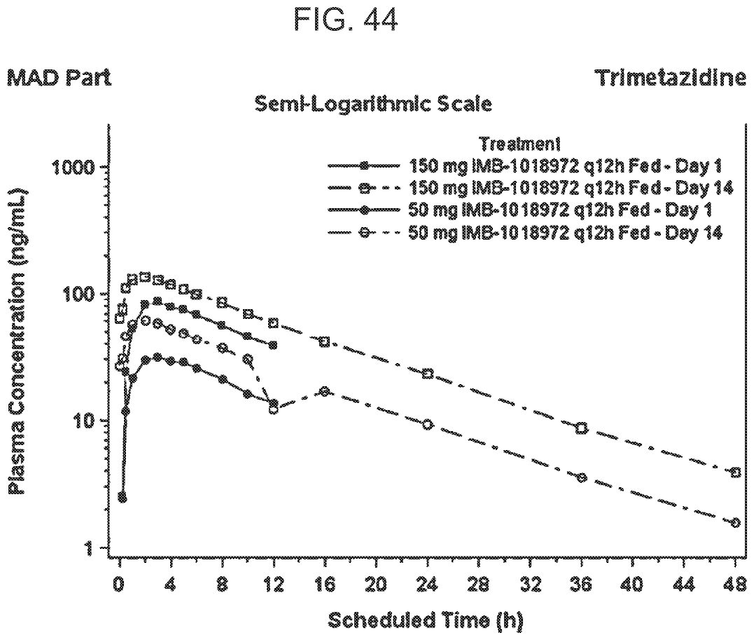

FIG. 44 is a graph of Geometric Mean Trimetazidine Plasma Concentration-Time Profiles after Dosing on Day 1 through Day 14 (Semi-Logarithmic Scale)—MAD Part (PK Set)

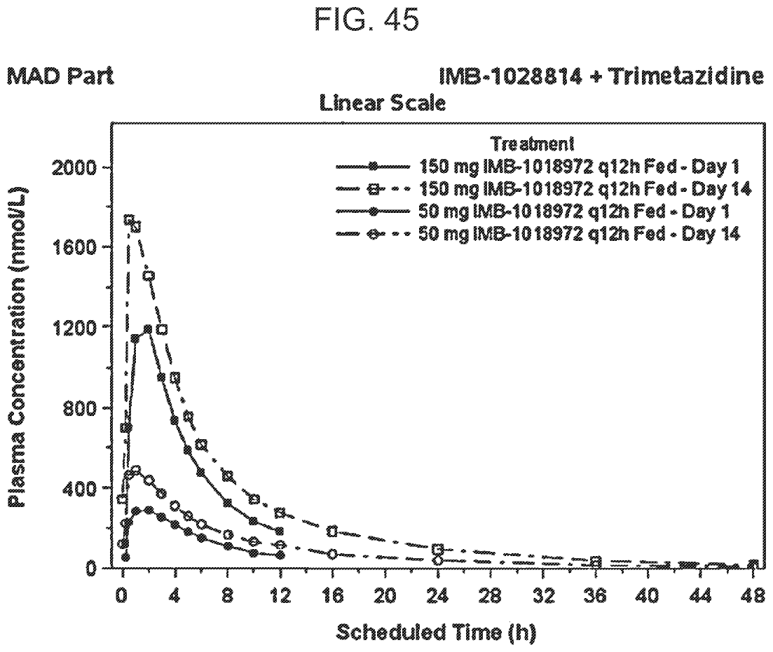

FIG. 45 is a graph of Geometric Mean IMB-1028814+Trimetazidine Plasma Concentration-Time Profiles after Dosing on Day 1 through Day 14 (Linear)—MAD Part (PK Set)

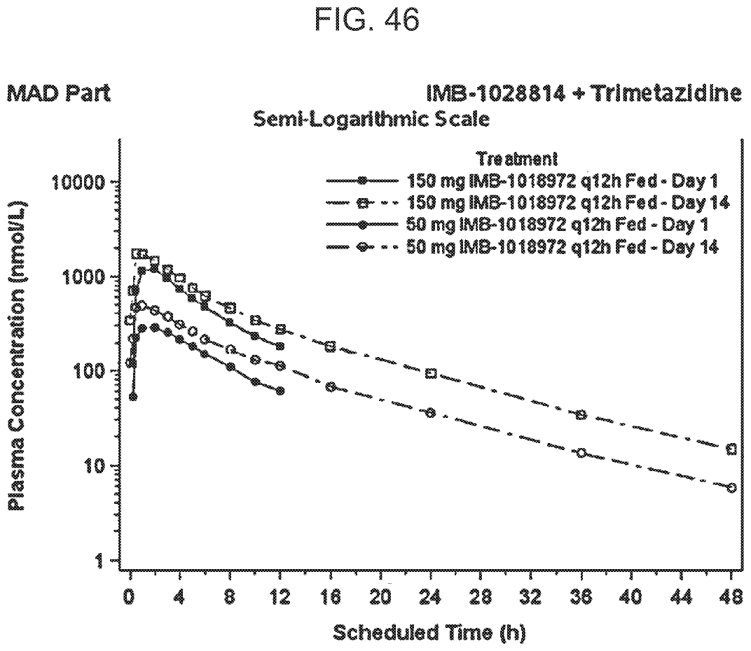

FIG. 46 is a graph of Geometric Mean IMB-1028814+Trimetazidine Plasma Concentration-Time Profiles after Dosing on Day 1 through Day 14 (Semi-Logarithmic Scale)—MAD Part (PK Set)

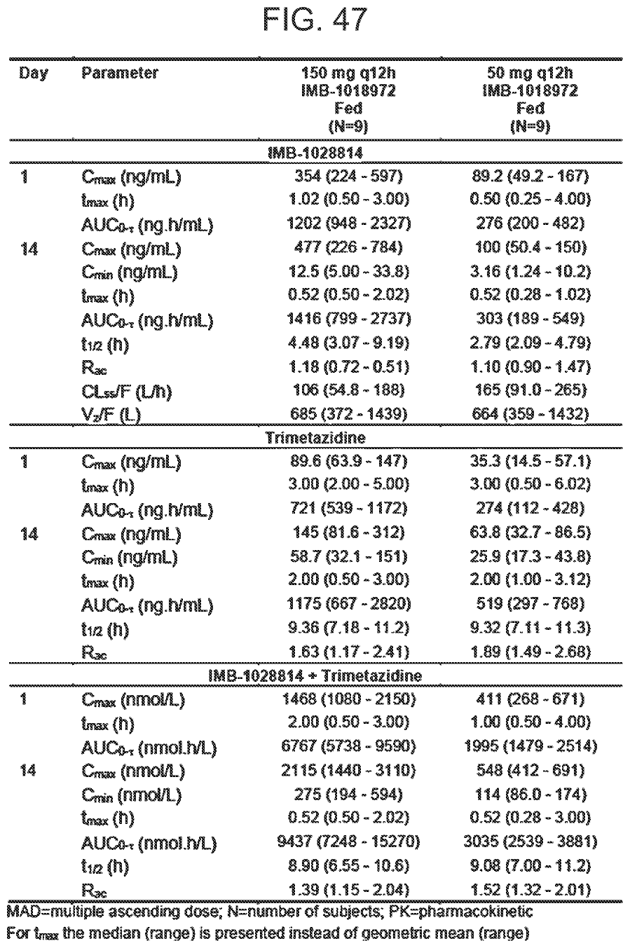

FIG. 47 is a table of Summary Statistics (Geometric Mean [Range]) of IMB-1028814, Trimetazidine, and IMB-1028814+Trimetazidine Plasma Pharmacokinetic Parameters—MAD Part (PK Set)

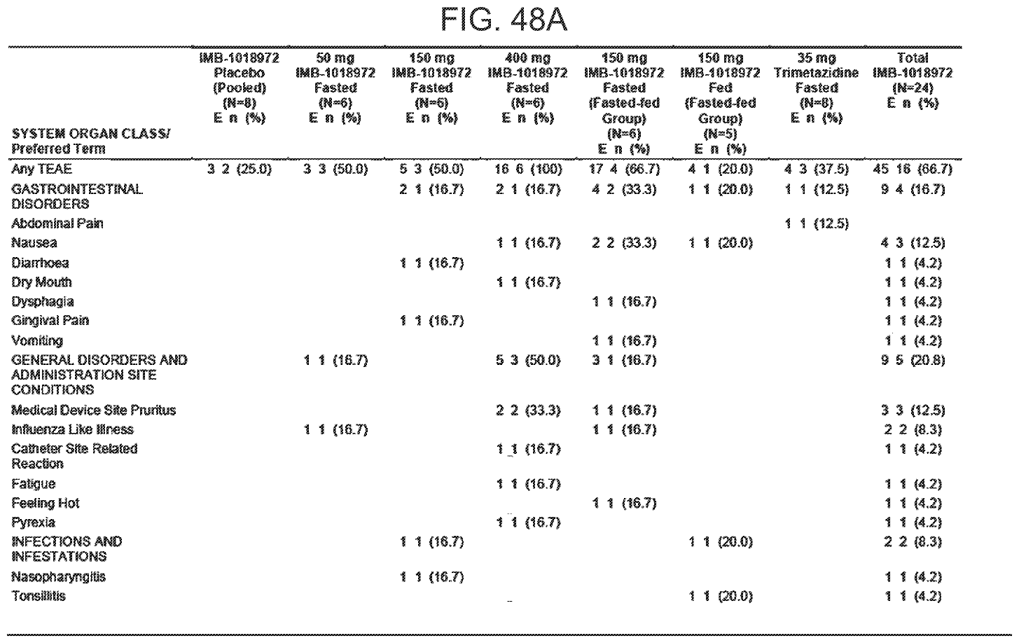

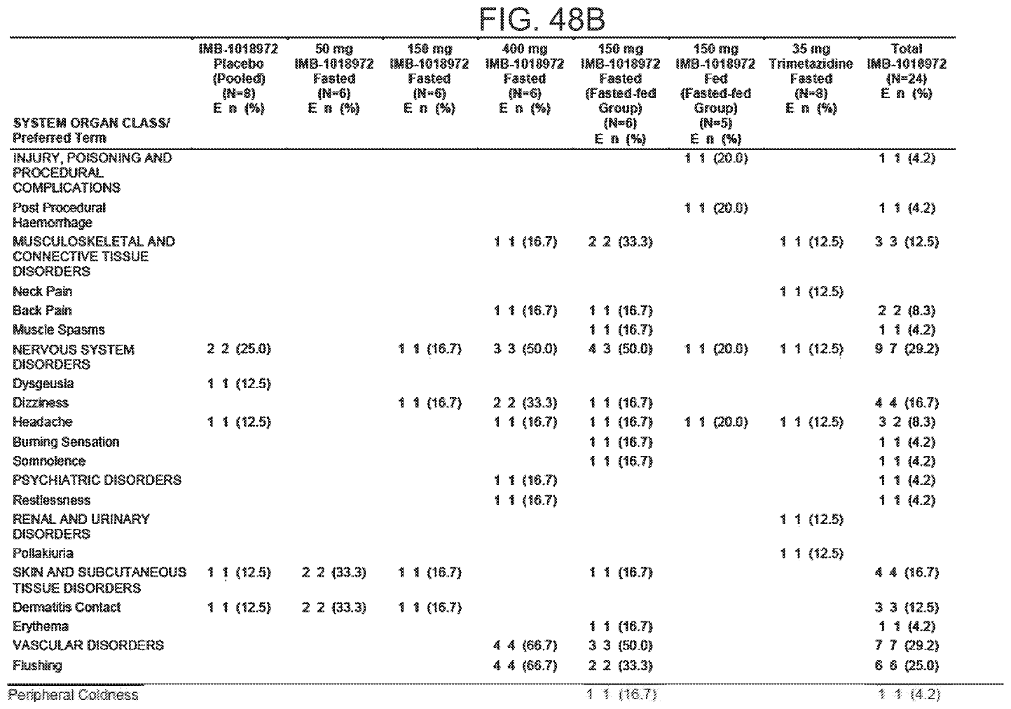

FIG. 48A and FIG. 48B is a table Summary of All TEAEs by System Organ Class, Preferred Term and Treatment—SAD Part (and integrated FE Arm) (Safety Set) with the following notifications:

%=number of subjects (n) as a percentage of number of subjects (N) per treatment; AE=adverse event; E=number of AEs; FE=food effect; MedDRA=Medical Dictionary for Regulatory Activities; N=number of subjects exposed; n=number of subjects that experienced the AE; SAD=single ascending dose; TEAE-treatment-emergent adverse event

Adverse events were classified according to MedDRA 22.0

Subjects were counted once, per preferred term, for multiple occurrences of a specific MedDRA term

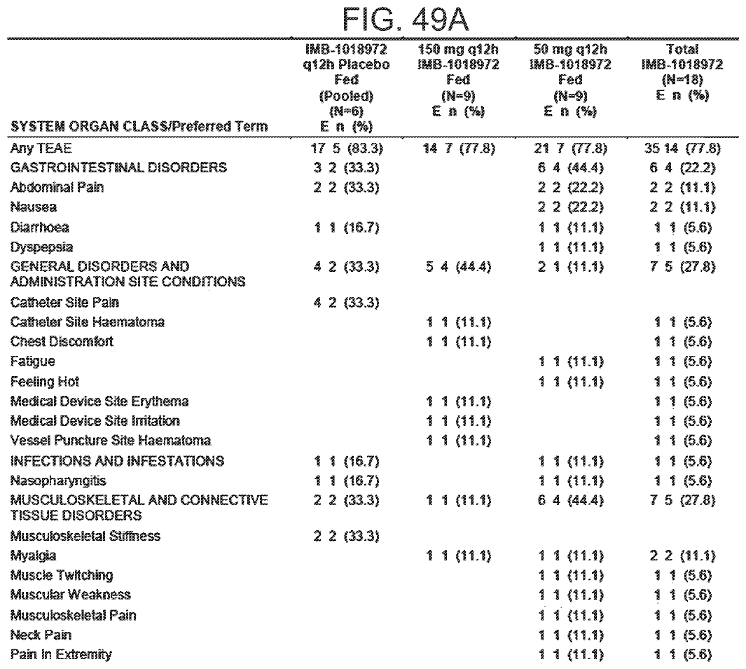

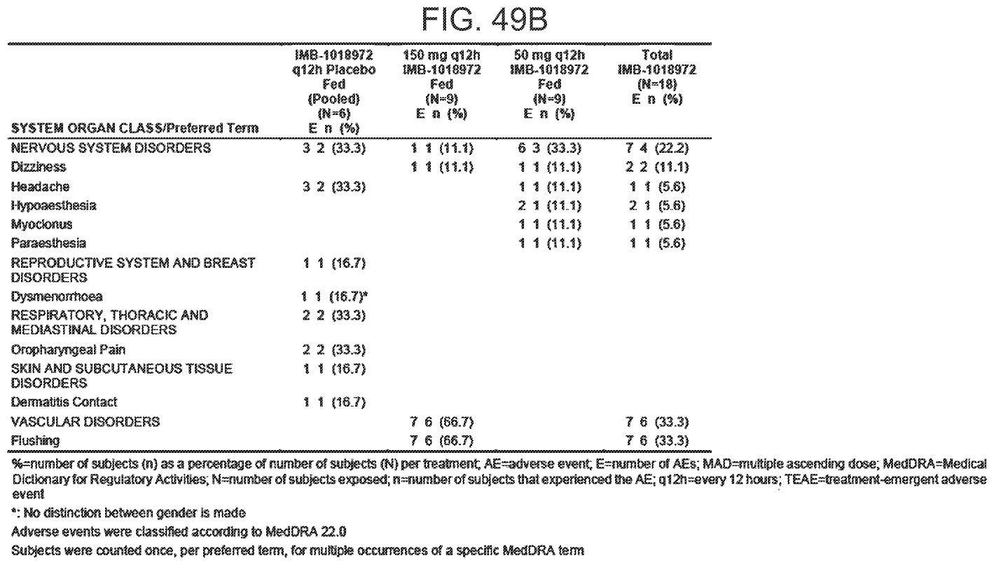

FIG. 49A and FIG. 49B is a table Summary of All TEAEs by System Organ Class, Preferred Term and Treatment—MAD Part (Safety Set)

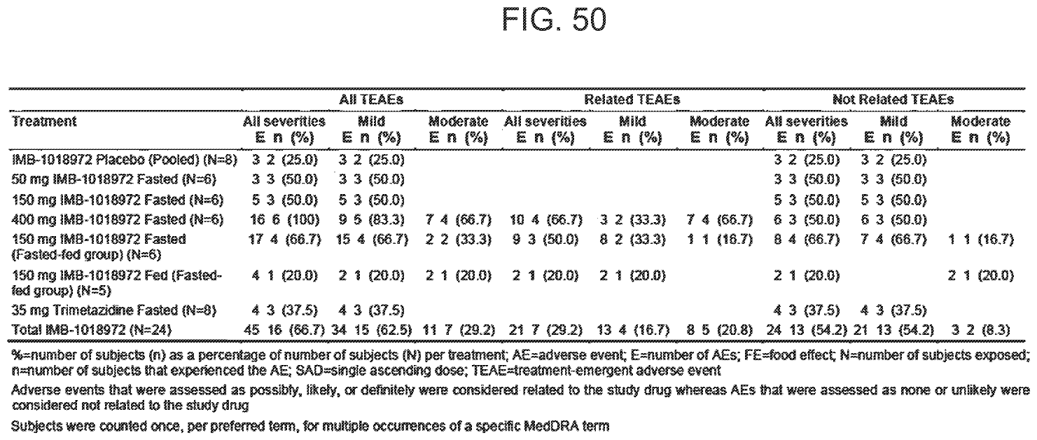

FIG. 50 is a table Summary of All TEAEs by Treatment, Relationship, and Severity—SAD Part (and Integrated FE Arm) (Safety Set)

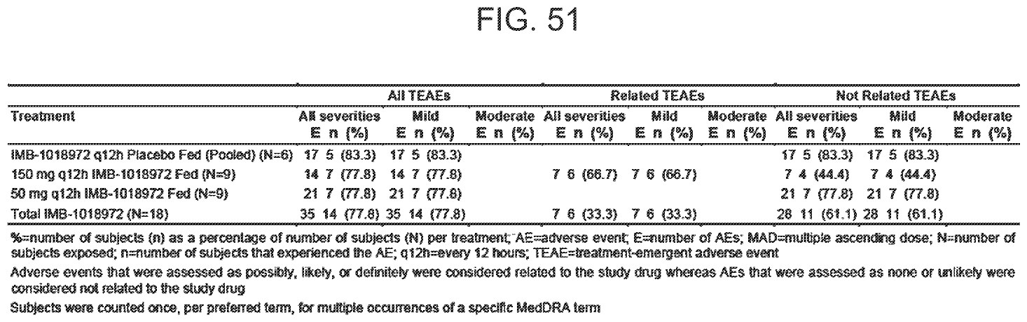

FIG. 51 is a table Summary of All TEAEs by Treatment, Relationship, and Severity—MAD Part (Safety Set)

### Summary of Adverse Events

### SAD Part (and Integrated FE Arm)

TEAEs Reported with Administration of IMB-1018972 or Placebo

A total of 45 TEAEs was reported by 16 of 24 (66.7%) subjects who received IMB-1018972, and a total of 3 TEAEs was reported by 2 of 8 (25%) subjects who received placebo. There were no deaths reported and all TEAEs were transient and resolved without sequelae by follow-up. Subject 131 of the FE arm Group A4 was withdrawn from the study due to a moderate SAE of influenza like illness (unlikely related) in the first period after receiving the single oral dose of 150 mg IMB-1018972 under fasted conditions.

Thirty-seven of 48 TEAEs reported with IMB-1018972 or placebo were of mild severity and 11 TEAEs were of moderate severity. No severe TEAEs were reported. The 11 moderate TEAEs were as follows:
Five moderate TEAEs of flushing (reported term was ‘niacin flush’) were reported by 5 subjects (Subjects 117, 122, 123, 124, and 132). Subjects 117, 122, 123, and 124 reported flushing after a single dose of 400 mg IMB-1018972 under fasted conditions. Subject 132 of the FE arm reported flushing after a single dose of 150 mg IMB-1018972 under fasted conditions. These TEAEs were all considered by the Investigator to be definitely related to the study drug. The niacin flushing events observed in this study were typically short-lasting with generalized cutaneous vasodilation and to varying degrees associated with an intense burning and tingling sensation of the skin, a feeling of warmth, and/or generalized erythema, starting shortly after intake of the drug and lasting about 1 to 2.5 hours.
One moderate TEAE each of restlessness, back pain, and nausea was reported by 1 subject (Subject 122) who also reported a moderate TEAE of flushing. This subject had received a single dose of 400 mg IMB-1018972 under fasted conditions. The TEAEs of back pain and nausea were considered by the Investigator to be possibly related to the study drug, whereas the Investigator considered the TEAE of restlessness to be likely related.
One moderate TEAE each of tonsillitis and post procedural hemorrhage (reported term was ‘post tonsillectomy hemorrhage’) was reported by 1 subject (Subject 129). This subject had received a single dose of 150 mg IMB-1018972 under fed conditions. The TEAEs of tonsillitis and post procedural hemorrhage were considered by the Investigator not to be related to the study drug.
One TEAE of influenza like illness of moderate severity was reported by 1 subject (Subject 131) and was considered by the Investigator to be an SAE and unlikely related to the study drug. This subject had received a single dose of 150 mg IMB-1018972 under fasted conditions and did not receive the planned dose in the fed state.

Of 48 TEAEs, 3 were reported by 2 (25.0%) subjects receiving placebo, 3 were reported by 3 (50.0%) subjects receiving 50 mg IMB-1018972 under fasted conditions, 5 were reported by 3 (50.0%) subjects receiving 150 mg IMB-1018972 under fasted conditions, 16 were reported by 6 (100%) subjects receiving 400 mg IMB-1018972 under fasted conditions, 17 were reported by 4 (66.7%) subjects receiving 150 mg IMB-1018972 under fasted conditions (in the fasted-fed group), and 4 were reported by 1 (20%) subject receiving 150 mg IMB-1018972 under fed conditions (in the fasted-fed group). There was no clear dose dependency of the number and incidence of TEAEs. Neither was there any clear difference between fasted and fed IMB-1018972 administration for the number and incidence of TEAEs.

The most frequently reported TEAEs (ie, reported by ≥15% of the subjects) with IMB-1018972 by system organ class (SOC) were:
Nervous system disorders with 9 TEAEs reported by 7 (29.2%) subjects (4 TEAEs of dizziness, 3 TEAEs of headache, and 1 TEAE each of burning sensation and somnolence).
Vascular disorders with 7 TEAEs reported by 7 (29.2%) subjects (6 TEAEs of flushing and 1 TEAE of peripheral coldness).
General disorders and administration site conditions with 9 TEAEs reported by 5 (20.8%) subjects (3 TEAEs of medical device site pruritus, 2 TEAEs of influenza like illness, and 1 TEAE each of catheter site related reaction, fatigue, feeling hot, and pyrexia).
Gastrointestinal disorders with 9 TEAEs reported by 4 (16.7%) subjects (4 TEAEs of nausea and 1 TEAE each of diarrhea, dry mouth, dysphagia, gingival pain, and vomiting).
Skin and subcutaneous tissue disorders with 4 TEAEs reported by 4 (16.7%) subjects (3 TEAEs of dermatitis contact and 1 TEAE of erythema).

Of 45 TEAEs reported with IMB-1018972, 21 TEAEs reported by 7 of 24 (29.2%) subjects were considered by the Investigator to be related to the study drug. No drug-related TEAEs were reported following 50 mg and 150 mg (fasted only group) IMB-1018972. The most frequently reported drug-related TEAEs (ie, reported by ≥15% of the subjects) with IMB-1018972 by SOC were:
Vascular disorders with 6 TEAEs of flushing reported by 6 (25%) subjects.
Nervous system disorders with 5 TEAEs reported by 4 (16.7%) subjects (3 TEAEs of headache and 2 TEAEs of dizziness).
   TEAEs Reported with Administration of Trimetazidine

A total of 4 TEAEs was reported by 3 of 8 (37.5%) subjects who received trimetazidine. There were no deaths reported and all TEAEs were transient and resolved without sequelae by follow-up. All 4 TEAEs (1 TEAE each of neck pain, abdominal pain, pollakiuria, and headache) reported were of mild severity and considered by the Investigator not to be related to the study drug.

### Overall Tolerability

In the SAD part, treatment with single oral doses of 50 mg, 150 mg, and 400 mg IMB-1018972 under fasted conditions, treatment with single oral doses of 150 mg IMB-1018972 under fed conditions, and treatment with single oral doses of 35 mg trimetazidine were well tolerated by healthy male and female subjects. In the FE arm of the SAD part, dosing under fed conditions appeared to attenuate the number and incidence of TEAEs. During the SAD part, the most common AEs were 6 TEAEs of flushing (reported terms were ‘niacin flush’ and ‘flushing neck’), of which 5 TEAEs were of moderate severity and 1 TEAE was of mild severity. Four subjects reported flushing after a single dose of 400 mg IMB-1018972 under fasted conditions, and 2 subjects of the FE arm reported flushing after a single dose of 150 mg IMB-1018972 under fasted conditions. These TEAEs were all considered by the Investigator to be related to the study drug. No subjects dropped out due to flushing and flushing was not considered a safety issue. There were no clinically important trends in the physical examinations, vital signs, clinical laboratory, or ECG results. Dose escalation beyond 400 mg IMB-1018972 IR did not proceed as planned based on the PK exposure levels of IMB-1028814 and trimetazidine exceeding the target exposure levels in the 400 mg group and the findings of flushing at that dose. The predefined target exposure level was approximately 3 to 4 ‘trimetazidine equivalents’, ie, the ratio of the combined exposure of the active metabolites of IMB1018972 to the single oral doses of 35 mg MR trimetazidine as seen in published literature.

### MAD Part

A total of 35 TEAEs was reported by 14 of 18 (77.8%) subjects who received IMB-1018972, and a total of 17 TEAEs was reported by 5 of 6 (83.3%) subjects who received placebo. All TEAEs were of mild severity and there were no deaths reported. The majority of the TEAEs were transient and resolved without sequelae by follow-up. Three TEAEs were still ongoing at follow-up: vessel puncture site hematoma, medical device site irritation, and paresthesia of the left hand.

Of 35 TEAEs reported by subjects receiving IMB-1018972, 14 were reported by 7 (77.8%) subjects receiving 150 mg IMB-1018972 q12 h under fed conditions, and 21 were reported by 7 (77.8%) subjects receiving 50 mg IMB-1018972 q12 h under fed conditions. There was no clear dose dependency of the number and incidence of TEAEs.

The most frequently reported TEAEs (ie, reported by ≥25% of the subjects) with IMB-1018972 by SOC were:
Vascular disorders with 7 TEAEs of flushing reported by 6 (33.3%) subjects.
General disorders and administration site conditions with 7 TEAEs reported by 5 (27.8%) subjects (1 TEAE each of catheter site hematoma, chest discomfort, fatigue, feeling hot, medical device site erythema, medical device site irritation, and vessel puncture site hematoma).
Musculoskeletal and connective tissue disorders with 7 TEAEs reported by 5 (27.8%) subjects (2 TEAEs of myalgia and 1 TEAE each of muscle twitching, muscular weakness, musculoskeletal pain, neck pain, and pain in extremity).

Of 35 TEAEs reported with IMB-1018972, 7 TEAEs reported by 6 of 18 (33.3%) subjects were considered by the Investigator to be related to the study drug and 28 TEAEs reported by 11 of 18 (61.1%) subjects were considered by the Investigator not to be related to the study drug. All 7 reported drug-related TEAEs were events of flushing and all of these were reported following the highest multiple dose of 150 mg IMB-1018972 q12 h under fed conditions.

### Overall Tolerability

Fourteen-day treatment with oral q12 h doses of 50 mg and 150 mg IMB-1018972 under fed conditions was well tolerated by healthy male and female subjects. Incidental mild TEAEs of flushing occurred in 6 subjects who had received 150 mg IMB-1018972 q12 h. Five of these 6 subjects reported only a single TEAE of flushing during the 14 days dosing period. One subject reported flushing twice, on Day 2 and on Day 14. The severity of flushing was less in the 150 mg IR MAD group relative to that in the 400 mg IR SAD group. No TEAEs of flushing were reported following administration of 50 mg IMB-1018972 q12 h. No subjects dropped out and no modification of the dose was needed due to the TEAEs of flushing.

### Deaths, Other Serious Adverse Events, and Other Significant Adverse Events

One subject was withdrawn during the study.

Subject 131 was a 25-year old white male with a BMI of 21.9 kg/m2. The subject participated in the FE arm Group A4 and was planned to receive 150 mg IMB-1018972 under fasted conditions in the first treatment period and 150 mg IMB-1018972 under fed conditions in the second treatment period. Initially, he reported no relevant medical history and received no concomitant medication at baseline. The subject received a single dose of 150 mg IMB-1018972 under fasted conditions on Day 1 of the first period. Within half an hour after dosing, the subject reported mild short-lasting TEAEs of dizziness, feeling hot, flushing, nausea, and dysphagia, which were all considered by the Investigator to be likely related. He recovered swiftly and completely, and safety assessments including clinical laboratory results showed no abnormalities throughout the in-house period. The subject left the clinic on Day 3 as planned. On Day 5, the subject was assessed by a healthcare provider for the event of flu like symptoms and spontaneous generalized myalgia. On Day 6, the subject was also assessed for the event of anuria despite ample fluid intake. On Day 7, the subject was referred to a hospital where he was immediately hydrated intravenously. Diuresis did not resume immediately and consequently he was admitted to the hospital. The subject's body temperature on admission was 38° C. Hydration was continued and during the evening and night diuresis resumed. The subject's clinical condition improved rapidly, and the subject was discharged on Day 8. Further medical history elucidated dengue fever (December 2018) and viral infection of unknown origin (January 2019) in the months prior to the clinical study and were added to the subject's medical history (this medical history has not been added to the database). A nonspecific diagnosis was established in the hospital. The hospital summarized the event as anuria with normal renal functions, no abnormalities in urinalysis, and resumption of diuresis during admission. The Investigator reported normal renal function and no rhabdomyolysis. The events of flu like symptoms, myalgia, and anuria together were recorded as an SAE of ‘influenza like illness’ starting on Day 5 and ending approximately 8 days later, on Day 13. This SAE was of moderate severity and considered by the Investigator to be unlikely related to the study drug. The subject did not receive the planned dose of 150 mg IMB-1018972 under fed conditions in the second treatment period. The subject returned on Day 15 for a follow-up with safety assessments conducted as planned. The subject received 37.5 mg tramadol twice daily on Days 6 and 7 and 1000 mg paracetamol twice daily on Days 7 and 8 because of the flu like symptoms. The subject also reported mild TEAEs of back pain from Day 1 to Day 2 (not related), medical device site pruritus on Day 2 (not related), erythema on Day 2 (unlikely related), and burning sensation from Day 2 to Day 5 (unlikely related).

The SAE of ‘influenza like illness’ that led to the withdrawal of Subject 131 from the study was considered by the Investigator to be unlikely related to the study drug due to its weak time-relationship with study drug administration. The Investigator considers this SAE may have been caused by an infection.

### Concomitant Treatment

### SAD Part (and Integrated FE Arm)

Eighteen subjects in the SAD part (with integrated FE arm) received or took concomitant medication. Fifteen female subjects used contraception during the study. In addition, 4 subjects received concomitant medication as follows:
One subject (Subject 103; 50 mg IMB-1018972 under fasted conditions) received triamcinolone once daily for 2 days because of contact dermatitis on the chest (preferred term: contact dermatitis).
One subject (Subject 116 150 mg IMB-1018972 under fasted conditions) received 500 mg paracetamol once because of dizziness.
One subject (Subject 129; 150 mg IMB-1018972 under fasted conditions [fasted-fed group]) received 1000 mg paracetamol once or twice per day twice because of headache, and once because of muscular cramps of the upper legs (preferred term: muscle spasms). The same subject also received 5 mg oxycodone 4 times a day for 12 days, 1000 mg paracetamol 4 times a day for 5 days, 80 mg macrogol 4 times a day for 12 days, and 200 mg celecoxib once daily for 5 days because of tonsillitis.
One subject (Subject 131; 150 mg IMB-1018972 under fasted conditions [fasted-fed group]) received 37.5 mg tramadol twice daily for 2 days and 1000 mg paracetamol twice daily for 2 days because of flu like disease (preferred term: influenza like illness).

These medications were not considered to have influenced the outcome of the study

### MAD Part

Seven subjects in the MAD part received or took concomitant medication. Six female subjects used contraception during the study. In addition, 1 subject (Subject 221; placebo q12 h under fed conditions) received concomitant 500 mg paracetamol once because of a sore throat (preferred term: oropharyngeal pain).

These medications were not considered to have influenced the outcome of the study.

### Safety Conclusions

Overall, single oral IMB-1018972 doses and multiple oral IMB-1018972 doses of an IR formulation, were generally well tolerated by healthy male and female subjects. There were no findings of clinical relevance with respect to clinical laboratory, vital signs, 12-lead ECG, continuous cardiac monitoring (telemetry), or physical examination. Of note, there were no findings of hemodynamic changes, nor changes in the QTc-interval, after administration of IMB-1018972 as the IR formulations.
During the SAD part, the most common AEs were 6 TEAEs of flushing (reported terms were ‘niacin flush’ and ‘flushing neck’), of which 5 TEAEs were of moderate severity and 1 TEAE was of mild severity. Four subjects reported flushing after a single dose of 400 mg IMB-1018972 under fasted conditions, and 2 subjects of the FE arm reported flushing after a single dose of 150 mg IMB-1018972 under fasted conditions. These TEAEs were all considered by the Investigator to be related to the study drug. No subjects dropped out due to flushing and flushing was not considered a safety issue. Dose escalation beyond 400 mg IMB-1018972 IR did not proceed as
planned based on the PK exposure levels of IMB-1028814 and trimetazidine exceeding the target exposure levels in the 400 mg group and the findings of flushing at that dose. The predefined target exposure level was approximately 3 to 4 ‘trimetazidine equivalents’, ie, the ratio of the combined exposure of the active metabolites of IMB-1018972 to the single oral doses of 35 mg MR trimetazidine as seen in published literature.
There were no deaths reported during the study. Most TEAEs were of mild severity and no severe TEAEs were reported during the study. Overall, 12 of a total of 181 TEAEs were of moderate severity.
Two subjects were withdrawn from the study: 1 subject due to a moderate SAE of influenza like illness (unlikely related) and 1 due to a moderate TEAE of ALT increased (possibly related).
Overall, there was no clear dose dependency of the number and incidence of TEAEs.
Dosing under fed conditions appeared to attenuate the number and incidence of TEAEs in the FE arm of the SAD part.

### Discussion and Overall Conclusions

This was a double-blind, randomized, placebo-controlled study, consisting of a SAD part with integrated FE arm, and a MAD part to assess the safety, tolerability, and PK of ascending single and multiple oral doses of IMB-1018972 (IR formulation in the SAD and MAD parts), and single oral doses of a MR formulation of trimetazidine. The study started with the SAD part

### Safety Discussion

Overall, single oral IMB-1018972 doses and multiple oral IMB-1018972 doses of an IR formulation were generally well tolerated by healthy male and female subjects. There were no findings of clinical relevance with respect to clinical laboratory, vital signs, 12-lead ECG, continuous cardiac monitoring (telemetry), or physical examination. Of note, there were no findings of hemodynamic changes, nor changes in the QTc-interval, after administration of IMB-1018972 as the IR formulations.

Nicotinic acid (niacin) is an immediate hydrolysis product of IMB-1018972 and constitutes approximately 30% of the molecular mass of IMB-1018972. In this study, TEAEs of flushing, of which the characteristics were consistent with the flushing seen with the administration of niacin, were reported. All events were transient and resolved without intervention. No subjects dropped out and no modification of the dose was needed due to the TEAEs of flushing.

In the SAD (IR) part of the study, the most common AEs were 6 TEAEs of flushing (reported terms were ‘niacin flush’ and ‘flushing neck’), of which 5 TEAEs were of moderate severity and 1 TEAE was of mild severity. Four subjects reported flushing after a single dose of 400 mg IMB-1018972 under fasted conditions, and 2 subjects of the FE arm reported flushing after a single dose of 150 mg IMB-1018972 under fasted conditions.

In the 14-day multiple dose part of the study, subjects received 150 or 50 mg IR IMB-1018972 q12 h in the fed state. Six subjects in the 150 mg q12 h group reported single instances of flushing that were mild in severity. No TEAEs of flushing were reported following administration of 50 mg IR IMB-1018972 q12 h.

One subject was withdrawn from the study. One subject of the FE arm Group A4 was withdrawn from the study due to a moderate SAE of ‘influenza like illness’ following administration of a single oral dose of 150 mg IMB-1018972 under fasted conditions. The SAE of influenza like illness was considered by the Investigator unlikely to be related to the study drug.

The most frequently reported TEAEs during the study were of the SOC vascular disorders (mainly TEAEs of flushing), general disorders and administration site conditions, nervous system disorders, gastrointestinal disorders, and musculoskeletal and connective tissue disorders. The majority of the TEAEs reported during the study were considered by the Investigator not to be related to the study drug.

### Pharmacokinetics

Based on the single-dose and multiple-dose PK results obtained for IMB-1028814 and trimetazidine in this study.

### Safety—Conclusion

Overall, single oral IMB-1018972 doses and multiple oral IMB-1018972 doses of an IR formulation were generally well tolerated by healthy male and female subjects. There were no findings of clinical relevance with respect to clinical laboratory, vital signs, 12-lead ECG, continuous cardiac monitoring (telemetry), or physical examination. Of note, there were no findings of hemodynamic changes, nor changes in the QTc-interval, after administration of IMB-1018972 either as the IR or MR formulations.
During the SAD part, the most common AEs were 6 TEAEs of flushing (reported terms were ‘niacin flush’ and ‘flushing neck’), of which 5 TEAEs were of moderate severity and 1 TEAE was of mild severity. Four subjects reported flushing after a single dose of 400 mg IMB-1018972 under fasted conditions, and 2 subjects of the FE arm reported flushing after a single dose of 150 mg IMB-1018972 under fasted conditions. These TEAEs were all considered by the Investigator to be related to the study drug. No subjects dropped out due to flushing and flushing was not considered a safety issue. Dose escalation beyond 400 mg IMB-1018972 IR did not proceed as planned based on the PK exposure levels of IMB-1028814 and trimetazidine exceeding the target exposure levels in the 400 mg group and the findings of flushing at that dose. The predefined target exposure level was approximately 3 to 4 ‘trimetazidine equivalents’, ie, the ratio of the combined exposure of the active metabolites of IMB-1018972 to the single oral doses of 35 mg MR trimetazidine as seen in published literature.
There were no deaths reported during the study. Most TEAEs were of mild severity and no severe TEAEs were reported during the study. Overall, 12 of a total of 181 TEAEs were of moderate severity.
One subject was withdrawn from the study: 1 subject due to a moderate SAE of influenza like illness (unlikely related) and 1 due to a moderate TEAE of ALT increased (possibly related).
Overall, there was no clear dose dependency of the number and incidence of TEAEs.
Dosing under fed conditions appeared to attenuate the number and incidence of TEAEs in the FE arm of the SAD part.

### Pharmacokinetics—Conclusions

IMB-1018972 could be measured in only few plasma samples taken during this study.
When combining the single and multiple IMB-1018972 dose results under fasted and fed conditions, including those of the MR formulations, the initial hydrolysis of IMB-1018972 to IMB-1028814 and subsequent systemic bioavailability of IMB-1028814 was relatively rapid with median tmax ranging between 0.5 hours and 5 hours postdose for IMB-1028814, and between 1.5 hours and 8 hours postdose for trimetazidine. Median tmax did not increase with increasing IMB-1018972 dose.
The predefined stopping criterion for IMB-1028814 plasma exposure of 417,733 and 652,849 ng·h/mL for males and females, respectively, was not reached by any of the subjects during the SAD part or MAD part.
Following single oral IMB-1018972 doses in the range of 50 to 400 mg under fasted conditions, systemic exposure to IMB-1028814 and trimetazidine was dose proportional for Cmax, AUC₀₋ₜ, and AUC_{0-inf}.
No evidence for an effect of food was observed on the IMB-1028814 exposure parameters AUC₀₋ₜ and AUC_{0-inf} following administration of a single dose of 150 mg IMB-1018972. However, Cₘₐₓ was approximately 36% lower following administration of a single dose of 150 mg IMB-1018972 under fed conditions relative to administration under fasted conditions.
No evidence for an effect of food was observed on the trimetazidine exposure parameters Cₘₐₓ, AUC₀₋ₜ, and AUC_{0-inf} following administration of a single dose of 150 mg IMB-1018972.
Within 48 hours following administration of a single oral dose of IMB-1018972 over the range of 50 mg to 400 mg, on average between 3.99% and 5.74% of the dose was excreted in urine as IMB-1028814, and on average between 23.11% and 32.55% of the dose was excreted in urine as trimetazidine.
Within 48 hours following administration of a single oral dose of 35 mg trimetazidine, on average 54.47% of the dose was excreted in urine as trimetazidine.
Following 14 days of twice daily dosing with 150 mg and 50 mg IMB-10818972 under fed conditions, no relevant accumulation was observed of IMB-1028814 (R_{ac} of 1.18 and 1.10 for 150 mg and 50 mg, respectively), and accumulation of trimetazidine was modest (R_{ac} of 1.63 and 1.89 for 150 mg and 50 mg, respectively).

### Overall

In view of the positive risk/benefit profile and the observed PK characteristics of the IMB-1018972 metabolites IMB-1028814 and trimetazidine in this single-dose and multiple-dose FIH study, further clinical development of IMB-1018972 is warranted.

### Example 2

### Brief Study Summary

A pharmacodynamic study to evaluate the impact of the compound of formula (X) on myocardial energetics and metabolism in obese patients with Type 2 diabetes.

### Objectives

The primary objective is to evaluate the impact of 200 mg of formula (X) on rest and stress myocardial energetics (PCr/ATP). Other objectives include: to evaluate the metabolic response, specifically the impact on PDH flux using hyperpolarized ¹³C-pyruvate MRS, as a measure of the compound's ability to promote glucose oxidation; to assess the effect on cardiac systolic and diastolic function, as measured by cardiac magnetic resonance (CMR) and transthoracic echocardiography (TTE); to measure the impact on myocardial steatosis.

### Design and Treatments

This was a randomized study consisting of a population of 20 patients with Type 2 diabetes and a BMI≥30 kg/m². The first five patients were treated for four weeks; the subsequent 15 patients (patients 6-20) were treated for eight weeks (with five additional patients prn). Ten patients had hyperpolarized ¹³C-pyruvate MRS.

### Preliminary Safety Data

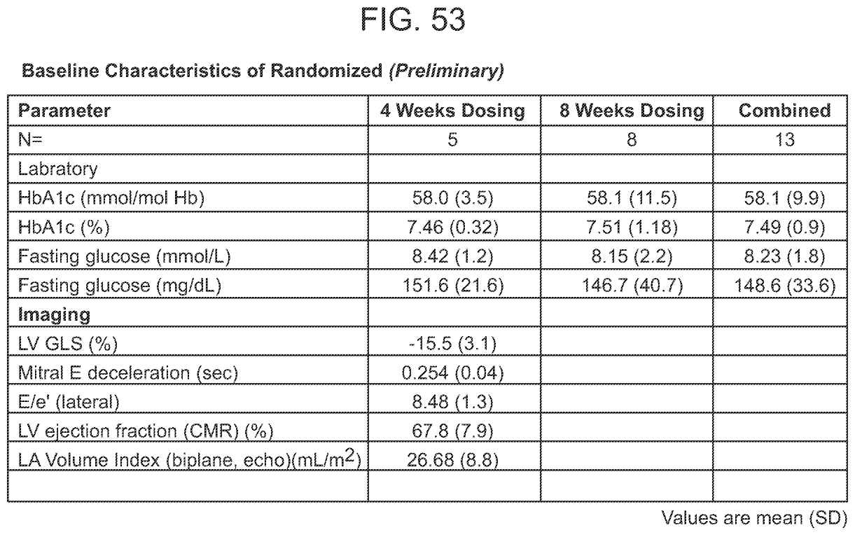

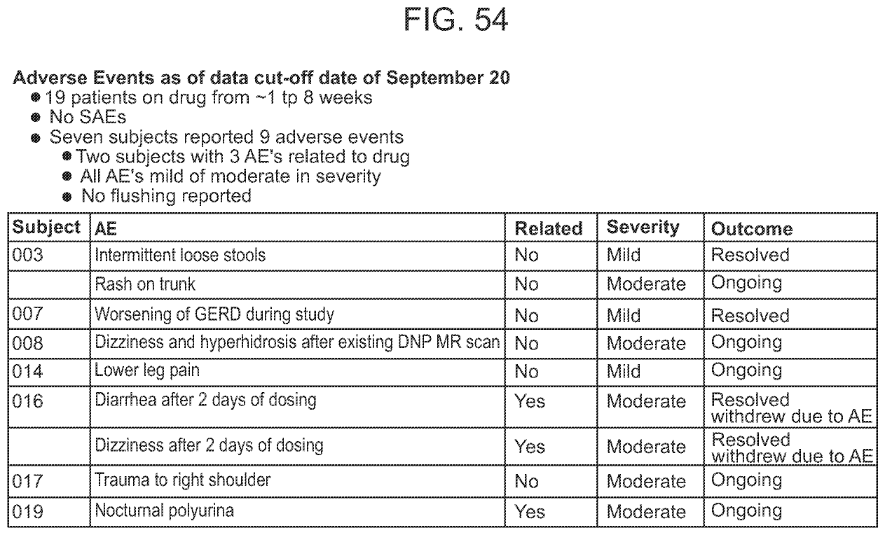

The preliminary baseline characteristics of randomized participants is detailed in FIG. 52. The preliminary baseline characteristics of completers is detailed in FIG. 53. FIG. 54 details adverse events as of the data cut-off date of September 20. Nineteen patients were on the drug from approximately one to eight weeks. There were no SAEs. Seven subjects reported nine adverse events. Two subjects had three drug-related AEs. All AEs were mild or moderate in severity. There was no flushing reported. Overall, the compound was well-tolerated.

### Preliminary Pharmacodynamic Data

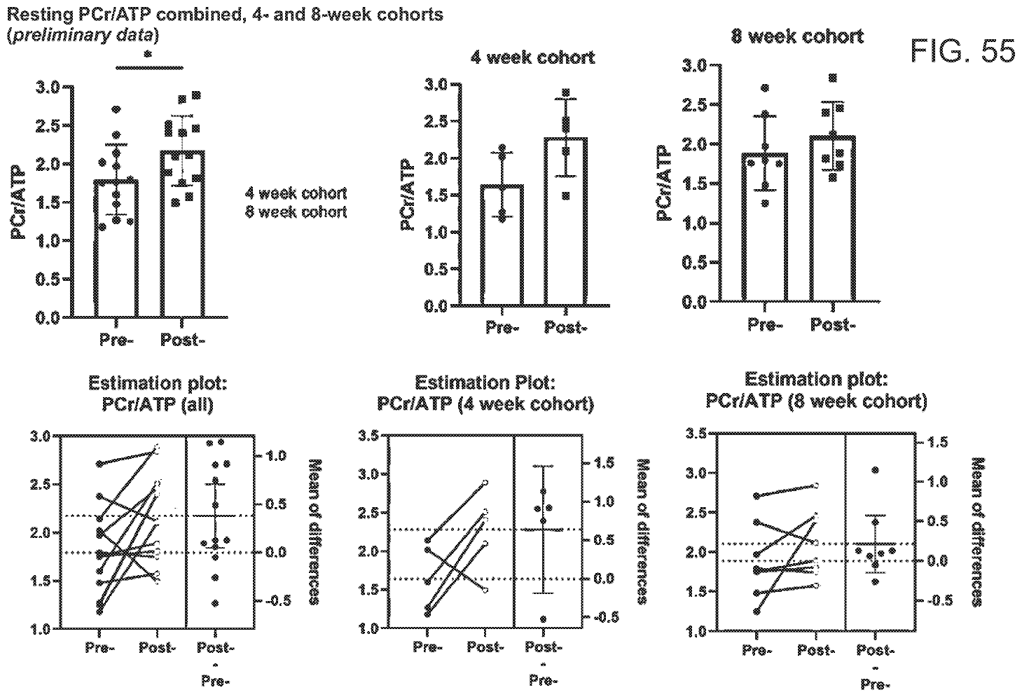

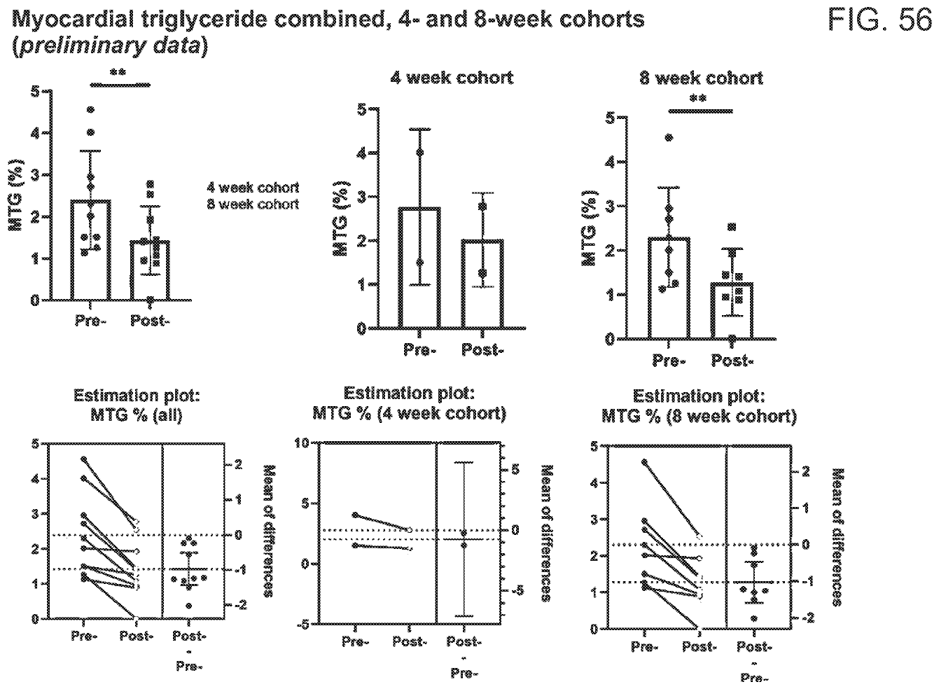

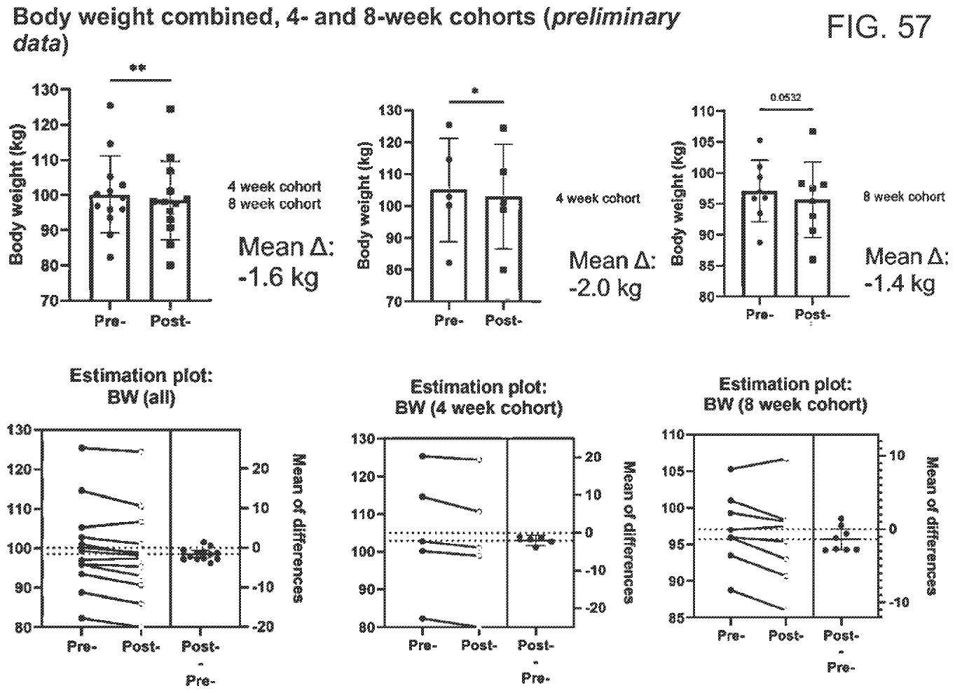

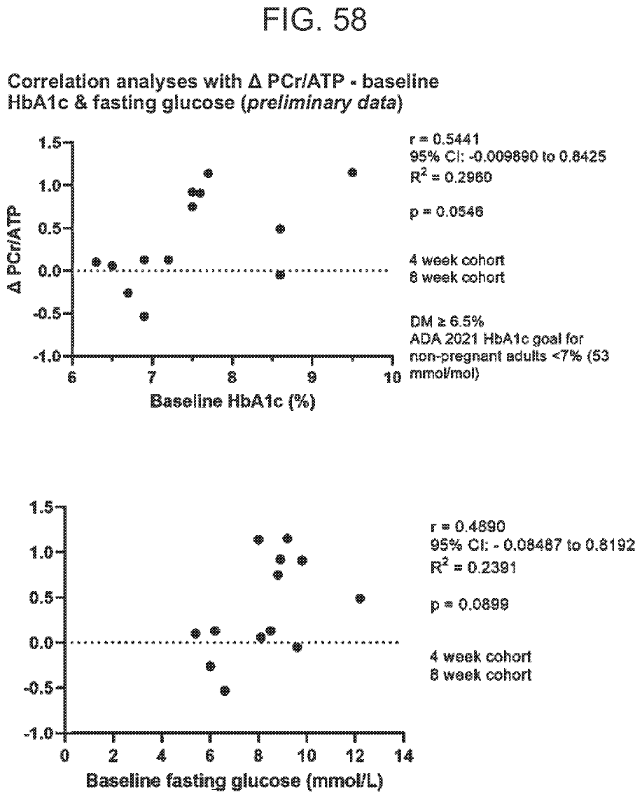

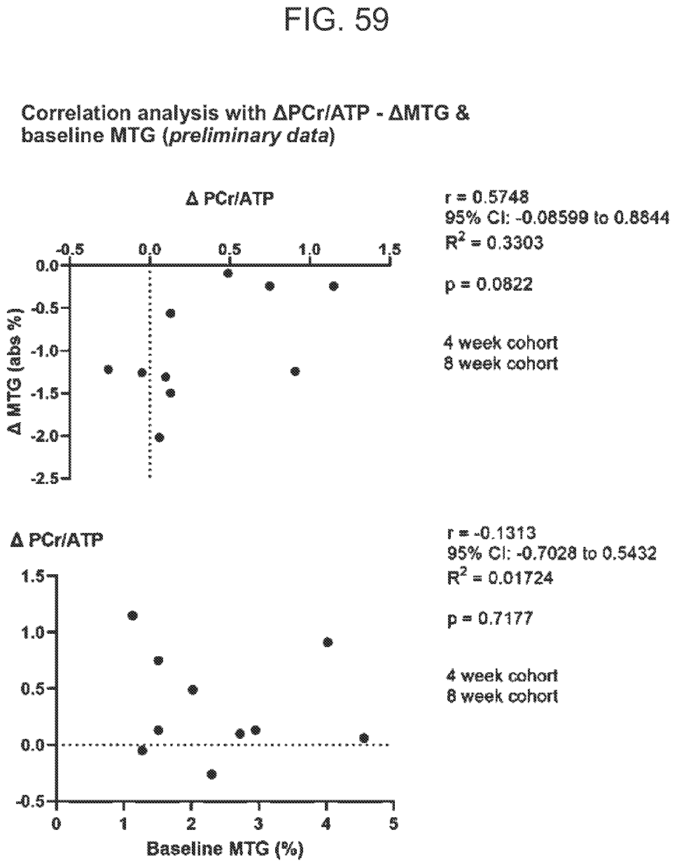

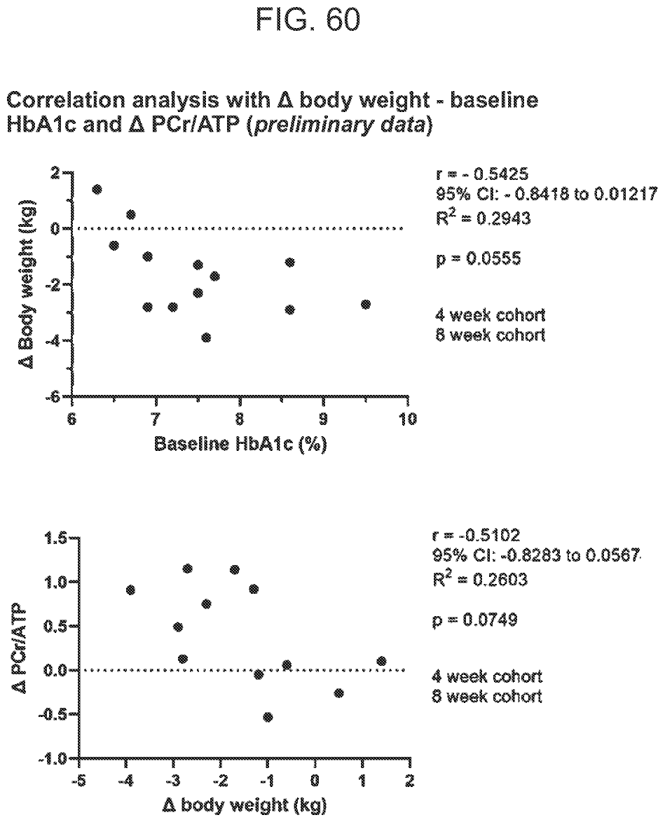

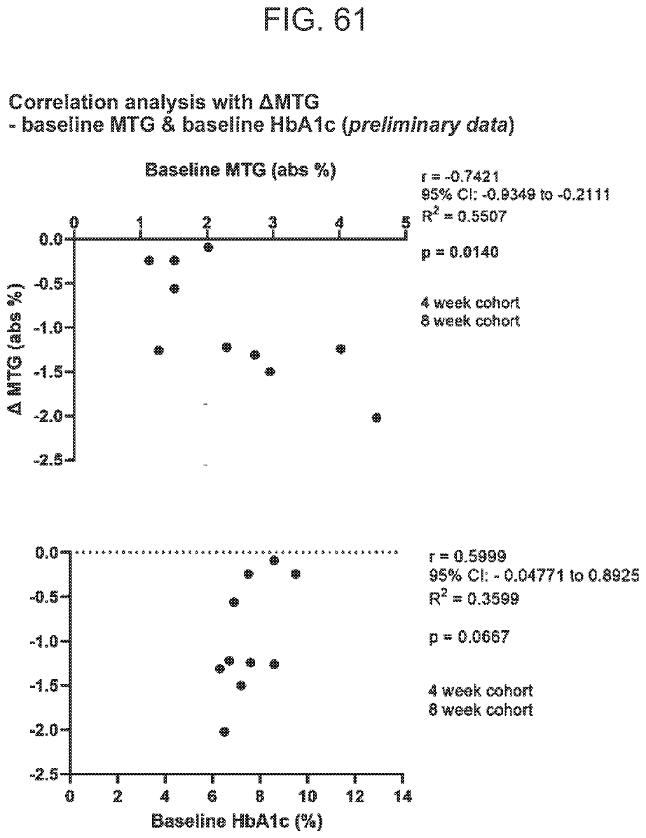

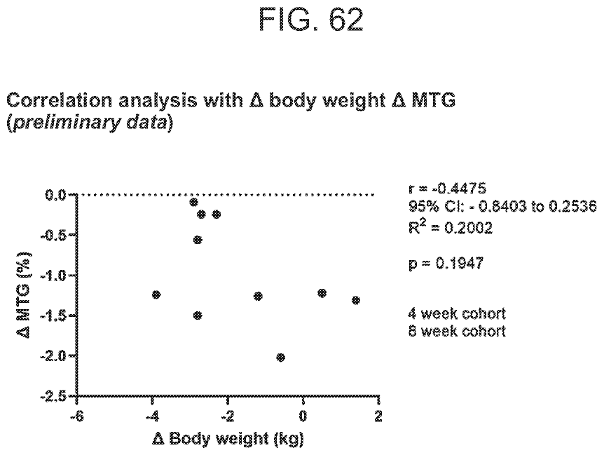

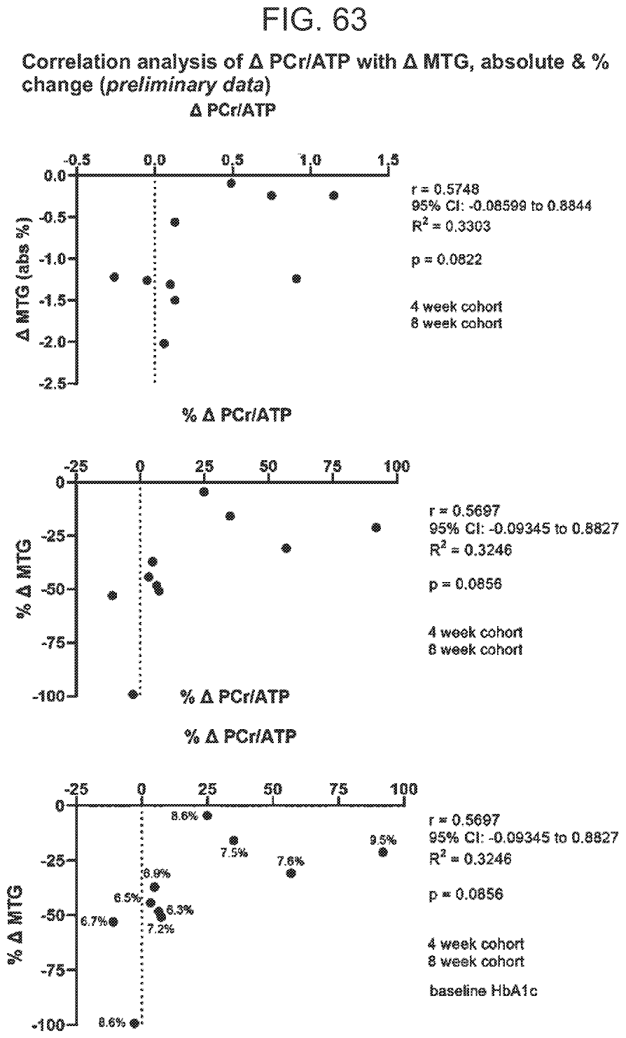

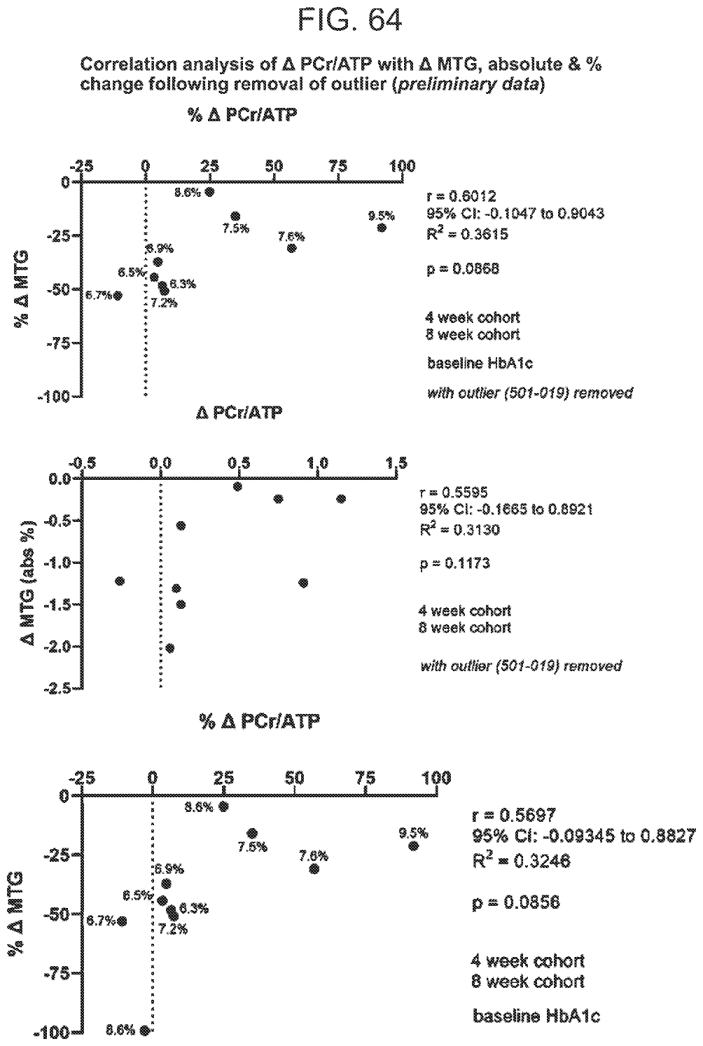

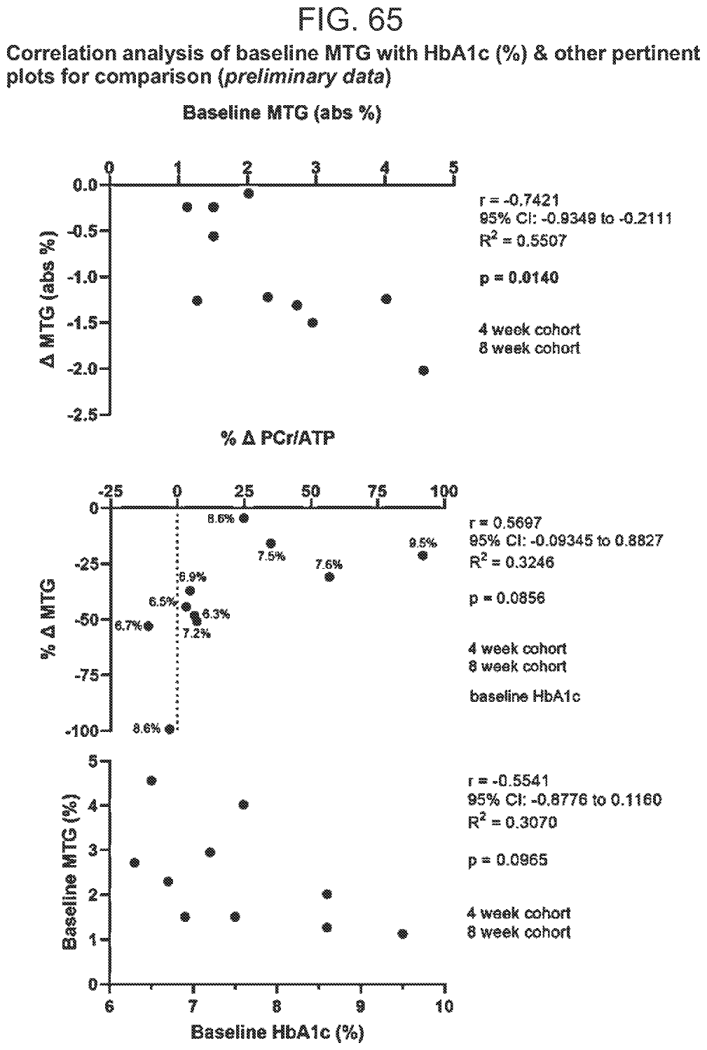

Analysis of interim data from the study confirms that compound of formula (X) is a robust metabolic modulator, with evidence to support target engagement and conceptualization as a cardiac mitotrope, i.e. an agent whose mechanism of action influences cardiac energetics with the potential to improve myocardial performance. Key results of the interim data include:
A significant and meaningful increase in cardiac energetics (PCr/ATP) in obese patients with Type 2 Diabetes is evident in the combined data from the 4- and 8-week cohorts.
   The energetic response appears to be greater for the 4-week (n=5) than the 8-week (n=8) cohort. This may be explained by differences between the cohorts.
   FIG. 55 details resting PCr/ATP combined for the 4- and 8-week cohorts.
PCr/ATP responses appear to be greater in patients with HbA1c>6.0%, 6.5% or 7%. Thus, HbA1c appears to be a predictor of cardiac energetic response. If confirmed, these data will guide patient selection in future studies of diabetic patients, e.g. in those with heart failure with preserved ejection fraction (HFpEF).
Those with more severe myocardial energetic impairment at baseline imaging experienced the greatest absolute PCr/ATP responses, i.e. the more severe the energetic deficit, the greater the mitotropic response.
A highly significant reduction in myocardial steatosis was demonstrated with a reduction in myocardial triglycerides in virtually all subjects. FIG. 56. Correlation analysis of change in myocardial triglycerides for baseline myocardial triglycerides and baseline HbA1c is detailed in FIG. 61.
Patients with the greatest reduction in myocardial triglyceride (TG) tended to have a smaller change in PCr/ATP. Correlation analysis of change in PCr/ATP with change in myocardial triglycerides (absolute and %) is detailed in FIG. 63, and following removal of a single outlier in FIG. 64.
Highlighting the clinical relevance of this, myocardial TG content is known to be independently associated with impaired LV diastolic function in Type 2 diabetes, a key contributor to impaired cardiac performance in HFpEF.
The impact of compound of formula (X) has the potential to reduce lipotoxicity to support better function of the diabetic myocardium and, by inference, other states associated with myocardial steatosis.
Short-term administration of formula (X) is associated with significant weight loss, consistent with rapid pharmacological effects extending beyond the heart. FIG. 57.
Weight loss was greater for those with higher baseline HbA1c (i.e. with greater baseline disturbance of glucose homeostasis). Correlation analysis with change in body weight and baseline HbA1C and change in PCr/ATP is detailed in FIG. 60. Correlation analysis of change in body weight with change in myocardial triglycerides is detailed in FIG. 62.
Contrary to expectations based on literature, higher baseline HbA1c was associated with lower myocardial triglycerides in this preliminary dataset.
Baseline HbA1c, and reduction in HbA1c with formula (X), were positively associated with increase in myocardial PCr/ATP, with a cutoff of baseline HbA1c of greater than 6.0%, 6.5% or 7% a predictor of PCr/ATP (i.e. energetic) response. FIG. 58.
Change in myocardial triglycerides was not significant with only weak inverse correlation p=0.7177, r=0.13. FIG. 59.
Correlation analysis for baseline myocardial triglycerides with HbA1 and other comparison plots are detailed in FIG. 65.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification, and guidance that can be adapted to the practice of this invention in its various embodiments and equivalents thereof.

1. A method of treating cardiac steatosis or a disorder associated with cardiac steatosis in a subject, the method comprising providing to a subject having, or at risk of developing, cardiac steatosis or a disorder associated with cardiac steatosis a composition comprising a compound of formula (X): 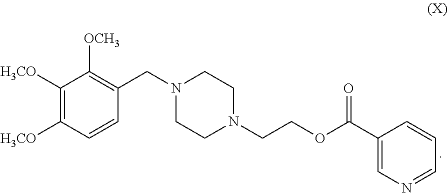 [SMILES: COc1ccc(CN2CCN(CCOC(=O)c3cccnc3)CC2)c(OC)c1OC]
or a pharmaceutically acceptable salt thereof.

2. The method of claim 1, wherein the composition is provided orally.

3. The method of claim 1, wherein the composition is provided in at least one dose per day.

4. The method of claim 1, wherein the composition is provided in multiple doses per day at a suitable interval.

5. The method of claim 3, wherein the at least one dose is provided daily for at least two weeks.

6. The method of claim 3, wherein the at least one dose comprises from about 25 mg to about 1000 mg of the compound of formula (X).

7. The method of claim 6, wherein the at least one dose comprises from about 50 mg to about 600 mg of the compound of formula (X).

8. The method of claim 7, wherein the at least one dose comprises about 100 mg to about 400 mg of the compound of formula (X).

9. The method of claim 8, wherein the at least one dose comprises about 200 mg of the compound of formula (X).

10. The method of claim 1, wherein the composition comprises a modified-release formulation.

11. A method of reducing myocardial triglycerides in a subject, the method comprising providing to a subject having, or at risk of developing, myocardial disease a composition comprising a compound of formula (X): 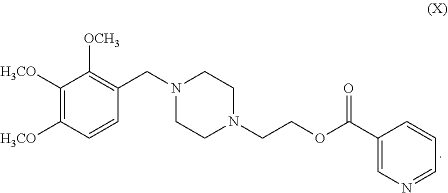 [SMILES: COc1ccc(CN2CCN(CCOC(=O)c3cccnc3)CC2)c(OC)c1OC]
or a pharmaceutically acceptable salt thereof.

12.-21. (canceled)

22. A method of reducing lipotoxicity in a subject, the method comprising providing to a subject having, or at risk of developing, lipotoxicity a composition comprising a compound of formula (X): 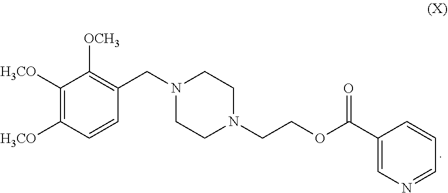 [SMILES: COc1ccc(CN2CCN(CCOC(=O)c3cccnc3)CC2)c(OC)c1OC]
or a pharmaceutically acceptable salt thereof.

23.-31. (canceled)

32. A method of inducing weight loss in a subject, the method comprising providing to a subject a composition comprising a compound of formula (X): 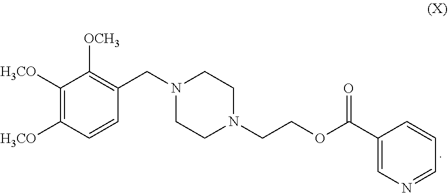 [SMILES: COc1ccc(CN2CCN(CCOC(=O)c3cccnc3)CC2)c(OC)c1OC]
or a pharmaceutically acceptable salt thereof.

33.-41. (canceled)

42. A method of treating cardiac dysfunction in a subject, the method comprising providing to a subject having an elevated level of HbA1c at least one dose per day of a composition comprising a compound of formula (X): 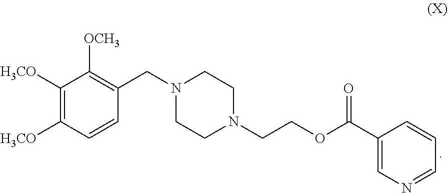 [SMILES: COc1ccc(CN2CCN(CCOC(=O)c3cccnc3)CC2)c(OC)c1OC]
or a pharmaceutically acceptable salt thereof.

43.-53. (canceled)

54. A method of treating diabetic cardiomyopathy in a subject, the method comprising providing to a subject having, or at risk of developing, diabetic cardiomyopathy or a disorder associated with diabetic cardiomyopathy, a composition comprising a compound of formula (X):

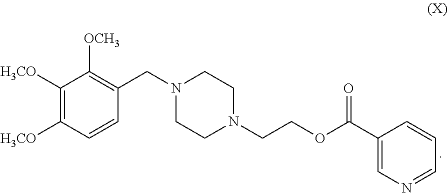 [SMILES: COc1ccc(CN2CCN(CCOC(=O)c3cccnc3)CC2)c(OC)c1OC]

55.-63. (canceled)
